(19) United States
(12) Patent Application Publication
(10) Pub. No.: US 2024/0101510 A1
(43) Pub. Date: Mar. 28, 2024

(54) APYRASE INHIBITORS

(71) Applicant: Texas Crop Science, Austin, TX (US)

(72) Inventors: Matthew Duncton, San Bruno, CA (US); Jeffrey D. Fowler, Wilkesboro, NC (US); John Clough, Marlow (GB); Tanya Sabharwal, Austin, TX (US); Simon Hiebert, Austin, TX (US)

(21) Appl. No.: 18/241,104

(22) Filed: Aug. 31, 2023

Related U.S. Application Data
(60) Provisional application No. 63/402,917, filed on Aug. 31, 2022.

(51) Int. Cl. C07C 311/21; C07C 247/10; C07D 213/42; C07D 215/12; C07D 217/12; C07D 237/08; C07D 239/26; C07D 241/12; C07D 317/58; C07D 319/16; C07D 333/34

(52) U.S. Cl. CPC C07C 311/21; C07C 247/10; C07D 213/42; C07D 215/12; C07D 217/12; C07D 237/08; C07D 239/26; C07D 241/12; C07D 317/58; C07D 319/16; C07D 333/34

(57) ABSTRACT

Disclosed herein are apyrase inhibitors of Formula (I)

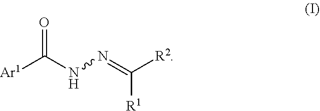

Also disclosed herein are methods for using the disclosed inhibitors, including in methods for protecting crops from pests. In one aspect the apyrase inhibitors are useful for enhancing the activity of pesticides for the protection of crops from pathogens and to support crop yield.

### CROSS-REFERENCE TO RELATED APPLICATIONS

This application claims the benefit of the earlier filing date of U.S. Provisional Application No. 63/402,917, filed Aug. 31, 2022, the disclosure of which is incorporated herein by reference.

### FIELD

The present disclosure relates to inhibitors of apyrase and methods for their use, in particular in the treatment of crops susceptible to pathogens.

### BACKGROUND

Crops are plagued worldwide by a variety of pathogens. Pathogens, such as insects, mites, nematodes, bacteria, weeds and fungi have developed an array of mechanisms for surviving pesticides, such as by sequestering, exporting or detoxifying them. The present inventors have discovered molecules and methods for potentiating the efficacy of pesticides by blocking certain mechanisms of resistance.

### SUMMARY

Disclosed herein are molecules and methods for their use in supporting crop viability and yield, by, for example, protecting crops from pests. In one embodiment, disclosed herein is a method for inhibiting apyrase enzymes, comprising contacting the apyrase with a compound of the formula

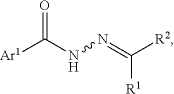

wherein Ar¹ is selected from aryl and heteroaryl;
R¹ is selected from hydrogen, C₃₋₆ cycloalkyl, C₁₋₆ alkyl, aralkyl, and C₁₋₃ haloalkyl;
R² is selected from alkyl, aryl and heteroaryl.

In further embodiments, an apyrase inhibitor as described herein is used in combination with one or more pesticide to treat a crop at risk.

The foregoing and other objects, features, and advantages of the invention will become more apparent from the following detailed description.

### DETAILED DESCRIPTION

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

“Administering” refers to any suitable mode of administration, to control a fungal pathogen, including, treatment of an extant crop, seeds, soil or combination thereof.

“Control” with reference to a fungal pathogen, means block, inhibit and/or eradicate a fungal pathogen and/or prevent the fungal pathogen from damaging a crop. In one embodiment, control refers to the reduction of one or more fungi to undetectable levels, or to the reduction or suppression of a fungus to acceptable levels as determined by one of ordinary skill in the art (for example, a crop grower). Determinations of acceptable levels of fungus reduction are based on a number of factors, including to the crop, pathogen, severity of the pathogen, use restrictions, economic thresholds and other factors known to those of ordinary skill in the art.

As used herein, the terms “enhancer” and “potentiator”, refer to a compound or compounds disclosed herein that enhance the effects of a pesticide. Without limitation to theory the present enhancer compounds disclosed herein may function by blocking one or more pathways by which a pathogen, such as a fungal pathogen evades toxicity, such as by detoxifying, sequestering or transporting a pesticide. In certain embodiment, the present compounds inhibit enzymatic apyrase activity which leads to the enhancement, accentuation or potentiation of a pesticide, such as an acaricide, antimicrobial, fungicide, herbicide, insecticide, molluscicide and/or nematocide. For example, when the enhancer or potentiator is used in conjunction with a fungicide, the combination of the potentiator and the fungicide enhances the fungicidal effect of the fungicide and/or renders a fungus that has become resistant to the fungicide susceptible to the fungicide as a result of the activity of the potentiator. Most often, these enhancers or potentiators do not themselves inhibit the fungus itself, nor do they have a detrimental effect on a living organism that is (or could be) infected with a fungus.

As used herein, the term “inoculation” refers to a method used to administer or apply an effective amount of a disclosed compound or formulation thereof to a target area of a field and/or plant. The inoculation method can be, but is not limited to, aerosol spray, pressure spray, direct watering, and dipping. Target areas of a plant could include, but are not limited to, the leaves, roots, stems, buds, flowers, fruit, seed of the plant, and bulbs of the plant including bulb, corm, rhizoma, stem tuber, root tuber and rhizophore. Inoculation can include a method wherein a plant is treated in one area (for example, the root zone or foliage) and another area of the plant becomes protected (for example, foliage is inoculated when a disclosed compound is applied in the root zone or new growth when applied to foliage).

As used herein, the terms “wettable granule”, “water dispersible granule”, and “dispersible granule” refer to a solid granular formulation prepared by a granulation process, optionally containing fine particles of polymer-associated active ingredient, or aggregates of the same, a wetting agent and/or a dispersant, and optionally an inert filler. Wettable granules can be stored as a formulation, and can be provided to the market and/or end user without further processing. In some embodiments, they can be placed in a water-soluble bag for ease of use by the end user. In practical application, wettable granules are prepared for application by the end user. The wettable granules are mixed with water in the end user's spray tank to the proper dilution for the particular application. Dilution can vary by crop, fungal pathogen, time of year, geography, local regulations, and intensity of infection among other factors. Once properly diluted, the solution can be applied by spraying.

As used herein, the terms “wettable powder”, “water dispersible powder”, and “dispersible powder”, refer to a solid powdered formulation that contains active ingredient, optionally associated with a polymer, or aggregates of the same, and optionally one or more of a dispersant, a wetting agent, and an inert filler. Wettable powders can be stored as a formulation, and can be provided to the market and/or end user without further processing. In some embodiments, they can be placed in a water-soluble bag for ease of use by the end user. In practical application, a wettable powder is prepared for application by the end user. The wettable powder is mixed with water in the end user's spray tank to the proper dilution for the particular application. Dilution can vary by crop, target pathogen, time of year, geography, local regulations, and intensity infection or pathogen load, among other factors. Once properly diluted, the solution can be applied by spraying.

As used herein, the term “high solids liquid suspension” refers to a liquid formulation that contains fine particles of active ingredient or fine polymer particles associated with active ingredient, or aggregates of the same, a wetting agent and/or a dispersant, an anti-freezing agent, optionally an anti-settling agent or thickener, optionally a preservative, and water or oil as a carrier. High solids liquid suspensions can be stored as a formulation, and can be provided to the market and/or end user without further processing. In practical application, high solids liquid suspensions are prepared for application by the end user. The high solids liquid suspensions are mixed with water or oil in the end user's spray tank to the proper dilution for the particular application. Dilution can vary by crop, target pathogen, time of year, geography, local regulations, and intensity of infection or pathogen load among other factors. Once properly diluted, the solution or suspension can be applied by spraying.

As used herein, the term “phytologically acceptable” refers to compositions, diluents, excipients, and/or carriers that are generally applicable for use with any part of a plant during any part of its life cycle, including but not limited to seeds, seedlings, plant cells, plants, or flowers. The compositions can be prepared according to procedures, methods and formulas that are known to those of skill in the agricultural arts. Following the teachings of the present disclosure the artist skilled in the agricultural and/or chemical arts can readily prepare a desired composition. Most commonly, the compounds of the present invention can be formulated to be stored, and/or applied, as aqueous or non-aqueous suspensions or emulsions prepared neat or from concentrated formulations of the compositions. Alternatively the compounds of the present invention can be formulated for use in aerosol-generating equipment for application to agricultural produce stored in sealed chambers—an application method known as fogging. Water-soluble, water-suspendable or emulsifiable formulations comprising the presently disclosed compounds can also be converted into or formulated as solids (for example, wettable powders), which can then be diluted into a final formulation. In certain formulations, the compositions of the present disclosure can also be provided in growth media, such as in vitro media for growth of plant or other types of cells, in laboratory plant growth media, in soil, or for spraying on seeds, seedlings, roots, stems, stalks, leaves, flowers or the entire plant.

Compounds herein can include all stereoisomers, including E and Z isomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, haloalkyl groups, alkenyl groups, haloalkenyl groups, alkynyl groups, haloalkynyl groups, alkoxy groups, aryl groups, aryloxy groups, aralkyl groups, arylalkoxy groups, heterocyclylalkyl groups, heteroaryl groups, cycloalkyl groups, acyl groups, acyloxy groups, carbamate groups, amide groups, ureido groups, epoxy groups, and ester groups.

“Alkyl” refers to an optionally substituted straight-chain, or optionally substituted branched-chain saturated hydrocarbon. Non-limiting examples of alkyl groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. In some cases alkyl refers to a group having from one to about ten carbon atoms, or from one to six carbon atoms, wherein an sp³-hybridized carbon of the alkyl residue is attached to the rest of the molecule by a single bond. Whenever it appears herein, a numerical range such as “C₁₋₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀ alkyl, a C₁₋₉ alkyl, a C₁₋₈ alkyl, a C₁₋₇ alkyl, a C₁₋₆ alkyl, a C₁₋₅ alkyl, a C₁₋₄ alkyl, a C₁₋₃ alkyl, a C₁₋₂ alkyl, or a C₁ alkyl.

Examples of alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen. Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

“Alkenyl” refers to an optionally substituted straight-chain, or optionally substituted branched-chain hydrocarbon having one or more carbon-carbon double-bonds. The olefin or olefins of an alkenyl group can be, for example, E, Z, cis, trans, terminal, or exo-methylene. An alkenyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybutan-1-yl, 7-hydroxy-7-methyloct-4-en-2-yl, and 7-hydroxy-7-methyloct-3,5-dien-2-yl.

Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon. The triple bond of an alkynyl group can be internal or terminal. An alkynyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

A haloalkyl group can be any alkyl group substituted with any number of halogen atoms, for example, fluorine, chlorine, bromine, and iodine atoms. A halo-alkenyl group can be any alkenyl group substituted with any number of halogen atoms. A haloalkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

The term “acyl” refers to the groups HC(O)—, alkyl-C(O)—, cycloalkyl-C(O)—, cycloalkenyl-C(O)—, aryl-C(O)—, heteroaryl-C(O)— and heterocyclyl-C(O)— where alkyl, cycloalkyl, cycloalkenyl, aryl, heteroaryl, and heterocyclyl are as described herein. By way of example acyl groups include acetyl and benzoyl groups.

“Alkoxy” refers to a radical of the formula —OR^{a} where R^{a} is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocyclylalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-Cis cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₈ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Non-limiting examples of cycloalkyl groups include cyclopropyl, cyclobutyl, cyclopentyl, cyclohexyl, cycloheptyl, and cyclooctyl groups. Cycloalkyl groups also include fused-, bridged-, and spiro-bicycles and higher fused-, bridged-, and spiro-systems. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Monocyclic cycloalkyls include, for example, cyclopropyl, cyclobutyl, cyclopentyl, cyclohexyl, cycloheptyl, and cyclooctyl.

Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl.

Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁₋₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocyclyl” refers to a stable 3- to 24-membered heterocycle. A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, pyrimidine, pyrazine, pyridazine, piperidine, succinimide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

“Heterocyclylalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocyclylalkyl radical may be a monocyclic, bicyclic, tricyclic, or tetracyclic ring system, which may include fused (when fused with an aryl or a heteroaryl ring, the heterocyclylalkyl is bonded through a non-aromatic ring atom) or bridged ring systems; and the nitrogen, carbon, or sulfur atoms in the heterocyclylalkyl radical may be optionally oxidized; the nitrogen atom may be optionally quaternized.

Representative heterocyclylalkyls include, but are not limited to, heterocyclylalkyls having from two to fifteen carbon atoms (C₂-Cis heterocyclylalkyl), from two to ten carbon atoms (C₂-C₁₀ heterocyclylalkyl), from two to eight carbon atoms (C₂-C₈ heterocyclylalkyl), from two to six carbon atoms (C₂-C₆ heterocyclylalkyl), from two to five carbon atoms (C₂-C₅ heterocyclylalkyl), or two to four carbon atoms (C₂-C₄ heterocyclylalkyl). In some embodiments, the heterocyclylalkyl is a 3- to 6-membered heterocyclylalkyl. In some embodiments, the cycloalkyl is a 5- to 6-membered heterocyclylalkyl. Examples of such heterocyclylalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocyclylalkyl also includes all ring forms of the carbohydrates, including but not limited to, the monosaccharides, the disaccharides, and the oligosaccharides. It is understood that when referring to the number of carbon atoms in a heterocyclylalkyl, the number of carbon atoms in the heterocyclylalkyl is not the same as the total number of atoms (including the heteroatoms) that make up the heterocyclylalkyl (i.e. skeletal atoms of the heterocyclylalkyl ring). Unless stated otherwise specifically in the specification, a heterocyclylalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heterocyclylalkyl is optionally substituted with oxo, halogen, methyl, ethyl, —CN, —CF₃, —OH, —OMe, —NH₂, or —NO₂. In some embodiments, a heterocyclylalkyl is optionally substituted with oxo, halogen, methyl, ethyl, —CN, —CF₃, —OH, or —OMe. In some embodiments, the heterocyclylalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocyclylalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

### II. Compounds

In one embodiment enhancers of pesticidal activity disclosed herein include those having Formula (I)

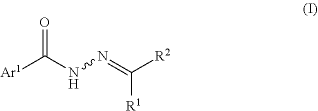

wherein Ar¹ is selected from aryl and heteroaryl;

R¹ is selected from hydrogen, C₃₋₆ cycloalkyl, C₁₋₆ alkyl, aralkyl, and C₁₋₃ haloalkyl;

R² is selected from alkyl, aryl and heteroaryl; provided that the compound does not have the formula

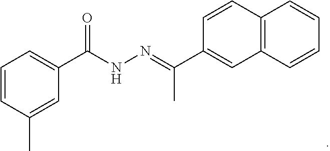 [SMILES: C/C(=N\NC(=O)c1cccc(C)c1)c1ccc2ccccc2c1]

With reference to Formula (I), the bond “ [symbol] ” indicates that the carbon—nitrogen double bond may be cis or trans and the compound may be the E or Z isomer. Thus, in one embodiment of compounds according to Formula (I), provided are compounds of Formula (Ia)

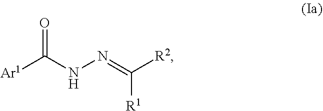

wherein Ar¹ is selected from aryl and heteroaryl;

R¹ is selected from hydrogen, C₃₋₆ cycloalkyl, C₁₋₆ alkyl, aralkyl, and C₁₋₃ haloalkyl;

R² is selected from alkyl, aryl and heteroaryl; provided that the compound does not have the formula

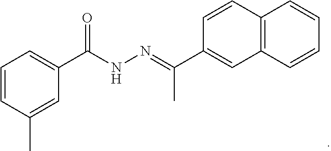 [SMILES: C/C(=N\NC(=O)c1cccc(C)c1)c1ccc2ccccc2c1]

In another embodiment, compounds of Formula (I) have Formula (Ib)

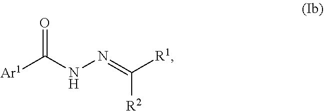

wherein Ar¹ is selected from aryl and heteroaryl;

R¹ is selected from hydrogen, C₃₋₆ cycloalkyl, C₁₋₆ alkyl, aralkyl, and C₁₋₃ haloalkyl;

R² is selected from alkyl, aryl and heteroaryl.

In certain embodiments, compounds of Formulas (I), (Ia) and (Ib) have Formula

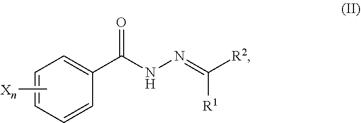

wherein X is, for each occurrence, independently selected from R^{a}, R^{b}, R^{a} substituted with one or more of the same or different R^{b}, —OR^{a} substituted with one or more of the same or different R^{b} or R^{d}, or
—(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —NH[(CH₂)ₘR^{b}], —N[(CH₂)ₘR]₂, —NH—C(O)—NH—(CH₂)ₘ—R^{b}, —NH—C(O)—(CH₂)ₘ—C HR^{b}R^{b};
or two X substituents together with the atoms to which they are attached form a 5 to 8-membered aryl, cycloalkyl, heterocyclylalkyl or heteroaryl which may optionally include one or more of the same or different additional heteroatoms and which may optionally be substituted with one or more of the same or different R^{b} groups;
each R^{a} is independently selected from the group consisting of C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl, 3-8 membered heterocyclylalkyl, 4-11 membered heterocyclylalkyl alkyl, 5-10 membered heteroaryl and 6-16 membered heteroarylalkyl;

R^{b} is a group independently selected from the group consisting of ═O, —OR^{d}, C₁₋₃ haloalkyloxy, —OCF₂H, —OCH₂F, —OCF₃, ═S, —SR^{d}, —SCF₃, —SF₅, ═NR^{d}, ═NOR^{d}, —NR^{c}R^{c}, halogen, —CF₃, —CN, —NO₂, —S(O)R^{d}, —S(O)₂R^{d}, —S(O)₂CF₃, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c} is independently R^{a}, or, alternatively, two R^{c} are taken together with the nitrogen atom to which they are bonded to form a 5 to 8-membered heterocyclylalkyl or heteroaryl which may optionally include one or more of the same or different additional heteroatoms and which may optionally be substituted with one or more of the same or different R^{b} groups;

each R^{d} is independently hydrogen or C₁₋₆ alkyl;

each m is independently an integer from 1 to 3; and
each n is independently an integer from 0 to 3.

In certain embodiments, compounds of Formulas (I), (Ia) and (Ib) have X selected from C₁₋₆ alkyl, —OR^{a}, —S(O)₂NR^{c}R^{c} and halogen.

In particular embodiments disclosed herein, including compounds of Formulas (I), (Ia), (Ib) and (II), Ar¹ is optionally substituted aryl, such as optionally substituted phenyl. In certain examples of such compounds X is —OR^{a} substituted with one or more of the same or different R^{b} or R^{d}. In one such embodiment wherein Ar¹ is phenyl, n is two and X is —OR^{a} wherein each R^{a} is selected from the group consisting of C₁₋₆ alkyl. In a particular embodiment, inhibitor compounds have Formula (IIa)

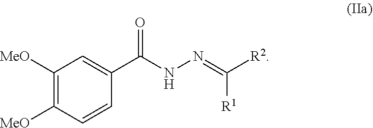

In particular embodiments of Formulas (I), (Ia), (Ib), (II) and (IIa), R¹ is hydrogen and R² is selected from hydrogen alkyl, aryl and heteroaryl. In one embodiment of the formulas above R¹ is hydrogen and R² is selected from hydrogen and methyl, such as wherein R¹ and R² are hydrogen.

In certain embodiments described herein, including embodiments of Formulas (I), (Ia) and (Ib), Ar¹ is heteroaryl, such as wherein Ar¹ is monocyclic heteroaryl or bicyclic heteroaryl.

In particular embodiments of Formulas (I), (Ia) and (Ib), Ar¹ is monocyclic heteroaryl, such as wherein Ar¹ is optionally substituted pyridyl. In one embodiment, compounds of Formulas (I), (Ia) and (Ib), have Formula (III)

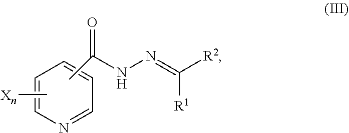

wherein X is, for each occurrence, independently selected from R^{a}, R^{b}, R^{a} substituted with one or more of the same or different R^{b}, —OR^{a} substituted with one or more of the same or different R^{b} or R^{d}, or
—(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂,
—O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b},
—C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b},
—O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b},
—NH—(CHR^{a})ₘ—R^{b}, —NH[(CH₂)ₘR^{b}], —N[(CH₂)ₘR]₂, —NH—C(O)—NH—(CH₂)ₘ—R^{b}, —NH—C(O)—(CH₂)ₘ—C HR^{b}R^{b};
or two X substituents together with the atoms to which they are attached to form a 5 to 8-membered aryl, cycloalkyl, heterocyclylalkyl or heteroaryl which may optionally include one or more of the same or different additional heteroatoms and which may optionally be substituted with one or more of the same or different R^{b} groups;
each R^{a} is independently selected from the group consisting of C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl, 3-8 membered heterocyclylalkyl, 4-11 membered heterocyclylalkyl alkyl, 5-10 membered heteroaryl and 6-16 membered heteroarylalkyl;
R^{b} is a group independently selected from the group consisting of ═O, —OR^{d}, C₁₋₃ haloalkyloxy, —OCF₂H, —OCH₂F, —OCF₃, ═S, —SR^{d}, —SCF₃, —SF₅, ═NR^{d}, ═NOR^{d}, —NR^{c}R^{c}, halogen, —CF₃, —CN, —NO₂, —S(O)R^{d}, —S(O)₂R^{d}, —S(O)₂CF₃, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and [NR^{a}C(NR^{a})]ₙNR^{c}R^{c}; each R^{c} is independently R^{a}, or, alternatively, two R^{c} are taken together with the nitrogen atom to which they are bonded to form a 5 to 8-membered heterocyclylalkyl or heteroaryl which may optionally include one or more of the same or different additional heteroatoms and which may optionally be substituted with one or more of the same or different R^{b} groups;

each R^{d} is independently hydrogen or C₁₋₆ alkyl;

each m is independently an integer from 1 to 3; and
each n is independently an integer from 0 to 3.

In one embodiment, compounds of Formulas (I), (Ia), (Ib) and (III) have Formula (IIIa)

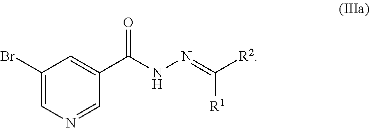

In another embodiment of Formulas (I), (Ia) and (Ib), wherein Ar¹ is monocyclic heteroaryl, compounds have Formula (IV)

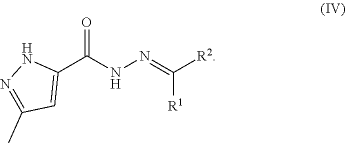

In one embodiment of Formulas (I), (Ia) and (Ib), wherein Ar¹ is bicyclic heteroaryl, compounds have Formula (V)

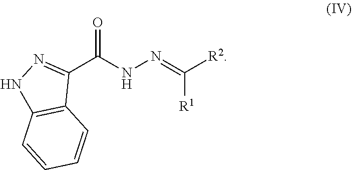

In another embodiment of Formulas (I), (Ia) and (Ib), wherein Ar¹ is bicyclic heteroaryl, the compound has the formula

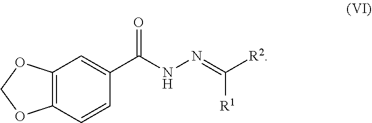

In particular embodiments of Formulas (I), (Ia), (Ib), (II), (IIa), (III), (IIIa), (IV), (V) and (VI), R² is alkyl, such as methyl. In other embodiments of Formulas (I), (Ia), (Ib), (II), (IIa), (III), (IIIa), (IV), (V) and (VI), R² is heteroaryl or aryl. In one such embodiment, R² is aryl and in a particular embodiment of such compounds R² is optionally substituted phenyl, such as in compounds of Formula (VII)

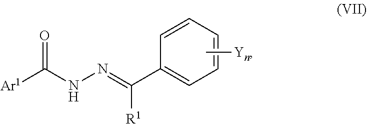

wherein Y is, for each occurrence, independently selected from R^{a}, R^{b}, R^{a} substituted with one or more of the same or different R^{b}, —OR^{a} substituted with one or more of the same or different R^{b} or R^{d}, or

—(CH₂)ₘ—R^{b}, —(CHR^{a})ₘ—R^{b}, —O—(CH₂)ₘ—R^{b}, —O—(CH₂)ₘ—R^{b}, —O—CHR^{a}R^{b}, —O—CR^{a}(R^{b})₂,
—O—(CHR^{a})ₘ—R^{b}, —O—(CH₂)ₘ—CH[(CH₂)ₘR^{b}]R^{b}, —O—(CHR^{a})ₘ—R^{b}, —C(O)NH—(CH₂)ₘ—R^{b},
—C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b},
—O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b},
—NH—(CHR^{a})ₘ—R^{b}, —NH[(CH₂)ₘR^{b}], —N[(CH₂)ₘR]₂, —NH—C(O)—NH—(CH₂)ₘ—R^{b}, —NH—C(O)—(CH₂)ₘ—C HR^{b}R^{b};
or two Y substituents together with the atoms to which they are attached to form a 5 to 8-membered aryl, cycloalkyl, heterocyclylalkyl or heteroaryl which may optionally include one or more of the same or different additional heteroatoms and which may optionally be substituted with one or more of the same or different include alkyl, cycloalkyl, and R^{b} groups;
each R^{a} is independently selected from the group consisting of C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl, 3-8 membered heterocyclylalkyl, 4-11 membered heterocyclylalkyl alkyl, 5-10 membered heteroaryl and 6-16 membered heteroarylalkyl;
R^{b} is a group independently selected from the group consisting of ═O, —OR^{d}, C₁₋₃ haloalkyloxy, —OCF₂H, —OCH₂F, —OCF₃, ═S, —SR^{d}, —SCF₃, —SF₅, ═NR^{d}, ═NOR^{d}, —NR^{c}R^{c}, halogen, —CF₃, —CN, —NO₂, —S(O)R^{d}, —S(O)₂R^{d}, —S(O)₂CF₃, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
   —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c}; each R^{c} is independently R^{a}, or, alternatively, two R^{c} are taken together with the nitrogen atom to which they are bonded to form a 5 to 8-membered heterocyclylalkyl or heteroaryl which may optionally include one or more of the same or different additional heteroatoms and which may optionally be substituted with one or more of the same or different R^{b} groups;
each R^{d} is independently hydrogen or C₁₋₆ alkyl;
   each m is independently an integer from 1 to 3; and
   each n is independently an integer from 0 to 3.

In still further embodiments of Formulas (I), (Ia), (Ib), (II), (IIa), (III), (IIIa), (IV), (V) and (VI), compounds disclosed herein include those of Formula (VIII)

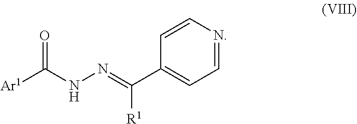

Specific examples of apyrase inhibitors according to the present disclosure and Formulas (I), (Ia), (Ib), (II), (IIa), (III), (IIIa), (IV), (V) (VI), (VII) and/or (VIII), for use to enhance the activity of an agricultural or horticultural pesticide as described herein are illustrated below in Table 1:

TABLE 1

| ID | Structure | Name |
|---|---|---|
| I-1 | 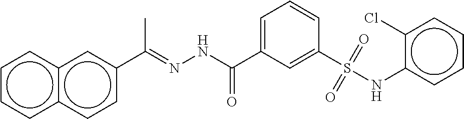 | (E)-N-(2-chlorophenyl)-3-(2-(1-(naphthalen- 2-yl)ethylidene)hydrazine-1- carbonyl)benzenesulfonamide |
| I-2 | 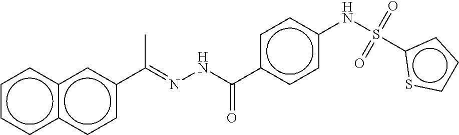 | (E)-N-(4-(2-(1-(naphthalen-2- yl)ethylidene)hydrazine-1- carbonyl)phenyl)thiophene-2-sulfonamide |
| I-3 | 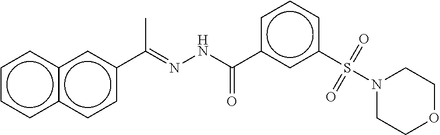 | (E)-3-(morpholinosulfonyl)-N′-(1- (naphthalen-2-yl)ethylidene)benzohydrazide |
| I-4 | 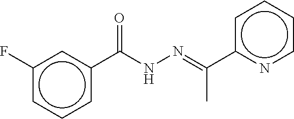 | (E)-3-fluoro-N′-(1-(pyridin-2- yl)ethylidene)benzohydrazide |
| I-5 | 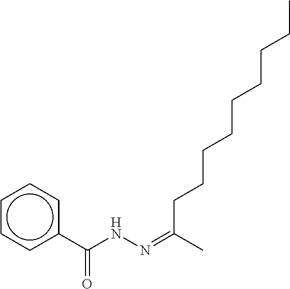 | (Z)-N′-(undecan-2-ylidene)benzohydrazide |
| I-6 | 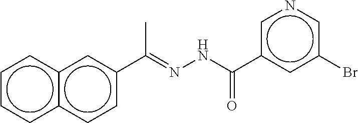 | (E)-5-bromo-N′-(1-(naphthalen-2- yl)ethylidene)nicotinohydrazide |
| I-7 | 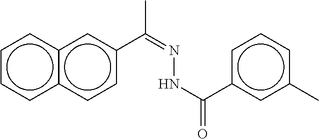 | (Z)-3-methyl-N′-(1-(naphthalen-2- yl)ethylidene)benzohydrazide |
| I-8 | 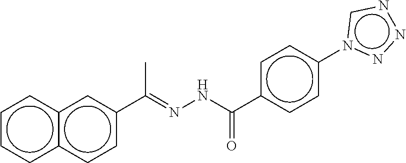 | (E)-N′-(1-(naphthalen-2-yl)ethylidene)-4-(1H- tetrazol-1-yl)benzohydrazide |
| I-9 | 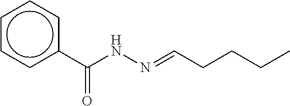 | (E)-N′-pentylidenebenzohydrazide |
| I-10 | 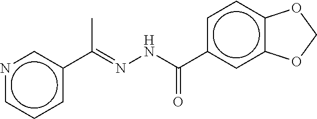 | (E)-N′-(1-(pyridin-3- yl)ethylidene)benzo[d][1,3]dioxole-5- carbohydrazide |
| I-11 | 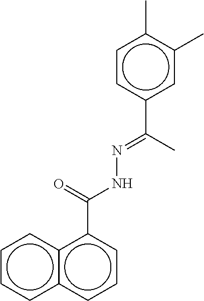 | (E)-N′-(1-(3,4-dimethylphenyl)ethylidene)-1- naphthohydrazide |
| I-12 | 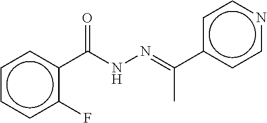 | (E)-2-fluoro-N′-(1-(pyridin-4- yl)ethylidene)benzohydrazide |
| I-13 | 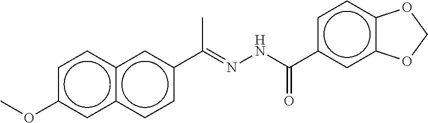 | (E)-N′-(1-(6-methoxynaphthalen-2- yl)ethylidene)benzo[d][1,3]dioxole-5- carbohydrazide |
| I-14 | 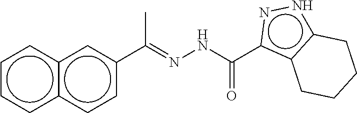 | (E)-N′-(1-(naphthalen-2-yl)ethylidene)- 4,5,6,7-tetrahydro-1H-indazole-3- carbohydrazide |
| I-15 | 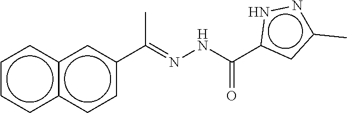 | (E)-3-methyl-N′-(1-(naphthalen-2- yl)ethylidene)-1H-pyrazole-5-carbohydrazide |
| I-16 | 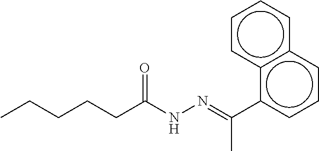 | (E)-N′-(1-(naphthalen-1- yl)ethylidene)hexanehydrazide |
| I-17 | 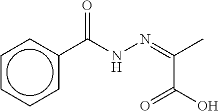 | (Z)-2-(2-benzoylhydrazineylidene)propanoic acid |
| I-18 | 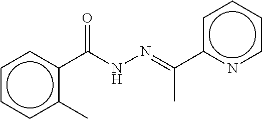 | (E)-2-methyl-N′-(1-(pyridin-2- yl)ethylidene)benzohydrazide |
| I-19 | 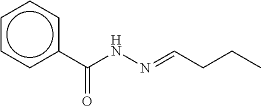 | (E)-N′-butylidenebenzohydrazide |
| I-20 | 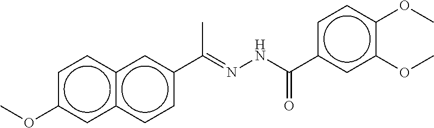 | (E)-3,4-dimethoxy-N′-(1-(6- methoxynaphthalen-2- yl)ethylidene)benzohydrazide |
| I-21 | 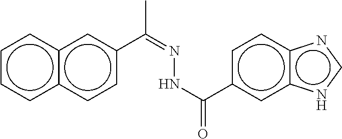 | (Z)-N′-(1-(naphthalen-2-yl)ethylidene)-1H- benzo[d]imidazole-6-carbohydrazide |
| I-22 | 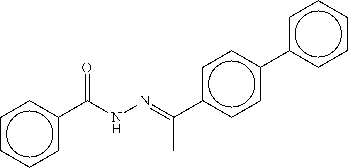 | (E)-N′-(1-([1,1′-biphenyl]-4- yl)ethylidene)benzohydrazide |
| I-23 | 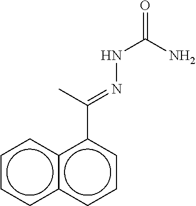 | (E)-2-(1-(naphthalen-1- yl)ethylidene)hydrazine-1-carboxamide |
| I-24 | 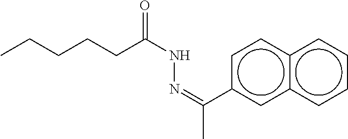 | (Z)-N′-(1-(naphthalen-2- yl)ethylidene)hexanehydrazide |
| I-25 | 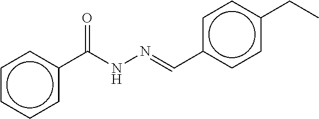 | (E)-N′-(4-ethylbenzylidene)benzohydrazide |
| I-26 | 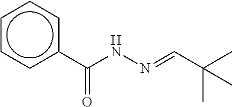 | (E)-N′-(2,2- dimethylpropylidene)benzohydrazide |
| I-27 | 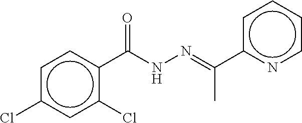 | (E)-2,4-dichloro-N′-(1-(pyridin-2- yl)ethylidene)benzohydrazide |
| I-28 | 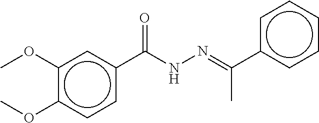 | (E)-3,4-dimethoxy-N′-(1-(pyridin-2- yl)ethylidene)benzohydrazide |
| I-29 | 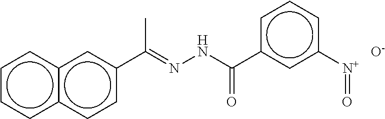 | (E)-N′-(1-(naphthalen-2-yl)ethylidene)-3- nitrobenzohydrazide |
| I-30 | 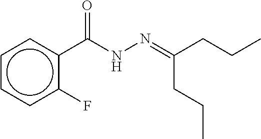 | 2-fluoro-N′-(heptan-4-ylidene)benzohydrazide |
| I-31 | 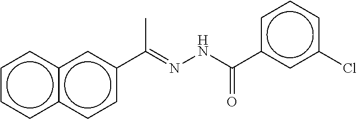 | (E)-3-chloro-N′-(1-(naphthalen-2- yl)ethylidene)benzohydrazide |
| I-32 | 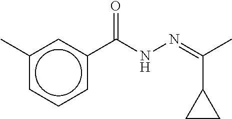 | (Z)-N′-(1-cyclopropylethylidene)-3- methylbenzohydrazide |
| I-33 | 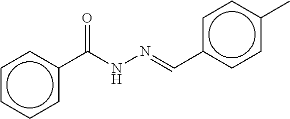 | (E)-N′-(4-methylbenzylidene)benzohydrazide |
| I-34 | 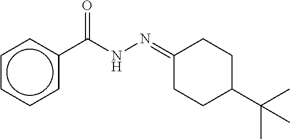 | N′-(4-(tert- butyl)cyclohexylidene)benzohydrazide |
| I-35 | 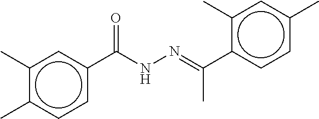 | (E)-N′-(1-(2,4-dimethylphenyl)ethylidene)- 3,4-dimethylbenzohydrazide |
| I-36 | 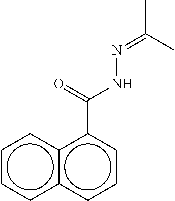 | N′-(propan-2-ylidene)-1-naphthohydrazide |
| I-37 | 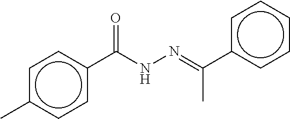 | (E)-4-methyl-N′-(1- phenylethylidene)benzohydrazide |
| I-38 | 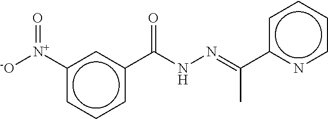 | (E)-3-nitro-N′-(1-(pyridin-2- yl)ethylidene)benzohydrazide |
| I-39 | 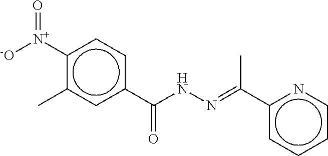 | (E)-3-methyl-4-nitro-N′-(1-(pyridin-2- yl)ethylidene)benzohydrazide |
| I-40 | 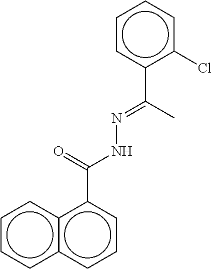 | (E)-N′-(1-(2-chlorophenyl)ethylidene)-1- naphthohydrazide |
| I-41 | 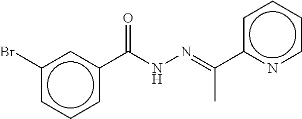 | (E)-3-bromo-N′-(1-(pyridin-2- yl)ethylidene)benzohydrazide |
| I-42 | 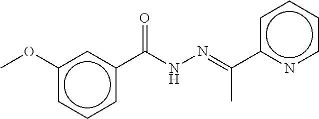 | (E)-3-methoxy-N′-(1-(pyridin-2- yl)ethylidene)benzohydrazide |
| I-43 | 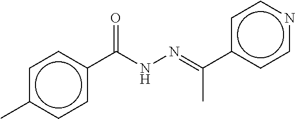 | (E)-4-methyl-N′-(1-(pyridin-4- yl)ethylidene)benzohydrazide |
| I-44 | 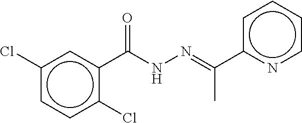 | (E)-2,5-dichloro-N′-(1-(pyridin-2- yl)ethylidene)benzohydrazide |
| I-45 | 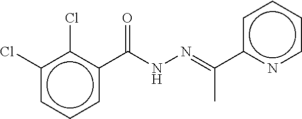 | (E)-2,3-dichloro-N′-(1-(pyridin-2- yl)ethylidene)benzohydrazide |
| I-46 | 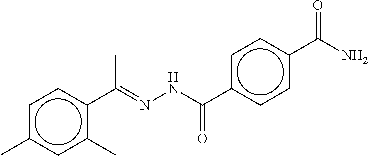 | (E)-4-(2-(1-(2,4- dimethylphenyl)ethylidene)hydrazine-1- carbonyl)benzamide |
| I-47 | 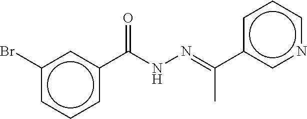 | (E)-3-bromo-N′-(1-(pyridin-3- yl)ethylidene)benzohydrazide |
| I-48 | 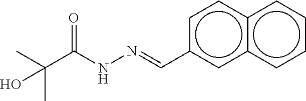 | (E)-2-hydroxy-2-methyl-N′-(naphthalen-2- ylmethylene)propanehydrazide |
| I-49 | 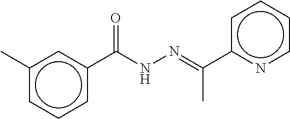 | (E)-3-methyl-N′-(1-(pyridin-2- y)ethylidene)benzohydrazide |
| I-50 | 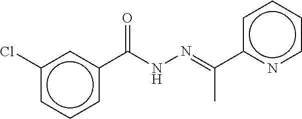 | (E)-3-chloro-N′-(1-(pyridin-2- yl)ethylidene)benzohydrazide |
| I-51 | 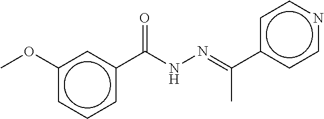 | (E)-3-methoxy-N′-(1-(pyridin-4- yl)ethylidene)benzohydrazide |
| I-52 | 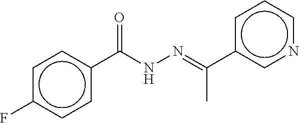 | (E)-4-fluoro-N′-(1-(pyridin-3- yl)ethylidene)benzohydrazide |
| I-53 | 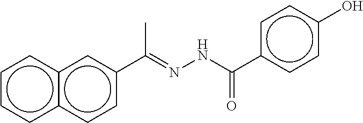 | (E)-4-hydroxy-N′-(1-(naphthalen-2- yl)ethylidene)benzohydrazide |
| I-54 | 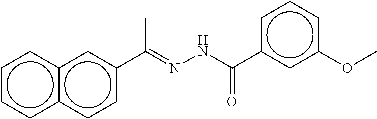 | (E)-3-methoxy-N′-(1-(naphthalen-2- yl)ethylidene)benzohydrazide |
| I-55 | 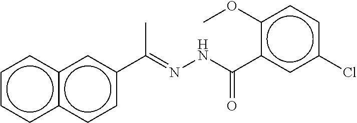 | (E)-5-chloro-2-methoxy-N′-(1-(naphthalen-2- yl)ethylidene)benzohydrazide |
| I-56 | 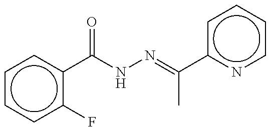 | (E)-2-fluoro-N′-(1-(pyridin-2- yl)ethylidene)benzohydrazide |
| I-57 | 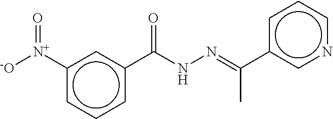 | (E)-3-nitro-N′-(1-(pyridin-3- yl)ethylidene)benzohydrazide |
| I-58 | 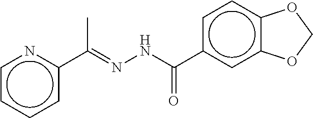 | (E)-N′-(1-(pyridin-2- yl)ethylidene)benzo[d][1,3]dioxole-5- carbohydrazide |
| I-59 | 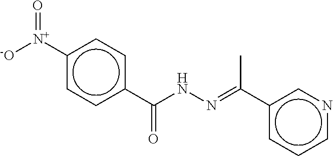 | (E)-4-nitro-N′-(1-(pyridin-3- yl)ethylidene)benzohydrazide |
| I-60 | 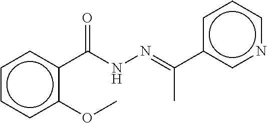 | (E)-2-methoxy-N′-(1-(pyridin-3- yl)ethylidene)benzohydrazide |
| I-61 | 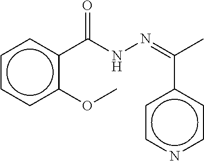 | (Z)-2-methoxy-N′-(1-(pyridin-4- yl)ethylidene)benzohydrazide |
| I-62 | 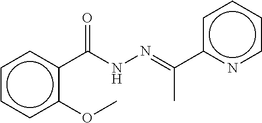 | (E)-2-methoxy-N′-(1-(pyridin-2- yl)ethylidene)benzohydrazide |
| I-63 | 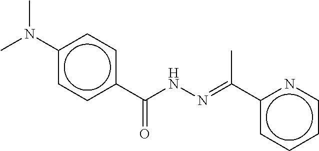 | (E)-4-(dimethylamino)-N′-(1-(pyridin-2- yl)ethylidene)benzohydrazide |
| I-64 | 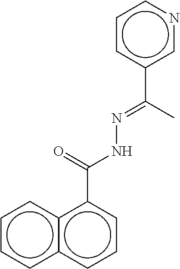 | (E)-N′-(1-(pyriidn-3-yl)ethylidene)-1- naphthohydrazide |
| I-65 | 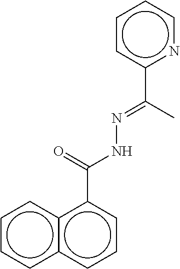 | (E)-N′-(1-(pyridin-2-yl)ethylidene)-1- naphthohydrazide |
| I-66 | 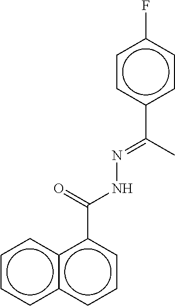 | (E)-N′-(1-(4-fluorophenyl)ethylidene)-1- naphthohydrazide |
| I-67 | 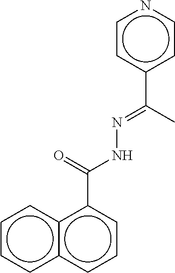 | (E)-N′-(1-(pyridin-4-yl)ethylidene)-1- naphthohydrazide |
| I-68 | 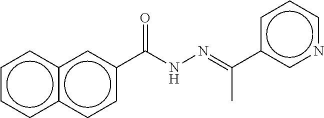 | (E)-N′-(1-(pyridin-3-yl)ethylidene)-2- naphthohydrazide |
| I-69 | 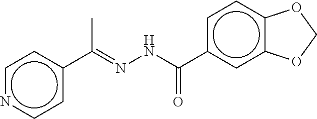 | (E)-N′-(1-(pyridin-4- yl)ethylidene)benzo[d][1,3]dioxole-5- carbohydrazide |
| I-70 | 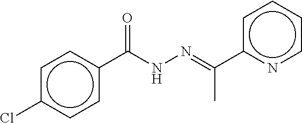 | (E)-4-chloro-N′-(1-(pyridin-2- yl)ethylidene)benzohydrazide |
| I-71 | 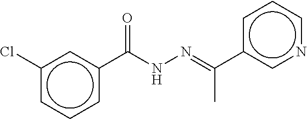 | (E)-3-chloro-N′-(1-(pyridin-3- yl)ethylidene)benzohydrazide |
| I-72 | 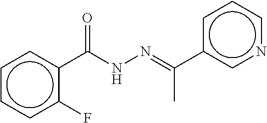 | (E)-2-fluoro-N′-(1-(pyridin-3- yl)ethylidene)benzohydrazide |
| I-73 | 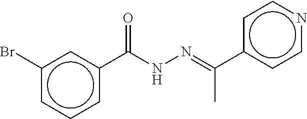 | (E)-3-bromo-N′-(1-(pyridin-4- yl)ethylidene)benzohydrazide |
| I-74 | 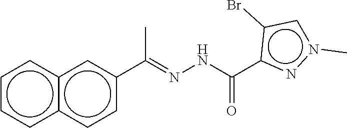 | (E)-4-bromo-1-methyl-N′-(1-(naphthalen-2- yl)ethylidene)-1H-pyrazole-3-carbohydrazide |
| I-75 | 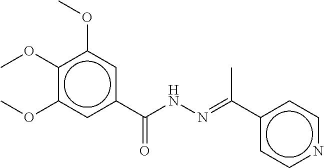 | (E)-3,4,5-trimethoxy-N′-(1-(pyridin-4- yl)ethylidene)benzohydrazide |
| I-76 | 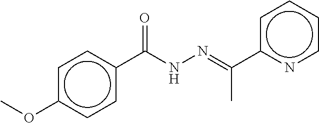 | (E)-4-methoxy-N′-(1-(pyridin-2- yl)ethylidene)benzohydrazide |
| I-77 | 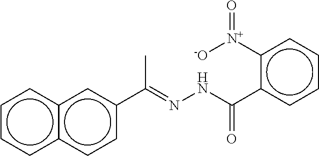 | (E)-N′-(1-(naphthalen-2-yl)ethylidene)-2- nitrobenzohydrazide |
| I-78 | 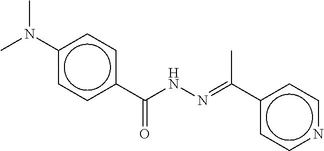 | (E)-4-(dimethylamino)-N′-(1-(pyridin-4- yl)ethylidene)benzohydrazide |
| I-79 | 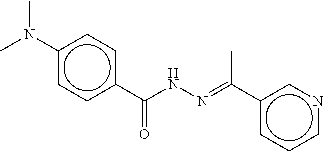 | (E)-4-(dimethylamino)-N′-(1-(pyridin-3- yl)ethylidene)benzohydrazide |
| I-80 | 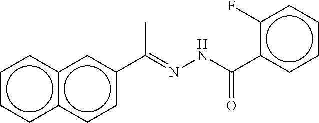 | (E)-2-fluoro-N′-(1-(naphthalen-2- yl)ethylidene)benzohydrazide |
| I-81 | 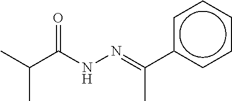 | (E)-N′-(1- phenylethylidene)isobutyrohydrazide |
| I-82 | 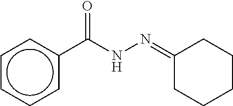 | N′-cyclohexylidenebenzohydrazide |
| I-83 | 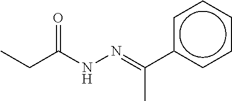 | (E)-N′-(1- phenylethylidene)propionohydrazide |
| I-84 | 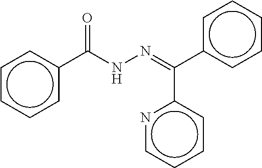 | (Z)-N′-(phenyl(pyridin-2- yl)methylene)benzohydrazide |
| I-85 | 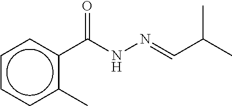 | (E)-2-methyl-N′-(2- methylpropylidene)benzohydrazide |
| I-86 | 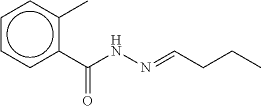 | (E)-N′-butylidene-2-methylbenzohydrazide |
| I-87 | 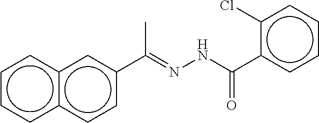 | (E)-2-chloro-N′-(1-(naphthalen-2- yl)ethylidene)benzohydrazide |
| I-88 | 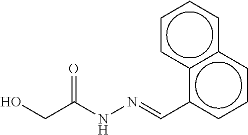 | (E)-2-hydroxy-N′-(naphthalen-1- ylmethylene)acetohydrazide |
| I-89 | 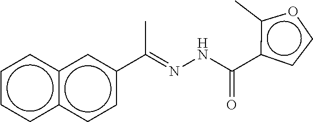 | (E)-2-methyl-N′-(1-(naphthalen-2- yl)ethylidene)furan-3-carbohydrazide |
| I-90 | 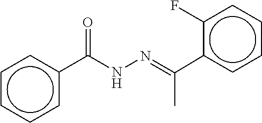 | (E)-N′-(1-(2- fluorophenyl)ethylidene)benzohydrazide |
| I-91 | 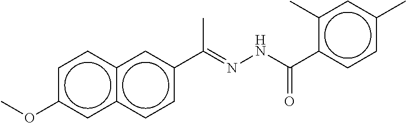 | (E)-N′-(1-(6-methoxynaphthalen-2- yl)ethylidene)-2,4-dimethylbenzohydrazide |
| I-92 | 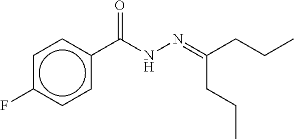 | 4-fluoro-N′-(heptan-4-ylidene)benzohydrazide |
| I-93 | 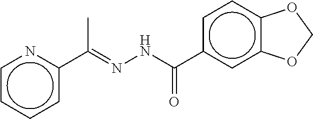 | (E)-N′-(1-(pyridin-2- yl)ethylidene)benzo[d][1,3]dioxole-5- carbohydrazide |
| I-94 | 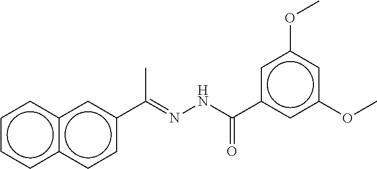 | (E)-3,5-dimethoxy-N′-(1-(naphthalen-2- yl)ethylidene)benzohydrazide |
| I-95 | 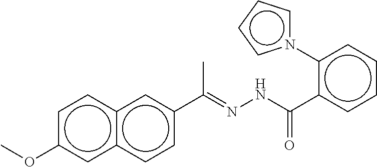 | (E)-N′-(1-(6-methoxynaphthalen-2- yl)ethylidene)-2-(1H-pyrrol-1- yl)benzohydrazide |
| I-96 | 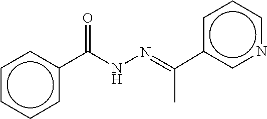 | (E)-N′-(1-(pyridin-3- yl)ethylidene)benzohydrazide |
| I-97 | 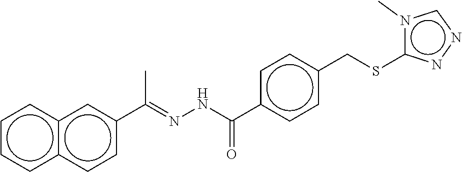 | (E)-4-(((4-methyl-4H-1,2,4-triazol-3- yl)thio)methyl)-N′-(1-(naphthalen-2- yl)ethylidene)benzohydrazide |
| I-98 | 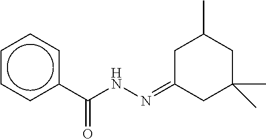 | (E)-N′-(3,3,5- trimethylcyclohexylidene)benzohydrazide |
| I-99 | 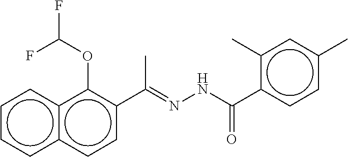 | (E)-N′-(1-(1-(difluoromethoxy)naphthalen-2- yl)ethylidene)-2,4-dimethylbenzohydrazide |
| I-100 | 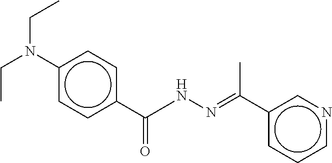 | (E)-4-(diethylamino)-N′-(1-(pyriidn-3- yl)ethylidene)benzohydrazide |
| I-101 | 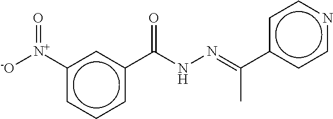 | (E)-3-nitro-N′-(1-(pyridin-4- yl)ethylidene)benzohydrazide |
| I-102 | 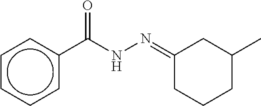 | (E)-N′-(3- methylcyclohexylidene)benzohydrazide |
| I-103 | 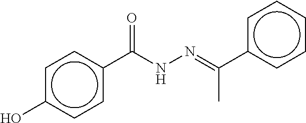 | (E)-4-hydroxy-N′-(1- phenylethylidene)benzohydrazide |
| I-104 | 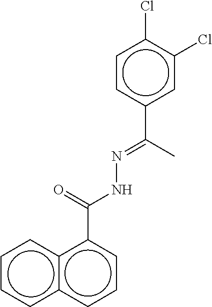 | (E)-N′-(1-(3,4-dichlorophenyl)ethylidene)-1- naphthohydrazide |
| I-105 | 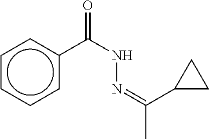 | (Z)-N′-(1- cyclopropylethylidene)benzohydrazide |
| I-106 | 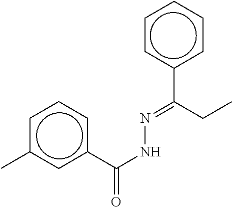 | (E)-3-methyl-N′-(1- phenylpropylidene)benzohydrazide |
| I-107 | 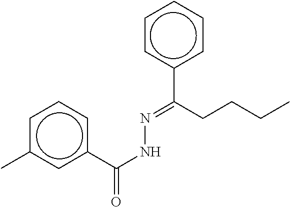 | (E)-3-methyl-N′-(1- phenylpentylidene)benzohydrazide |
| I-108 | 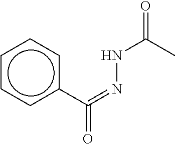 | (Z)-N′-(1-phenylethylidene)acetohydrazide |
| I-109 | 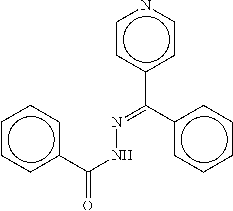 | (E)-N′-(phenyl(pyridin-4- yl)methylene)benzohydrazide |
| I-110 | 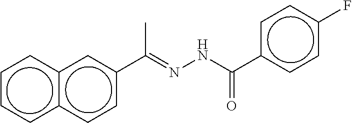 | (E)-4-fluoro-N′-(1-(naphthalen-2- yl)ethylidene)benzohydrazide |
| I-111 | 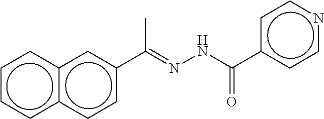 | (E)-N′-(1-(naphthalen-2- yl)ethylidene)isonicotinohydrazide |
| I-112 | 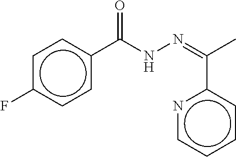 | (Z)-4-fluoro-N′-(1-(pyridin-2- yl)ethylidene)benzohydrazide |
| I-113 | 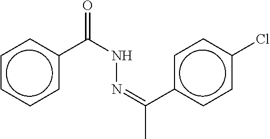 | (Z)-N′-(1-(4- chlorophenyl)ethylidene)benzohydrazide |
| I-114 | 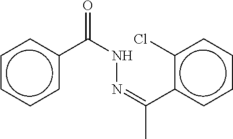 | (Z)-N′-(1-(2- chlorophenyl)ethylidene)benzohydrazide |
| I-115 | 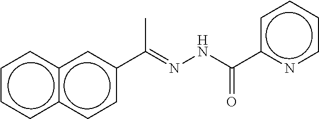 | (E)-N′-(1-(naphthalen-2- yl)ethylidene)picolinohydrazide |
| I-116 | 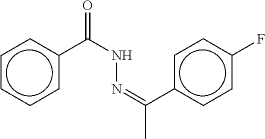 | (Z)-N′-(1-(4- fluorophenyl)ethylidene)benzohydrazide |
| I-117 | 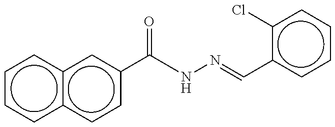 | (E)-N′-(2-chlorobenzylidene)-2- naphthohydrazide |
| I-118 | 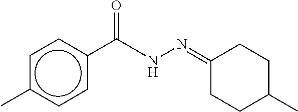 | 4-methyl-N′-(4- methylcyclohexylidene)benzohydrazide |
| I-119 | 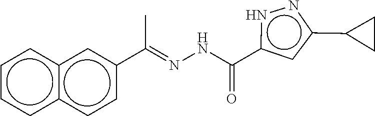 | (E)-3-cyclopropyl-N′-(1-(naphthalen-2- yl)ethylidene)-1H-pyrazole-5-carbohydrazide |
| I-120 | 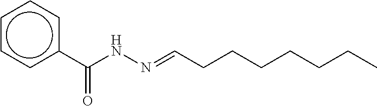 | (E)-N′-octylidenebenzohydrazide |
| I-121 | 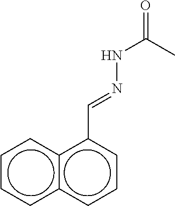 | (E)-N′-(naphthalen-1- ylmethylene)acetohydrazide |
| I-122 | 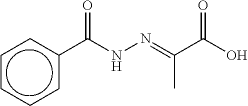 | (E)-2-(2-benzoylhydrazineylidene)propanoic acid |
| I-123 | 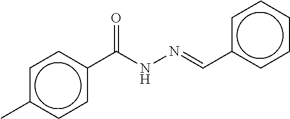 | (E)-N′-benzylidene-4-methylbenzohydrazide |
| I-124 | 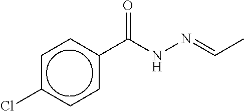 | (E)-4-chloro-N′-ethylidenebenzohydrazide |
| I-125 | 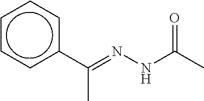 | (E)-N′-(1-phenylethylidene)acetohydrazide |
| I-126 | 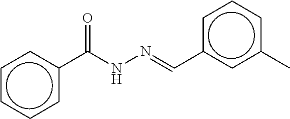 | (E)-N′-(3-methylbenzylidene)benzohydrazide |
| I-127 | 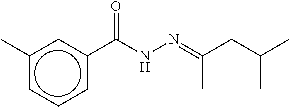 | (E)-3-methyl-N′-(4-methylpentan-2- ylidene)benzohydrazide |
| I-128 | 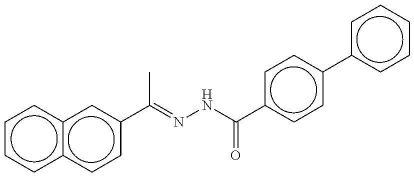 | (E)-N′-(1-(naphthalen-2-yl)ethylidene)-[1,1′- biphenyl]-4-carbohydrazide |
| I-129 | 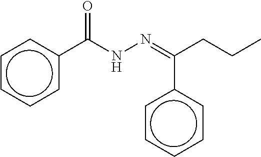 | (Z)-N′-(1-phenylbutylidene)benzohydrazide |
| I-130 | 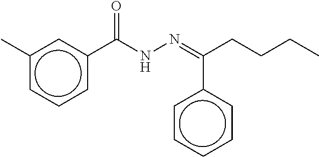 | (Z)-3-methyl-N′-(1- phenylpentylidene)benzohydrazide |
| I-131 | 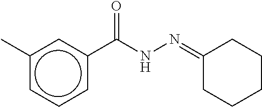 | N′-cyclohexylidene-3-methylbenzohydrazide |
| I-132 | 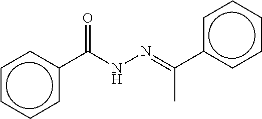 | (E)-N′-(1-phenylethylidene)benzohydrazide |
| I-133 | 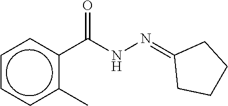 | N′-cyclopentylidene-2-methylbenzohydrazide |
| I-134 | 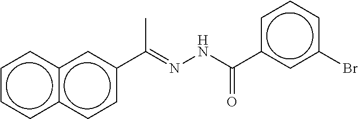 | (E)-3-bromo-N′-(1-(naphthalen-2- yl)ethylidene)benzohydrazide |
| I-135 | 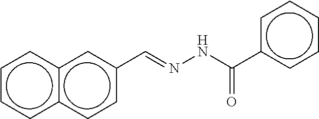 | (E)-N′-(naphthalen-2- ylmethylene)benzohydrazide |
| I-136 | 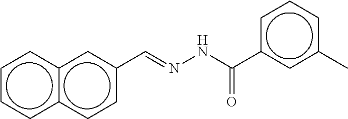 | (E)-3-methyl-N′-(naphthalen-2- ylmethylene)benzohydrazide |
| I-137 | 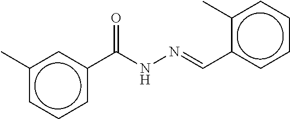 | (E)-3-methyl-N′-(2- methylbenzylidene)benzohydrazide |
| I-138 | 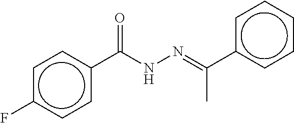 | (E)-4-fluoro-N′-(1- phenylethylidene)benzohydrazide |
| I-139 | 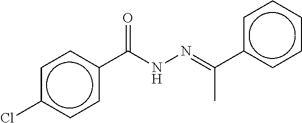 | (E)-4-chloro-N′-(1- phenylethylidene)benzohydrazide |
| I-140 | 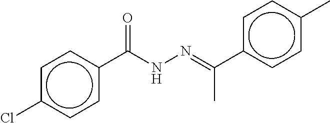 | (E)-4-chloro-N′-(1-(p- tolyl)ethylidene)benzohydrazide |
| I-141 | 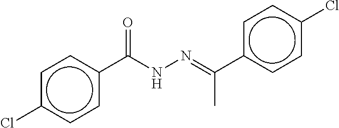 | (E)-4-chloro-N′-(1-(4- chlorophenyl)ethylidene)benzohydrazide |
| I-142 | 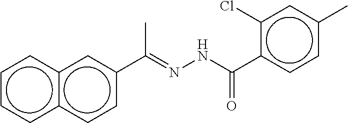 | (E)-2-chloro-4-methyl-N′-(1-(naphthalen-2- yl)ethylidene)benzohydrazide |
| I-143 | 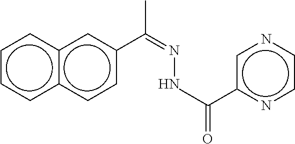 | (Z)-N′-(1-(naphthalen-2- yl)ethylidene)pyrazine-2-carbohydrazide |
| I-144 | 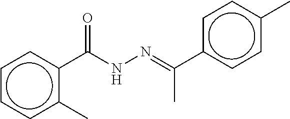 | (E)-2-methyl-N′-(1-(p- tolyl)ethylidene)benzohydrazide |
| I-145 | 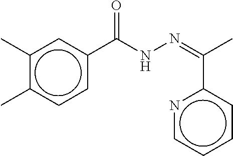 | (Z)-3,4-dimethyl-N′-(1-(pyridin-2- yl)ethylidene)benzohydrazide |
| I-146 | 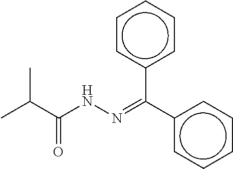 | N′-(diphenylmethylene)isobutyrohydrazide |
| I-147 | 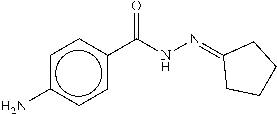 | 4-amino-N′-cyclopentylidenebenzohydrazide |
| I-148 | 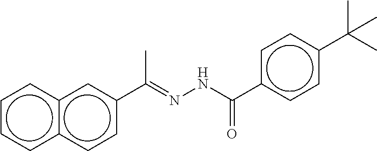 | (E)-4-(tert-butyl)-N′-(1-(naphthalen-2- yl)ethylidene)benzohydrazide |
| I-149 | 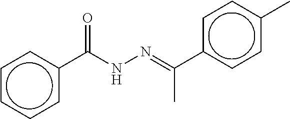 | (E)-N′-(1-(p-tolyl)ethylidene)benzohydrazide |
| I-150 | 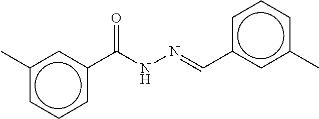 | (E)-3-methyl-N′-(3- methylbenzylidene)benzohydrazide |
| I-151 | 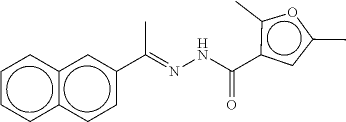 | (E)-2,5-dimethyl-N′-(1-(naphthalen-2- yl)ethylidene)furan-3-carbohydrazide |
| I-152 | 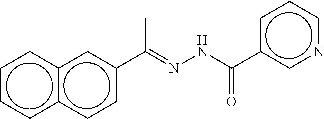 | (E)-N′-(1-(naphthalen-2- yl)ethylidene)nicotinohydrazide |
| I-153 | 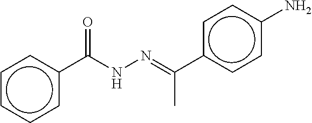 | (E)-N′-(1-(4- aminophenyl)ethylidene)benzohydrazide |
| I-154 | 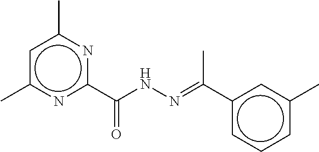 | (E)-4,6-dimethyl-N′-(1- phenylethylidene)pyrimidine-2- carbohydrazide |
| I-155 | 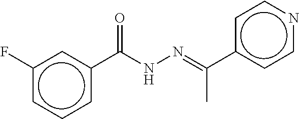 | (E)-3-fluoro-N′-(1-(pyriidn-4- yl)ethylidene)benzohydrazide |
| I-156 | 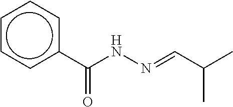 | (E)-N′-(2-methylpropylidene)benzohydrazide |
| I-157 | 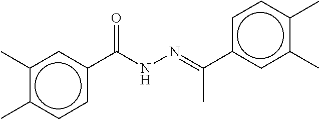 | (E)-N′-(1-(3,4-dimethylphenyl)ethylidene)- 3,4-dimethylbenzohydrazide |
| I-158 | 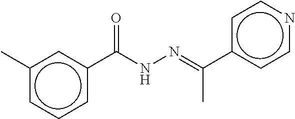 | (E)-3-methyl-N′-(1-(pyridin-4- yl)ethylidene)benzohydrazide |
| I-159 | 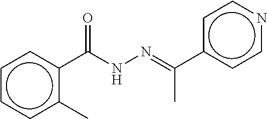 | (E)-2-methyl-N′-(1-(pyridin-4- yl)ethylidene)benzohydrazide |
| I-160 | 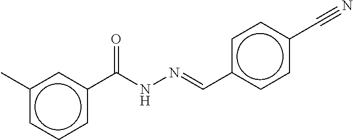 | (E)-N′-(4-cyanobenzylidene)-3- methylbenzohydrazide |
| I-161 | 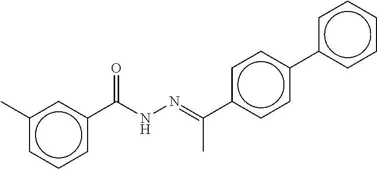 | (E)-N′-(1-([1,1′-biphenyl]-4-yl)ethylidene)-3- methylbenzohydrazide |
| I-162 | 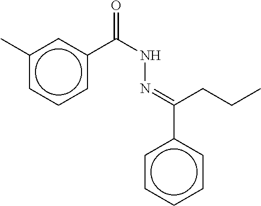 | (E)-3-methyl-N′-(1- phenylbutylidene)benzohydrazide |
| I-163 | 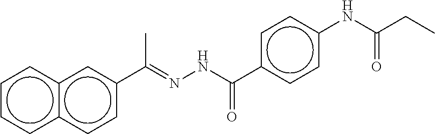 | (E)-N-(4-(2-(1-(naphthalen-2- yl)ethylidene)hydrazine-1- carbonyl)phenyl)propionamide |
| I-164 | 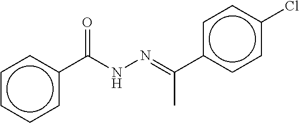 | (E)-N′-(1-(4- chlorophenyl)ethylidene)benzohydrazide |
| I-165 | 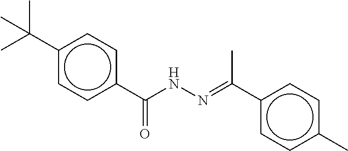 | (E)-4-(tert-butyl)-N′-(1-(p- tolyl)ethylidene)benzohydrazide |
| I-166 | 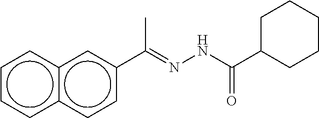 | (E)-N′-(1-(naphthalen-2- yl)ethylidene)cyclohexanecarbohydrazide |
| I-167 | 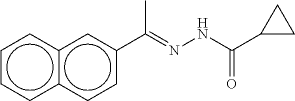 | (E)-N′-(1-(naphthalen-2- yl)ethylidene)cyclopropanecarbohydrazide |
| I-168 | 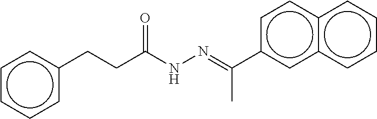 | (E)-N′-(1-(naphthalen-2-yl)ethylidene)-3- phenylpropanehydrazide |
| I-169 | 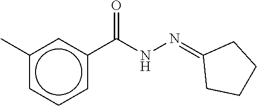 | N′-cyclopentylidene-3-methylbenzohydrazide |
| I-170 | 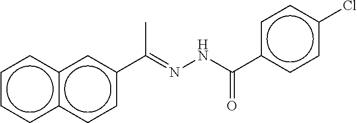 | (E)-4-chloro-N′-(1-(naphthalen-2- yl)ethylidene)benzohydrazide |
| I-171 | 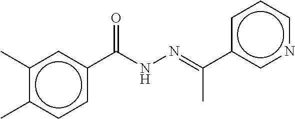 | (E)-3,4-dimethyl-N′-(1-(pyridin-3- yl)ethylidene)benzohydrazide |
| I-172 | 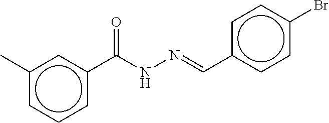 | (E)-N′-(4-bromobenzylidene)-3- methylbenzohydrazide |
| I-173 | 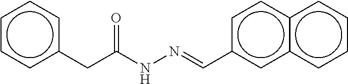 | (E)-N′-(naphthalen-1-ylmethylene)-2- phenylacetohydrazide |
| I-174 | 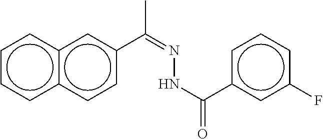 | (Z)-3-fluoro-N′-(1-(naphthalen-2- yl)ethylidene)benzohydrazide |
| I-175 | 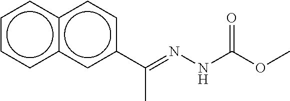 | methyl (E)-2-(1-(naphthalen-2- yl)ethylidene)hydrazine-1-carboxylate |
| I-176 | 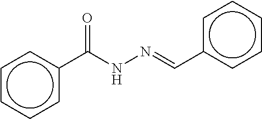 | (E)-N′-benzylidenebenzohydrazide |
| I-177 | 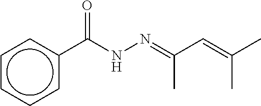 | (E)-N′-(4-methylpent-3-en-2- ylidene)benzohydrazide |
| I-178 | 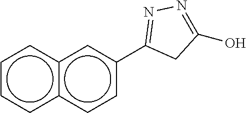 | 5-(naphthalen-2-yl)-4H-pyrazol-3-ol |
| I-179 | 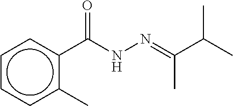 | (E)-2-methyl-N′-(3-methylbutan-2- ylidene)benzohydrazide |
| I-180 | 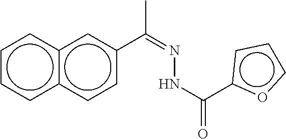 | (Z)-N′-(1-(naphthalen-2-yl)ethylidene)furan- 2-carbohydrazide |
| I-181 | 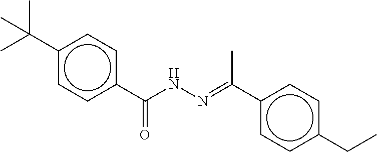 | (E)-4-(tert-butyl)-N′-(1-(4- ethylphenyl)ethylidene)benzohydrazide |
| I-182 | 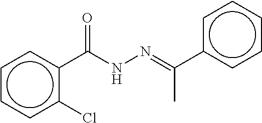 | (E)-2-chloro-N′-(1- phenylethylidene)benzohydrazide |
| I-183 | 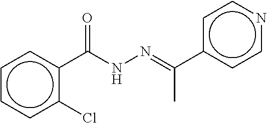 | (E)-2-chloro-N′-(1-(pyridin-4- yl)ethylidene)benzohydrazide |
| I-184 | 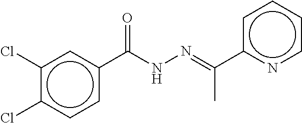 | (E)-3,4-dichloro-N′-(1-(pyridin-2- yl)ethylidene)benzohydrazide |
| I-185 | 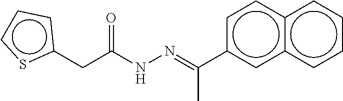 | (E)-N′-(1-(naphthalen-2-yl)ethylidene)-2- (thiophen-2-yl)acetohydrazide |
| I-186 | 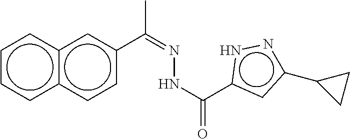 | (Z)-3-cyclopropyl-N′-(1-(naphthalen-2- yl)ethylidene)-1H-pyrazole-5-carbohydrazide |
| I-187 | 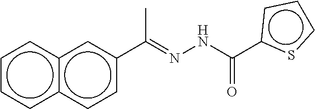 | (E)-N′-(1-(naphthalen-2- yl)ethylidene)thiophene-2-carbohydrazide |
| I-188 | 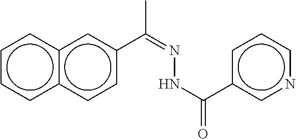 | (Z)-N′-(1-(naphthalen-2- yl)ethylidene)nicotinohydrazide |
| I-189 | 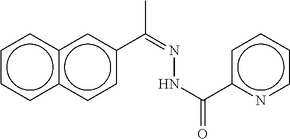 | (Z)-N′-(1-(naphthalen-2- yl)ethylidene)picolinohydrazide |
| I-190 | 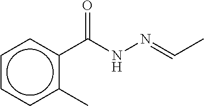 | (E)-N′-ethylidene-2-methylbenzohydrazide |
| I-191 | 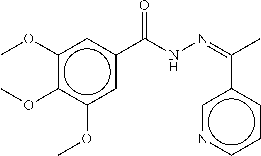 | (Z)-3,4,5-trimethoxy-N′-(1-(pyridin-3- yl)ethylidene)benzohydrazide |
| I-192 | 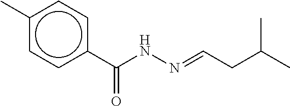 | (E)-4-methyl-N′-(3- methylbutylidene)benzohydrazide |
| I-193 | 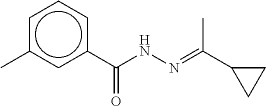 | (E)-N′-(1-cyclopropylethylidene)-3- methylbenzohydrazide |
| I-194 | 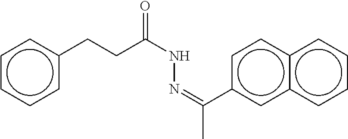 | (Z)-N′-(1-(naphthalen-2-yl)ethylidene)-3- phenylpropanehydrazide |
| I-195 | 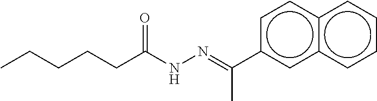 | (E)-N′-(1-(naphthalen-2- yl)ethylidene)hexanehydrazide |
| I-196 | 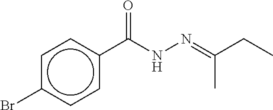 | (E)-4-bromo-N′-(butan-2- ylidene)benzohydrazide |
| I-197 | 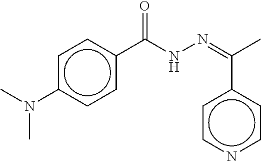 | (Z)-4-(dimethylamino)-N′-(1-(pyridin-4- yl)ethylidene)benzohydrazide |
| I-198 | 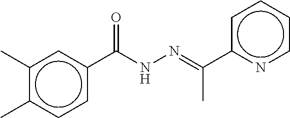 | (E)-3,4-dimethyl-N′-(1-(pyridin-2- yl)ethylidene)benzohydrazide |
| I-199 | 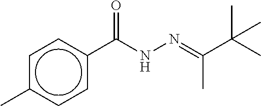 | (E)-N′-(3,3-dimethylbutan-2-ylidene)-4- methylbenzohydrazide |
| I-200 | 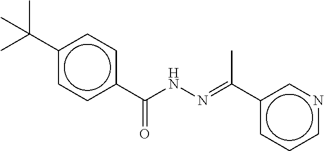 | (E)-4-(tert-butyl)-N′-(1-(pyridin-3- yl)ethylidene)benzohydrazide |
| I-201 | 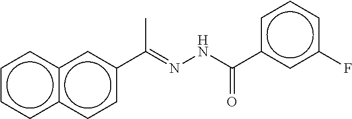 | (E)-3-fluoro-N′-(1-(naphthalen-2- yl)ethylidene)benzohydrazide |
| I-202 | 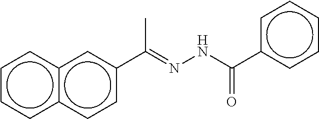 | (E)-N′-(1-(naphthalen-2- yl)ethylidene)benzohydrazide |
| I-203 | 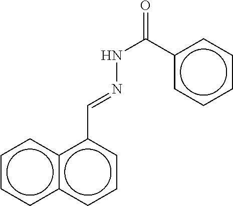 | (E)-N′-(naphthalen-1- ylmethylene)benzohydrazide |
| I-204 | 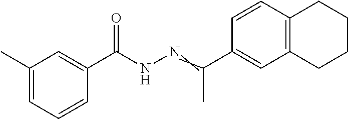 [SMILES: CC(=NNC(=O)c1cccc(C)c1)c1ccc2c(c1)CCCC2] | (E)-3-methyl-N′-(1-(5,6,7,8- tetrahydronapthalen-2- yl)ethylidene)benzohydrazide |
| I-205 | 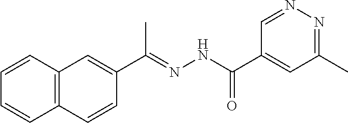 [SMILES: C/C(=N\NC(=O)c1cnnc(C)c1)c1ccc2ccccc2c1] | (E)-6-methyl-N′-(1-(naphthalen-2- yl)ethylidene)pyridazine-4-carbohydrazide |
| I-206 | 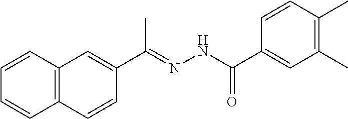 [SMILES: C/C(=N\NC(=O)c1ccc(C)c(C)c1)c1ccc2ccccc2c1] | (E)-3,4-dimethyl-N′-(1-(naphthalen-2- yl)ethylidene)benzohydrazide |
| I-207 | 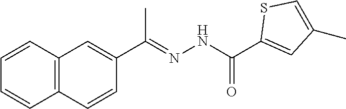 [SMILES: C/C(=N\NC(=O)c1cc(C)cs1)c1ccc2ccccc2c1] | (E)-4-methyl-N′-(1-(naphthalen-2- yl)ethylidene)thiophene-2-carbohydrazide |
| I-208 | 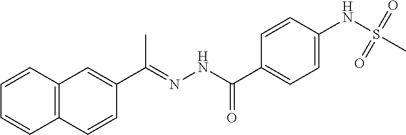 [SMILES: C/C(=N\NC(=O)c1ccc(NS(C)(=O)=O)cc1)c1ccc2ccccc2c1] | (E)-N-(4-(2-(1-(naphthalen-2- yl)ethylidene)hydrazine-1- carbonyl)phenyl)methanesulfonamide |
| I-209 | 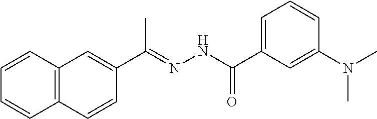 [SMILES: C/C(=N\NC(=O)c1cccc(N(C)C)c1)c1ccc2ccccc2c1] | (E)-3-(dimethylamino)-N′-(1-(naphthalen-2- yl)ethylidene)benzohydrazide |
| I-210 | 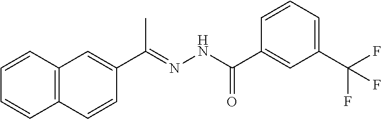 [SMILES: C/C(=N\NC(=O)c1cccc(C(F)(F)F)c1)c1ccc2ccccc2c1] | (E)-N′-(1-(naphthalen-2-yl)ethylidene)-3- (trifluoromethyl)benzohydrazide |
| I-211 | 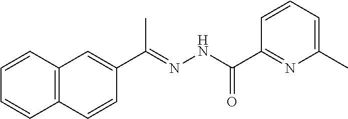 [SMILES: C/C(=N\NC(=O)c1cccc(C)n1)c1ccc2ccccc2c1] | (E)-6-methyl-N′-(1-(naphthalen-2- yl)ethylidene)picolinohydrazide |
| I-212 | 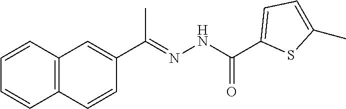 [SMILES: C/C(=N\NC(=O)c1ccc(C)s1)c1ccc2ccccc2c1] | (E)-5-methyl-N′-(1-(naphthalen-2- yl)ethylidene)thiophene-2-carbohydrazide |
| I-213 | 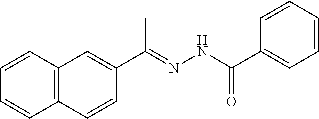 [SMILES: C/C(=N\NC(=O)c1ccccc1)c1ccc2ccccc2c1] | (E)-N′-(1-(naphthalen-2- yl)ethylidene)benzohydrazide |
| I-214 | 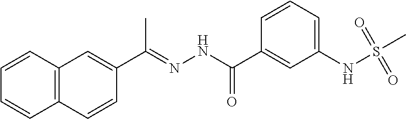 [SMILES: C/C(=N\NC(=O)c1cccc(NS(C)(=O)=O)c1)c1ccc2ccccc2c1] | (E)-N-(3-(2-(1-(naphthalen-2- yl)ethylidene)hydrazine-1- carobnyl)phenyl)methanesulfonamide |
| I-215 | 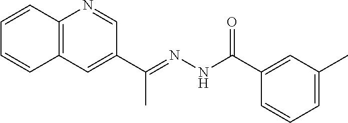 [SMILES: C/C(=N\NC(=O)c1cccc(C)c1)c1cnc2ccccc2c1] | (E)-3-methyl-N′-(1-(quinolin-3- yl)ethylidene)benzohydrazide |
| I-216 | 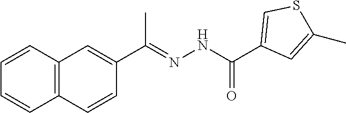 [SMILES: C/C(=N\NC(=O)c1csc(C)c1)c1ccc2ccccc2c1] | (E)-5-methyl-N′-(1-(naphthalen-2- yl)ethylidene)thiophene-3-carbohydrazidee |
| I-217 | 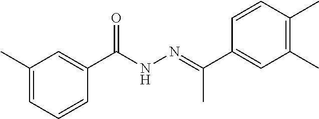 | (E)-N′-(1-(3,4-dimethylphenyl)ethylidene)-3- methylbenzohydrazide |
| I-218 | 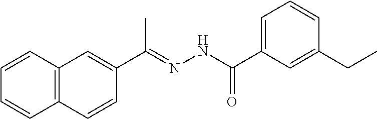 [SMILES: CCc1cccc(C(=O)N/N=C(\C)c2ccc3ccccc3c2)c1] | (E)-3-ethyl-N′-(1-(naphthalen-2- yl)ethylidene)benzohydrazide |
| I-219 | 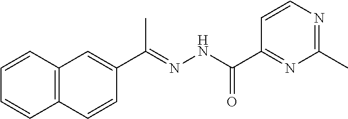 [SMILES: C/C(=N\NC(=O)c1ccnc(C)n1)c1ccc2ccccc2c1] | (E)-2-methyl-N′-(1-(naphthalen-2- yl)ethylidene)pyrimidine-4-carbohydrazide |
| I-220 | 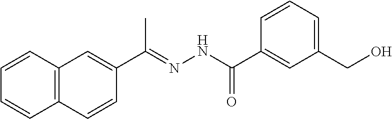 [SMILES: C/C(=N\NC(=O)c1cccc(CO)c1)c1ccc2ccccc2c1] | (E)-3-(hydroxymethyl)-N′-(1-(naphthalen-2- yl)ethylidene)benzohydrazide |
| I-221 | 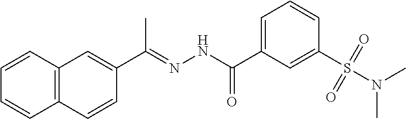 [SMILES: C/C(=N\NC(=O)c1cccc(S(=O)(=O)N(C)C)c1)c1ccc2ccccc2c1] | (E)-N,N-dimethyl-3-(2-(1-(naphthalen-2- yl)ethylidene)hydrazine-1- carbonyl)benzenesulfonamide |
| I-222 | 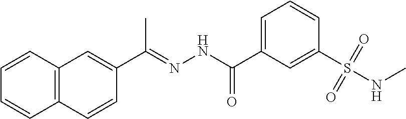 [SMILES: CNS(=O)(=O)c1cccc(C(=O)N/N=C(\C)c2ccc3ccccc3c2)c1] | (E)-N-methyl-3-(2-(1-(naphthalen-2- yl)ethylidene)hydrazine-1- carbonyl)benzenesulfonamide |
| I-223 | 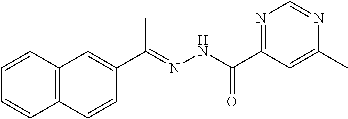 [SMILES: C/C(=N\NC(=O)c1cc(C)ncn1)c1ccc2ccccc2c1] | (E)-6-methyl-N′-(1-(naphthalen-2- yl)ethylidene)pyrimidine-4-carbohydrazide |
| I-224 | 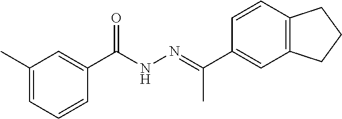 [SMILES: C/C(=N\NC(=O)c1cccc(C)c1)c1ccc2c(c1)CCC2] | (E)-N′-(1-(2,3-dihydro-1H-inden-5- yl)ethylidene)-3-methylbenzohydrazide |
| I-225 | 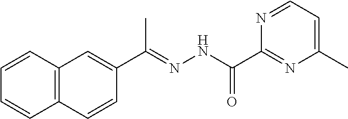 [SMILES: C/C(=N\NC(=O)c1nccc(C)n1)c1ccc2ccccc2c1] | (E)-4-methyl-N′-(1-(naphthalen-2- yl)ethylidene)pyrimidine-2-carbohydrazide |
| I-226 | 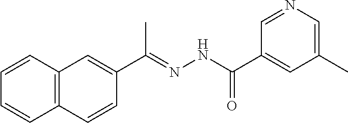 [SMILES: C/C(=N\NC(=O)c1cncc(C)c1)c1ccc2ccccc2c1] | (E)-5-methyl-N′-(1-(naphthalen-2- yl)ethylidene)nicotinohydrazide |
| I-227 | 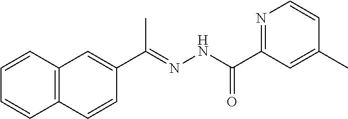 [SMILES: C/C(=N\NC(=O)c1cc(C)ccn1)c1ccc2ccccc2c1] | (E)-4-methyl-N′-(1-(naphthalen-2- yl)ethylidene)picolinohydrazide |
| I-228 | 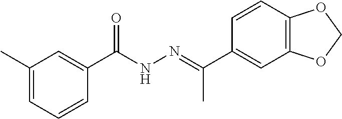 [SMILES: C/C(=N\NC(=O)c1cccc(C)c1)c1ccc2c(c1)OCO2] | (E)-N′-(1-(benzo[d][1,3]dioxol-5- yl)ethylidene)-3-methylbenzohydrazide |
| I-229 | 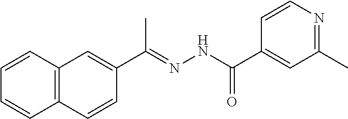 [SMILES: C/C(=N\NC(=O)c1ccnc(C)c1)c1ccc2ccccc2c1] | (E)-2-methyl-N′-(1-(naphthalen-2- yl)ethylidene)isoinicotinohydrazide |
| I-230 | 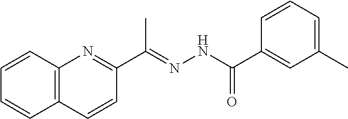 [SMILES: C/C(=N\NC(=O)c1cccc(C)c1)c1ccc2ccccc2n1] | (E)-3-methyl-N′-(1-(quinolin-2- yl)ethylidene)benzohydrazide |
| I-231 | 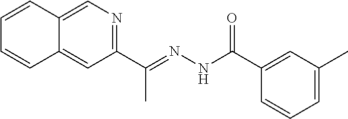 [SMILES: C/C(=N\NC(=O)c1cccc(C)c1)c1cc2ccccc2cn1] | (E)-N′-(1-(isoquinolin-3-yl)ethylidene)-3- methylbenzohydrazide |
| I-232 | 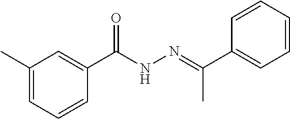 [SMILES: C/C(=N\NC(=O)c1cccc(C)c1)c1ccccc1] | (E)-3-methyl-N′-(1- phenylethylidene)benzohydrazide |
| I-233 | 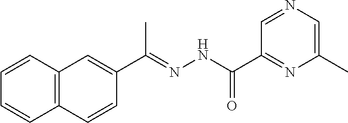 [SMILES: C/C(=N\NC(=O)c1cncc(C)n1)c1ccc2ccccc2c1] | (E)-6-methyl-N′-(1-(naphthalen-2- yl)ethylidene)pyrazine-2-carbohydrazide |
| I-234 | 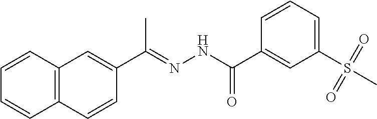 [SMILES: C/C(=N\NC(=O)c1cccc(S(C)(=O)=O)c1)c1ccc2ccccc2c1] | (E)-3-(methylsulfonyl)-N′-(1-(naphthalen-2- yl)ethylidene)benzohydrazide |
| I-235 | 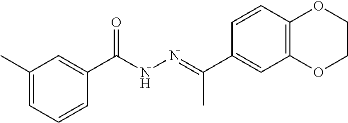 [SMILES: C/C(=N\NC(=O)c1cccc(C)c1)c1ccc2c(c1)OCCO2] | (E)-N′-(1-(2,3-dihydrobenzo[b][1,4]dioxin-6- yl)ethylidene)-3-methylbenzohydrazide |

### III. Methods for Making Compounds

Compounds disclosed herein, including compounds of Formulas (I), (Ia), (Ib), (II), (IIa), (III), (IIIa), (IV), (V), (VI), (VII) and/or (VIII) can be prepared as will be understood by those of skill upon consideration of the present disclosure. For example, such compounds can be prepared by the condensation of an acyl hydrazide with an aldehyde or ketone. In one embodiment, compounds of Formulas (I), (Ia), (Ib), (II), (IIa), (III), (IIIa), (IV), (V), (VI), (VII) and/or (VIII) are prepared according to Scheme (I), illustrated below:

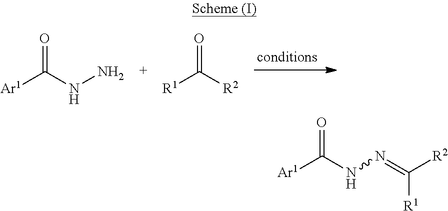

wherein Ar¹, R¹ and R² are selected from those described above in section II. With continued reference to Scheme (I), appropriate conditions can be determined by those of skill in the art, and may include, without limitation, mildly acidic conditions. Exemplary conditions that can be adapted to prepare the present compounds of Formulas (I), (Ia), (Ib), (II), (IIa), (III), (IIIa), (IV), (V), (VI), (VII) and/or (VIII) are described in G Vantomme, S Jiang & J-M Lehn, J. Am. Chem. Soc., 2014, 136, 9509-9518, and K Jasiak & A Kudelko, Tetrahedron Lett., 2015, 56, 5878-5881. Similarly, suitable starting materials, such as acyl hydrazides, can be prepared as is known to those of skill in the art, for example, from esters of the formula Ar¹CO₂R (wherein R is alkyl). Suitable ketones and aldehydes for condensation with acyl hydrazides also can be prepared as is known to those of skill in the art.

### IV. Target Crops and their Pathogens

The present disclosure provides formulations and methods for their use in treating crops for pathogens. In one embodiment, one or more presently disclosed compounds, such as a compound of Formulas (I), (Ia), (Ib), (II), (IIa), (III), (IIIa), (IV), (V), (VI), (VII) and/or (VIII), is administered in combination with an agricultural or horticultural pesticide, such as an acaricide, antimicrobial, fungicide, herbicide, insecticide, molluscicide and/or nematocide. Crops that can be treated, include those plagued by various pathogens, including without limitation, bacteria, viruses, fungal pathogens, mites, nematodes, molluscs, weeds or other pests, as is known to those of ordinary skill in the agricultural arts. By way of example, such agricultural and horticultural crops that can be treated according to the present disclosure include plants, whether genetically modified or not, including their harvested products, such as: cereals; vegetables; root crops; potatoes; trees such as fruit trees, for example banana trees, tea, coffee trees, or cocoa trees; grasses; lawn grass; or cotton.

Roux and coworkers describe the compound:

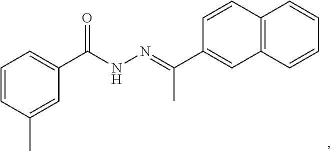 [SMILES: C/C(=N\NC(=O)c1cccc(C)c1)c1ccc2ccccc2c1]

referred to herein as “Roux compound 15,” as enhancing the ability of certain fungicides to inhibit the growth of different plant-pathogenic fungi (Molecular Plant Pathology, 2017, 18(7), 1012-1023; and WO 2016/123191). The present compounds surprisingly enhance the ability of a variety of pesticides against a broad variety of pathogens, including fungal pathogens. In addition, examples of the presently disclosed compound exhibit superior enhancer activity than Roux compound 15.

The agricultural or horticultural enhancer disclosed herein may be applied to each part of plants, such as leaves, stems, patterns, flowers, buds, fruits, seeds, sprouts, roots, tubers, tuberous roots, shoots, or cuttings. The agricultural or horticultural enhancer according to the present disclosure may also be applied to improved varieties/varieties, cultivars, as well as mutants, hybrids and genetically modified embodiments of these plants.

The agricultural or horticultural treatment described herein may be used to conduct seed treatment, foliage application, soil application, or water application, so as to control various diseases occurring in agricultural or horticultural crops, including flowers, lawns, and pastures.

The present compounds are useful for potentiating the effects of antimicrobial agents. For example, the present compounds can be used in combination with an antimicrobial agent to combat bacterial and viral infection.

The present compounds are useful for potentiating the effects of herbicides. For example, the present compounds can be used in combination with one or more herbicide to control weeds or other unwanted vegetation.

The present compounds are useful for potentiating the effects of insecticides. For example, the present compounds can be used in combination with one or more insecticide to control insect infestation.

The present compounds are useful for potentiating the effects of acaricides or miticides. For example, the present compounds can be used in combination with one or more acaricidal agent to control mites.

The present compounds are useful for potentiating the effects of molluscicides. For example, the present compounds can be used in combination with one or more molluscicide to prevent interference of slugs or snails with a crop.

The present compounds are useful for potentiating the effects of nematocides. For example, the present compounds can be used in combination with one or more nematocide to prevent interference of nematodes with a crop.

The present compounds are particularly useful for potentiating the effects of fungicides against plant fungal pathogens. Examples of pathogens treated according to the present disclosure include, without limitation, Botrytis cinerea, Colletotrichum graminicola, Fusarium oxysporum, Sclerotiana sclerotiorum, Verticillium dahlia, Mycosphaerella graminicola and Sphacelotheca reliana.

Botrytis cinerea is an airborne plant pathogen with a necrotrophic lifestyle attacking over 200 crop hosts worldwide. It mainly attacks dicotyledonous plant species, including important protein, oil, fiber and horticultural crops, grapes and strawberries and also Botrytis also causes secondary soft rot of fruits and vegetables during storage, transit and at the market. Many classes of fungicides have failed to control Botrytis cinerea due to its genetic plasticity.

The genus Colletotrichum comprises ˜600 species attacking over 3,200 species of monocot and dicot plants. Colletotrichum graminicola primarily infects maize (Zea mays), causing annual losses of approximately 1 billion dollars in the United States alone (Connell et al., 2012).

Fusarium wilt of banana, caused by the soil-borne fungus Fusarium oxysporum f. sp. cubense, is a major threat to banana production worldwide. No fungicides are currently available to effectively control the disease once plants are infected (Peng J et al., 2014).

The white mold fungus Sclerotinia sclerotiorum is known to attack more than 400 host species and is considered one of the most prolific plant pathogens. The majority of the affected crop species are dicotyledonous, along with a number of agriculturally significant monocotyledonous plants. Some important crops affected by S. sclerotiorum include legumes (soybean), most vegetables, stone fruits and tobacco.

The ascomycete Verticillium dahliae is a soil-borne fungal plant pathogen that causes vascular wilt diseases in a broad range of dicotyledonous host species. V. dahliae can cause severe yield and quality losses in cotton and other important crops such as vegetables, fibers, fruit, nut trees, forest trees and ornamental plants.

The ascomycete fungus Mycospharella gramincola (anamorph: Septoria tritici) is one of the most important foliar diseases of wheat leaves, occurring wherever wheat is grown. Yield losses attributed to this disease range from 25%-50%, and are especially high in Europe, the Mediterranean region and East Africa. Infection by M. gramincola is initiated by air borne ascopores produced on residues of last season's crop. Primary infection usually occurs after seedlings emerge in spring or fall. The mature disease is characterized by necrotic lesions on the leaves and stems of infected plants.

The basidiomycete fungus Sphacelotheca reliana infects corn (Zea mays) systemically, causing Head Smut. Yield loss attributed to the disease is variable, and is directly dependent on the incidence of the disease. The fungus overwinters as diploid teliospores in crop debris or soil. Floral structures are converted to sori containing masses of powdery teliospores that resemble mature galls of common smut.

Examples of crops to be treated and plant diseases (pathogens) to be controlled using the presently disclosed compounds and compositions include, without limitation:

Sugar beet: brown spot disease (Cercospora beticola), black root disease (Aphanomyces cochlioides), root rot disease (Thanatephorus cucumeris), leaf rot disease (Thanatephorus cucumeris), and the like.

Peanut: brown spot disease (Mycosphaerella arachidis), leaf mold (Ascochyta sp.), rust disease (Puccinia arachidis), damping-off disease (Pythium debaryanum), rust spot disease (Alternaria alternata), stem rot disease (Sclerotium rolfsii), black rust disease (Mycosphaerella berkeleyi), and the like.

Cucumber: powdery mildew (Sphaerotheca fuliginea), downy mildew (Pseudoperonospora cubensis), gummy stem blight (Mycosphaerella melonis), wilt disease (Fusarium oxysporum), sclerotinia rot (Sclerotinia sclerotiorum), gray mold (Botrytis cinerea), anthracnose (Colletotrichum orbiculare), scab (Cladosporium cucumerinum), brown spot disease (Corynespora cassiicola), damping-off disease (Pythium debaryanum, Rhizoctonia solani Kuhn), Phomopsis root rot disease (Phomopsis sp.), Bacterial spot (Pseudomonas syringae pv. Lechrymans), and the like.

Tomato: gray mold disease (Botrytis cinerea), leaf mold disease (Cladosporium flavum), late blight disease (Phytophthora infestans), Verticillium wilt disease (Verticillium albo-atrum, Verticillium dahliae), powdery mildew disease (Oidium neolycopersici), early blight disease (Alternaria solani), leaf mold disease (Pseudocercospora fuligena), and the like.

Eggplant: gray mold disease (Botrytis cinerea), black rot disease (Corynespora melongenae), powdery mildew disease (Erysiphe cichoracearum), leaf mold disease (Mycovellosiella nattrassii), sclerotinia rot disease (Sclerotinia sclerotiorum), Verticillium wilt disease (Verticillium dahlia), Mycosphaerella blight (Phomopsis vexans), and the like.

Strawberry: gray mold disease (Botrytis cinerea), powdery mildew disease (Sphaerotheca humuli), anthracnose disease (Colletotrichum acutatum, Colletotrichum fragariae), phytophthora rot disease (Phytophthora cactorum), soft rot disease (Rhizopus stolonifer), fusarium wilt disease (Fusarium oxysporum), verticillium wilt disease (Verticillium dahlia), and the like.

Onion: neck rot disease (Botrytis allii), gray mold disease (Botrytis cinerea), leaf blight disease (Botrytis squamosa), downy mildew disease (Peronospora destructor), Phytophthora porn disease (Phytophthora porn), and the like.

Cabbage: clubroot disease (Plasmodiophora brassicae), soft rot disease (Erwinia carotovora), black rot disease (Xanthomonas campesrtis pv. campestris), bacterial black spot disease (Pseudomonas syringae pv. Maculicola, P.s. pv. alisalensis), downy mildew disease (Peronospora parasitica), sclerotinia rot disease (Sclerotinia sclerotiorum), black spot disease (Alternaria brassicicola), gray mold disease (Botrytis cinerea), and the like.

Common bean: sclerotinia rot disease (Sclerotinia sclerotiorum), gray mold disease (Botrytis cinerea), anthracnose (Colletotrichum lindemuthianum), angular spot disease (Phaeoisariopsis griseola), and the like.

Apple: powdery mildew disease (Podosphaera leucotricha), scab disease (Venturia inaequalis), Monilinia disease (Monilinia mali), black spot disease (Mycosphaerella pomi), valla canker disease (Valsa mali), alternaria blotch disease (Alternaria mali), rust disease (Gymnosporangium yamadae), ring rot disease (Botryosphaeria berengeriana), anthracnose disease (Glomerella cingulata, Colletotrichum acutatum), leaf rot disease (Diplocarpon mali), fly speck disease (Zygophiala jamaicensis), Sooty blotch (Gloeodes pomigena), violet root rot disease (Helicobasidium mompa), gray mold disease (Botrytis cinerea), and the like.

Japanese apricot: scab disease (Cladosporium carpophilum), gray mold disease (Botrytis cinerea), brown rot disease (Monilinia mumecola), and the like.

Persimmon: powdery mildew disease (Phyllactinia kakicola), anthracnose disease (Gloeosporium kaki), angular leaf spot (Cercospora kaki), and the like.

Peach: brown rot disease (Monilinia fructicola), scab disease (Cladosporium carpophilum), phomopsis rot disease (Phomopsis sp.), bacterial shot hole disease (Xanthomonas campestris pv. pruni), and the like.

Almond: brown rot disease (Monilinia taxa), spot blotch disease (Stigmina carpophila), scab disease (Cladosporium carpophilum), red leaf spot disease (Polystigma rubrum), alternaria blotch disease (Alternaria alternata), anthracnose (Colletotrichum gloeospoides), and the like.

Yellow peach: brown rot disease (Monilinia fructicola), anthracnose disease (Colletotrichum acutatum), black spot disease (Alternaria sp.), Monilinia kusanoi disease (Monilinia kusanoi), and the like.

Grape: gray mold disease (Botrytis cinerea), powdery mildew disease (Uncinula necator), ripe rot disease (Glomerella cingulata, Colletotrichum acutatum), downy mildew disease (Plasmopara viticola), anthracnose disease (Elsinoe ampelina), brown spot disease (Pseudocercospora vitis), black rot disease (Guignardia bidwellii), white rot disease (Coniella castaneicola), rust disease (Phakopsora ampelopsidis), and the like.

Pear: scab disease (Venturia nashicola), rust disease (Gymnosporangium asiaticum), black spot disease (Alternaria kikuchiana), ring rot disease (Botryosphaeria berengeriana), powdery mildew disease (Phyllactinia mali), Cytospora canker disease (Phomopsis fukushii), brown spot blotch disease (Stemphylium vesicarium), anthracnose disease (Glomerella cingulata), and the like.

Tea: ring spot disease (Pestalotiopsis longiseta, P. theae), anthracnose disease (Colletotrichum theae-sinensis), Net blister blight (Exobasidium reticulatum), and the like.

Citrus fruits: scab disease (Elsinoe fawcettii), blue mold disease (Penicillium italicum), common green mold disease (Penicillium digitatum), gray mold disease (Botrytis cinerea), melanose disease (Diaporthe citri), canker disease (Xanthomonas campestris pv. Citri), powdery mildew disease (Oidium sp.), and the like.

Wheat: powdery mildew (Blumeria graminis f sp. tritici), red mold disease (Gibberella zeae), brown rust disease (Puccinia recondita), brown snow mold disease (Pythium iwayamai), pink snow mold disease (Monographella nivalis), eye spot disease (Pseudocercosporella herpotrichoides), leaf scorch disease (Septoria tritici), glume blotch disease (Leptosphaeria nodorum), typhula snow blight disease (Typhula incarnata), sclerotinia snow blight disease (Myriosclerotinia borealis), damping-off disease (Gaeumannomyces graminis), ergot disease (Claviceps purpurea), stinking smut disease (Tilletia caries), loose smut disease (Ustilago nuda), and the like.

Barley: leaf spot disease (Pyrenophora graminea), net blotch disease (Pyrenophora teres), leaf blotch disease (Rhynchosporium secalis), loose smut disease (Ustilago tritici, U. nuda), and the like.

Rice: blast disease (Pyricularia oryzae), sheath blight disease (Rhizoctonia solani), bakanae disease (Gibberella fujikuroi), brown spot disease (Cochliobolus miyabeanus), damping-off disease (Pythium graminicola), bacterial leaf blight (Xanthomonas oryzae), bacterial seedling blight disease (Burkholderia plantarii), brown stripe disease (Acidovorax avenae), bacterial grain rot disease (Burkholderia glumae), Cercospora leaf spot disease (Cercospora oryzae), false smut disease (Ustilaginoidea virens), rice brown spot disease (Alternaria alternata, Curvularia intermedia), kernel discoloration of rice (Alternaria padwickii), pink coloring of rice grains (Epicoccum purpurascens), and the like.

Tobacco: sclerotinia rot disease (Sclerotinia sclerotiorum), powdery mildew disease (Erysiphe cichoracearum), phytophthora rot disease (Phytophthora nicotianae), and the like.

Tulip: gray mold disease (Botrytis cinerea), and the like.

Sunflower: downy mildew disease (Plasmopara halstedii), sclerotinia rot disease (Sclerotinia sclerotiorum), and the like.

Bent grass: Sclerotinia snow blight (Sclerotinia borealis), Large patch (Rhizoctonia solani), Brown patch (Rhizoctonia solani), Dollar spot (Sclerotinia homoeocarpa), blast disease (Pyricularia sp.), Pythium red blight disease (Pythium aphanidermatum), anthracnose disease (Colletotrichum graminicola), and the like.

Orchard grass: powdery mildew disease (Erysiphe graminis), and the like.

Soybean: purple stain disease (Cercospora kikuchii), downy mildew disease (Peronospora manshurica), phytophthora rot disease (Phytophthora sojae), rust disease (Phakopsora pachyrhizi), sclerotinia rot disease (Sclerotinia sclerotiorum), anthracnose disease (Colletotrichum truncatum), gray mold disease (Botrytis cinerea), Sphaceloma scab (Elsinoe glycines), melanoses (Diaporthe phaseolorum var. sojae), and the like.

Potato: hytophthora rot disease (Phytophthora infestans), early blight disease (Alternaria solani), scurf disease (Thanatephorus cucumeris), verticillium wilt disease (Verticillium albo-atrum, V. dahlia, V. nigrescens, and the like.

Banana: Panama disease (Fusarium oxysporum), Sigatoka disease (Mycosphaerella fijiensis, M. musicola), and the like.

Rapeseed: sclerotinia rot disease (Sclerotinia sclerotiorum), root rot disease (Phoma lingam), black leaf spot disease (Alternaria brassicae), and the like.

Coffee: rust disease (Hemileia vastatrix), anthracnose (Colletotrichum coffeanum), leaf spot disease (Cercospora coffeicola), and the like.

Sugarcane: brown rust disease (Puccinia melanocephala), and the like.

Corn: zonate spot disease (Gloeocercospora sorghi), rust disease (Puccinia sorghi), southern rust disease (Puccinia polysora), smut disease (Ustilago maydis), brown spot disease (Cochliobolus heterostrophus), northern leaf blight (Setosphaeria turcica), and the like.

Cotton: seedling blight disease (Pythium sp.), rust disease (Phakopsora gossypii), sour rot disease (Mycosphaerella areola), anthracnose (Glomerella gossypii), and the like.

### V. Pesticides

The presently disclosed compounds, including compounds according to Formulas (I), (Ia), (Ib), (II), (IIa), (III), (IIIa), (IV), (V), (VI), (VII) and (VIII), are useful for enhancing the effect of a variety of agrochemicals, including fungicides, antiviral agents, bactericides, herbicides, insecticidal/acaricidal agents, molluscicides, nematicides, soil pesticides, plant control agents, synergistic agents, fertilizers and soil conditioners.

In one embodiment, the presently disclosed compounds are useful for enhancing the fungicidal effect of a variety of fungicides. Fungicides for use in combination with the enhancers disclosed herein are well known to those of skill in the art and include, without limitation those set forth by class in Table 2:

TABLE 2

| Family & Group # | Common Names | Trade Names (Combination Products) |
|---|---|---|
| Benzimidazole (Group 1) | benomyl | Benlate, Tersan 1991 |
| | thiabendazole | Arbotect 20-S, Decco Salt No. 19, LSP |
| | | Flowable Fungicide, Mertect 340-F |
| | thiophanate-methyl | Cavalier, Cleary's 3336, OHP 6672, Regal |
| | | SysTec, Tee-Off, |
| | | T-Methyl 4.5F AG, TM 85, Topsin M |
| Dicarboximide (Group 2) | iprodione | Epic 30, Ipro, Meteor, Nevado, OHP |
| | | Chipco 26019, Rovral, (Interface) |
| | vinclozolin | Curalan, Ronilan |
| Phenylpyrroles (Group 12) | fludioxonil | Cannonball, Emblem, Maxim, Medallion, |
| | | Mozart, Scholar, Spirato, (Academy, |
| | | Miravis Prime, Palladium, Switch) |
| Anilinopyrimidines (Group | cyprodinil | Vangard (Palladium, Switch, Inspire |
| 9) | | Super) |
| | pyrimethanil | Penbotec, Scala, (Luna Tranquility) |
| Hydroxyanilide (Group 17) | fenhexamid | Decree, Elevate, Judge |
| | fenpyrazamine | Protexio |
| Carboxamide (Group 7) | boscalid | Emerald, Endura, (Encartis, Honor, |
| | | Pageant, Pristine) |
| | carboxin | Vitavax |
| | fluopyram | Luna Privilege, Velum Prime (Broadform, |
| | | Luna Experience, Luna Sensation, Luna |
| | | Tranquility, Propulse) |
| | flutolanil | Contrast, Moncut, ProStar |
| | fluxapyroxad | (Lexicon, Merivon, Orkestra) |
| | inpyrfluxam | Excalia |
| | isofetamid | Kenja |
| | oxycarboxin | Carboject, Plantvax |
| | penthiopyrad | Fontelis, Velista, Vertisan |
| | pydiflumetofen | Miravis, Posterity, Miravis Ace A (Miravis |
| | | Neo, Miravis Prime, Miravis Duo, Miravis |
| | | Top) |
| | solatenol | Aprovia (Contend A, Elatus, Mural) |
| | (benzovindiflupyr) | |
| Phenylamide (Group 4) | mefenoxam | Apron, Ridomil Gold, Subdue MAXX, |
| | | (Quadris Ridomil Gold, Uniform) |
| | metalaxyl | Acquire, Allegiance, MetaStar, Ridomil, |
| | | Sebring, Subdue |
| | oxadixyl | Anchor |
| Phosphonate (Group P7) | aluminum tris | Aliette, Flanker, Legion, Signature, Areca |
| | Phosphorous Acid | Agri-Fos, Alude, Appear, Fiata, Fosphite, |
| | | Phospho Jet, Phostrol, Rampart, Reload |
| Cinnamic acid (Group 40) | dimethomorph | Forum, Stature, (Orvego, Zampro) |
| | mandipropamid | Micora, Revus, (Revus Top) |
| OSBPI (Group 49) | oxathiapiprolin | Segovis |
| Triazoles carboxamide | ethaboxam | V-10208 |
| (Group 22) | | |
| Group 27 | cymoxanil | Curzate, (Tanos) |
| Carbamate (Group 28) | propamocarb | Banol, Previcur, Proplant, Tattoo |
| Benzamide (Group 43) | fluopicolide | Adorn, Presidio |

Demethylation-inhibiting (Group 3)

| | | |
|---|---|---|
| Piperazines | triforine | Funginex, Triforine |
| Pyrimidines | fenarimol | Focus, Rubigan, Vintage |
| Imidazole | imazalil | Fungaflor, (Raxil MD Extra) |
| | triflumizole | Procure, Terraguard, Trionic |
| Triazoles | cyproconizole | Sentinel |
| | difenoconazole | Dividend, Inspire, (Academy, Briskway, |
| | | Contend A, Inspire Super, Quadris Top, |
| | | Revus Top) Miravis Duo |
| | fenbuconazole | Enable, Indar |
| | flutriafol | Topguard, (Topguard EQ) |
| | mefentrifluconazole | Maxtima (Navicon) |
| | metconazole | Quash, Tourney |
| | ipconazole | Rancona |
| | myclobutanil | Eagle, Hoist, Immunox, Laredo, Nova, |
| | | Rally, Sonoma, Systhane |
| | propiconazole | Alamo, Banner, Break, Bumper, Infuse, |
| | | Kestrel Mex, Miravis Ace B, PropiMax, |
| | | ProPensity, Strider, Tilt, Topaz, (Aframe |
| | | Plus, Concert, Contend B, Headway, Quilt |
| | | Xcel, Stratego) |
| | prothioconazole | Proline (Propulse) |
| | tebuconazole | Bayer Advanced, Elite, Folicur, Lynx, |
| | | Mirage, Orius, Raxil, Sativa, Tebucon, |
| | | Tebuject, Tebusha, Tebustar, Toledo, |
| | | (Absolute, Luna Experience, |
| | | Unicorn), etc. |
| | tetraconazole | Mettle |
| | triadimefon | Bayleton, Strike, (Armada, Tartan, Triigo) |
| | triadimenol | Baytan |
| | triticonazole | Charter, Trinity, (Pillar) |
| Morpholine (Group 5) | piperalin | Pipron |
| | spiroxamine | Accrue |
| Group U6 | cyflufenamid | Torino |
| Group 50 | metrafenone | Vivando |
| | pyriofenone | Prolivo |
| QoI Strobilurins (Group 11) | azoxystrobin | Abound, Aframe, Dynasty, Heritage, |
| | | Protété, Quadris, Quilt, (Aframe Plus, |
| | | Briskway, Contend B, Dexter Max, Elatus, |
| | | Headway, Mural, Quadris Top, |
| | | Quilt Xcel, Renown, Topguard EQ, |
| | | Uniform) |
| | famoxadone | (Tanos) |
| | fenamidone | Fenstop, Reason |
| | fluoxastrobin | Aftershock, Disarm, Evito, Fame |
| | kresoxim-methyl | Cygnus, Sovran |
| | mandestrobin | Intuity, Pinpoint |
| | picoxystrobin | Aproach |
| | pyraclostrobin | Cabrio, Empress, Headline, Insignia, |
| | | Stamina, (Honor, Lexicon, Merivon, |
| | | Navicon, Orkestra, Pageant, Pillar, |
| | | Pristine) |
| | trifloxystrobin | Compass, Flint, Gem, (Absolute, Armada, |
| | | Broadform, Interface, Luna Sensation, |
| | | Stratego, Tartan, Trigo) |
| Quinoline (Group 13) | quinoxyfen | Quintec |

Inorganic Compounds

| | | |
|---|---|---|
| Coppers (Group M1) | bordeaux | None |
| | copper ammonium | Copper Count-N |
| | complex | |
| | copper hydroxide | Champ, Champion, Kalmor, Kentan, |
| | | Kocide, Nu-Cop |
| | copper oxide | Nordox |
| | copper oxychloride | C-O-C-S, Oxycop |
| | copper sulfate | Cuprofix Disperss, many others |
| Sulfur (Group M2) | sulfur | Cosavet, Kumulus, Microthiol |
| | | Disperss, Thiosperse |
| Lime sulfur | Ca polysulfides | Lime Sulfur, Sulforix |
| Ethylenebisdithiocarbamates | mancozeb | Dithane, Fore, Penncozeb, Protect, Manex, |
| (EBDC) (Group M3) | | Manzate, Roper, Wingman, (Dexter Max, |
| | | Gavel) |
| | maneb | Maneb |
| | metiram | Polyram |
| EBDC-like (Group M3) | ferbam | Carbamate, Ferbam |
| | thiram | Difiant, Spotrete, Thiram |
| | ziram | Ziram |
| Aromatic Hydrocarbon | dicloran (DCNA) | Allisan, Botran |
| (Group 14) | etridizole | Terrazole, Truban |
| | pentachloronitrobenzene | Autilus, Defend, Engage, PCNB, |
| | | Terraclor, (Premion) |
| Chloronitrile (Group M5) | chlorothalonil | Bravo, Daconil, Docket, Echo, Ensign, |
| | | Exotherm Termil, Funginil, Legend, |
| | | Manicure, Pegasus, Terranil, (Concert, |
| | | Spectro) |
| Phthalimides (Group M4) | captan | Captan |
| Guanidines (Group U12) | dodine | Syllit |
| Qil fungicides (Group 21) | cyazofamid | Ranman, Segway |
| Polyoxin (Group 19) | polyoxin | Affirm, Endorse, Oso, Ph-D, Tavano, |
| | | Veranda |
| Group 29 | fluazinam | Omega, Secure |
| Thiazolidine (U13) | flutianil | Gatten |

Fungicides are cataloged more broadly by the Fungicide Resistance Action Committee (FRAC) in the FRAC Code List 2022 and reproduced in Appendix 1 and which is incorporated herein by reference in its entirety.

In one embodiment, a presently disclosed enhancer compound is used in combination with one or more compound from the Families or Groups set forth in Table 2, Appendix 1, or both. In certain embodiments, a presently disclosed enhancer is used in combination with one or more fungicides recited in column 1 of Table 2. By way of example, such use of an enhancer compound of Formulas (I), (Ia), (Ib), (II), (IIa), (III), (IIIa), (IV), (V), (VI), (VII) and/or (VIII), in combination with a fungicide can include administration at the same or different times. In one embodiment an enhancer compound is administered prior to a fungicide. In one embodiment, an enhancer compound is administered after a fungicide.

In particular embodiments, a disclosed enhancer is used in combination with one or more of a fungicide selected from the benzimidazoles, dicarboximides, phenylpyrroles, anilinopyrimidines, hydroxyanilides, carboxamides, phenyl amides, phosphonates, cinnamic acids, oxysterol binding protein inhibitors (OSBPI), triazole carboxamides, cymoxanil, carbamates, benzamides, demethylation inhibiting piperazines, demethylation inhibiting pyrimidines, demethylation inhibiting azoles, including imidazoles and triazoles, such as cyproconazole, difenoconazole, fenbuconazole, flutriafol, mefentrifluconazole, metconazole, ipconazole, prothioconazole, tebuconazole, tetraconazole, triadimefon, triadimenol, triticonazole, morpholines, cyflufenamid, metrafenone, pyriofenone, strobilurins, copper ammonium complex, copper hydroxide, copper oxide, copper oxychloride, copper sulfate, sulfur, lime sulfur, ethylenebisdithiocarbamates, aromatic hydrocarbons, phthalimides, guanidines, polyoxins, fluazinam and thiazolidines.

Particular fungicides that are potentiated by use in combination with an enhancer according to the methods herein by administration of an apyrase inhibitor are coppers, such as copper octanoate, copper hydroxide and the like, myclobutanil, propiconazole, tebuconazole, epoxiconazole, difenoconazole, triticonazole, and prothioconazole.

In one embodiment, the combined treatment with a selected fungicide and an enhancer according to the present disclosure provides synergistic fungicidal activity against plant pathogenic fungi.

In one embodiment, the disclosure provides compositions and methods of treating plants or plant seeds infected with or at risk of being infected with a fungal pathogen. In one embodiment compositions of the present disclosure comprise a formulation of a fungicide, an enhancer and a phytologically acceptable carrier. In another embodiment, the fungicide and enhancer are administered in separate compositions. In further embodiments, an agricultural or horticultural fungicide is used in combination with other compounds in addition to the presently disclosed apyrase inhibitors. As with the apyrase inhibitors, such other compounds can be administered in the same or separate compositions as the fungicide. Examples of the other components include known carriers to be used to conduct formulation. Additional examples thereof include conventionally-known herbicides, insecticidal/acaricidal agents, nematocides, soil pesticides, plant control agents, synergistic agents, fertilizers, soil conditioners, and animal feeds. In one embodiment, the inclusion of such other components yields synergistic effects on crop growth.

In one embodiment, the presently disclosed compounds, including compounds according to Formulas (I), (Ia), (Ib), (II), (IIa), (III), (IIIa), (IV), (V), (VI), (VII) and (VIII), are used to potentiate the effect of a herbicide. Exemplary herbicides for use in combination with the present compounds are known to those of skill in the art and include, without limitation, those described in Appendix 2. By way of example, suitable herbicides for use in combination with the present compounds include inhibitors of acetyl CoA synthase, inhibitors of acetolactate synthesis, inhibitors of microtubule assembly, inhibitors of microtubule organization, auxin mimics, photosynthesis inhibitors, deoxy-D-xylulose phosphate synthase inhibitors, enolpyruvyl shikimate phosphate synthase inhibitors, phytoene desaturase inhibitors, glutamine synthetase inhibitors, dihydropteroate synthesis inhibitors, protoporphyrinogen oxidase inhibitors, cellulose synthesis inhibitors, uncouplers, hydroxyphenyl pyruvate dioxygenase inhibitors, fatty acid thioesterase inhibitors, serine-threonine protein phosphatase inhibitors, solanesyl diphosphate synthase inhibitors, inhibitors of very long-chain fatty acid synthesis, homogentisate solanesyltransferase inhibitors, lycopene cyclase inhibitors,

In one embodiment, the presently disclosed compounds, including compounds according to Formulas (I), (Ia), (Ib), (II), (IIa), (III), (IIIa), (IV), (V), (VI), (VII) and (VIII), are used to potentiate the effect of an insecticide. Exemplary insecticides for use in combination with the present compounds are known to those of skill in the art and include, without limitation, those described in Appendix 3.

### VI. Formulations

The present disclosure provides specific apyrase inhibitors, including compounds of Formulas (I), (Ia), (Ib), (II), (IIa), (III), (IIIa), (IV), (V), (VI), (VII) and (VIII), to enhance the potency of pesticides to effectively restrict the growth of plant pathogenic species. In certain non-limiting embodiments, the apyrase inhibitors can be provided at: from about 0.01 to about 80% weight to weight in a final composition, or from about 25% to about 55%, such as from about 30% to about 50%, from about 35% to about 45%, such as about 0.01, 0.05, 0.1, 0.5, 1.0, 1.1, 1.2, 1.3, 1.4, 1.5, 1.6, 1.7, 1.8, 1.9, 2.0, 2.5, 3.0, 4.0, 5.0, 7.5, 10, 20, 30, 40, 50, 55, 60 or 80% weight to weight in a final composition. In one embodiment the apyrase inhibitors are provided in liquid form at from about 0.01 to about 50%, such as from about 15% to about 50%, from about 20% to about 45%, from about 25% to about 40%, such as about 0.01, 0.05, 0.1, 0.5, 1.0, 1.1, 1.2, 1.3, 1.4, 1.5, 1.6, 1.7, 1.8, 1.9, 2.0, 2.5, 3.0, 4.0, 5.0, 7.5, 10, 15, 20, 30, 40 or 50% volume to volume in a final diluted composition. The skilled artisan will recognize that the formulation of the pesticide, the apyrase inhibitor or a combination thereof can be provided in a concentrate that can be diluted prior to use, or can be provided in a diluted form ready for treatment.

The enhancer, pesticide and combinations thereof are not particularly limited by the dosage form. Examples of the dosage form include wettable powders, emulsions, emulsifiable concentrates, oil-dispersible liquids, powders, granules, water-soluble agents, suspensions, granular wettable powders, and tablets. The method for preparing formulation is not particularly limited, and conventionally-known methods may be adopted depending on the dosage form.

Several formulation examples are described below. The preparation formulations shown below are merely examples, and may be modified within a range not contrary to the essence of the present disclosure. For example, additional active and inert components may be added to the formulations below.

“Part” means “part by mass” unless otherwise specified.

### Formulation Example 1: Wettable Powders

40 parts of an enhancer disclosed herein, 53 parts of diatomaceous earth, 4 parts of ethoxylated higher alcohol sulfate ester combined with a suitable solid carrier such as magnesium sulfate, and 3 parts of alkyl naphthalene sulfonate are mixed uniformly, and then finely pulverized to obtain wettable powders containing 40 parts by mass of the enhancer.

### Formulation Example 2: Emulsifiable Concentrates

3 parts of an enhancer disclosed herein, 60 parts of mixed petroleum distillates, 27 parts of dimethyl lactamide, and 10 parts of tristyrylphenol ethoxylates are mixed and dissolved to obtain an emulsifiable concentrate containing 3% by mass of the enhancer.

### Formulation Example 3: Granules

5 parts of an enhancer disclosed herein, 10 parts of talc, 38 parts of clay, 10 parts of bentonite, 30 parts of sodium lignosulfonate and 7 parts of sodium alkyl sulfate are mixed uniformly, and then finely pulverized, followed by conducting fluidized bed granulation to make the median particle diameter thereof be 0.2 to 2.0 mm, and thus granules containing 5% by mass of an enhancer on a dry weight basis disclosed herein are obtained.

### Formulation Example 4: Granules

5 parts of an enhancer disclosed herein, 73 parts of clay, 20 parts of bentonite, 1 part of sodium dioctyl sulfosuccinate, and 1 part of potassium phosphate are mixed and then pulverized, followed by adding water thereto, and then kneading the mixture. Then, extrusion granulation is conducted, and the resultant is dried to obtain granules containing 5% by mass of the enhancer on a dry weight basis.

### Formulation Example 5: Suspensions

10 parts of an enhancer disclosed herein, 4 parts of polyoxyethylene alkyl ether, 2 parts of 3 kDa sodium polycarboxylate as dispersant, 10 parts of glycerin, 0.2 parts of xanthan gum, 0.1 parts of biocides as stabilizer, 0.1 parts of organosilicone antifoam emulsion and 73.6 parts of water are mixed, and then wet pulverized until the particle size is 3 microns or less to obtain a suspension containing 10% by mass of the enhancer.

### Formulation Example 6: Oil Dispersible Concentrates

40 parts of an enhancer disclosed herein, 5 parts of Atlox 4914, 5 parts of organo-modified bentonite and 50 parts of methylated rapeseed oil as carrier are mixed uniformly and then wet pulverized until the median particle size is 3 microns or less to obtain an oil dispersible concentrate containing 40% by mass of the enhancer.

The skilled artisan will recognize that the various compositions are used commercially at varying concentrations and formulations. For example, it is common for fungicides to be formulated as liquids commercially at 10-40% concentrations. In one embodiment, the presently disclosed enhancers allow the use of a lower amount of a given fungicide due to the enhanced efficacy of fungicide in combination with an enhancer disclosed herein.

### VII. Methods for Assessing Enhancer Activity

The presently disclosed compounds exhibit activity against a variety of pathogens. Their activity is assessed in part according to the following assays:

### Method 1: In Vitro Apyrase Assay:

Apyrase inhibitors useful as enhancers of pesticidal activity are assessed using an in vitro assay. The method of Windsor, Bio Techniques 33:1024-1030 (November 2002) was used as follows:

### Screen for Apyrase Inhibitors —

96 well plates were used for the assay: (Greiner bio-one: REF-655901-96 well, PS, F-bottom, Clear, Non-binding)

### Buffers:

Reaction Buffer: 60 mM Hepes; 3 mM MgCl₂, 3 mM CaCl₂ and 3 mM ATP (pH 6.5)
Development Buffer A: 2% aqueous ammonium molybdate
Development Buffer B: 11% ascorbic acid in 37.5% TCA in water
Stop buffer C: 2% trisodium citrate in 2% acetic acid solution in water
   Add 100 μl of reaction buffer to each well.
   Add 100 of DMSO (control) or inhibitor/compound or compounds such as N1915 or orthovanadate to each well. (use inhibitor conc at 1 mM; orthovanadate at 2 mM and N1915 at 1 mM)
   Add 100 of apyrase (concentration based on optimization—Dilute 1 U/μl enzyme to different concentrations such as 0.1 U, 0.05 U, 0.0025, 0.001 U, 0.0005 U- to find a good range)
   Incubate plate at room temperature for 1 hr
   Mix development buffer A and B in the ratio of 1:1.5 (just before use).
   Add 50 ul of A:B mix in each well (incubate for 2 mins)
   Add 50 ul of C in each well
   Measure/Read Absorbance of plate @630 nm
      Inhibitory data for the apyrase assay described above are provided for selected compounds in Table 3:

TABLE 3

| ID | % Inhibition of Apyrase |
|---|---|
| I-1 | 74% |
| I-2 | 60% |
| I-3 | 46% |
| I-4 | 24% |
| I-5 | 22% |
| I-6 | 22% |
| I-7 | 20% |
| I-8 | 19% |
| I-9 | 19% |
| I-10 | 17% |
| I-11 | 17% |
| I-12 | 16% |
| I-13 | 16% |
| I-14 | 16% |
| I-15 | 15% |
| I-16 | 14% |
| I-17 | 14% |
| I-18 | 13% |
| I-19 | 13% |
| I-20 | 12% |
| I-21 | 12% |
| I-22 | 12% |
| I-23 | 12% |
| I-24 | 11% |
| I-25 | 11% |
| I-26 | 11% |
| I-27 | 10% |
| I-28 | 10% |
| I-29 | 10% |
| I-30 | 10% |
| I-31 | 10% |
| I-32 | 10% |
| I-33 | 10% |
| I-34 | |
| I-35 | |
| I-36 | |
| I-37 | |
| I-38 | |
| I-39 | |
| I-40 | |
| I-41 | |
| I-43 | |
| I-44 | |
| I-45 | |
| I-46 | |
| I-47 | |
| I-48 | |
| I-49 | |
| I-50 | |
| I-51 | |
| I-52 | |
| I-53 | |
| I-54 | |
| I-55 | |
| I-56 | |
| I-57 | |
| I-58 | |
| I-59 | |
| I-60 | |
| I-61 | |
| I-62 | |
| I-63 | |
| I-64 | |
| I-65 | |
| I-66 | |
| I-67 | |
| I-68 | |
| I-69 | |
| I-70 | |
| I-71 | |
| I-72 | |
| I-73 | |
| I-74 | |
| I-75 | |
| I-76 | |
| I-77 | |
| I-78 | |
| I-79 | |
| I-80 | |
| I-81 | |
| I-83 | |
| I-84 | |
| I-85 | |
| I-86 | |
| I-87 | |
| I-88 | |
| I-89 | |
| I-90 | |
| I-91 | |
| I-92 | |
| I-93 | |
| I-94 | |
| I-95 | |
| I-96 | |
| I-97 | |
| I-98 | |
| I-99 | |
| I-100 | |
| I-101 | |
| I-102 | |
| I-103 | |
| I-104 | |
| I-105 | |
| I-106 | |
| I-107 | |
| I-108 | |
| I-109 | |
| I-110 | |
| I-111 | |
| I-112 | |
| I-113 | |
| I-114 | |
| I-115 | |
| I-116 | |
| I-117 | |
| I-118 | |
| I-119 | |
| I-120 | |
| I-121 | |
| I-122 | |
| I-123 | |
| I-124 | |
| I-125 | |
| I-126 | |
| I-127 | |
| I-128 | |
| I-129 | |
| I-130 | |
| I-131 | |
| I-132 | |
| I-133 | |
| I-134 | |
| I-135 | |
| I-136 | |
| I-137 | |
| I-138 | |
| I-139 | |
| I-140 | |
| I-141 | |
| I-142 | |
| I-143 | |
| I-144 | |
| I-145 | |
| I-146 | |
| I-147 | |
| I-148 | |
| I-149 | |
| I-150 | |
| I-151 | |
| I-152 | |
| I-153 | |
| I-154 | |
| I-155 | |
| I-156 | |
| I-157 | |
| I-158 | |
| I-159 | |
| I-160 | |
| I-161 | |
| I-162 | |
| I-164 | |
| I-165 | |
| I-166 | |
| I-167 | |
| I-168 | |
| I-169 | |
| I-170 | |
| I-171 | |
| I-172 | |
| I-174 | |
| I-175 | |
| I-176 | |
| I-177 | |
| I-178 | |
| I-179 | |
| I-180 | |
| I-181 | |
| I-182 | |
| I-183 | |
| I-184 | |
| I-185 | |
| I-186 | |
| I-187 | |
| I-188 | |
| I-189 | |
| I-190 | |
| I-191 | |
| I-192 | |
| I-193 | |
| I-194 | |
| I-195 | |
| I-196 | |
| I-197 | |
| I-198 | |
| I-199 | |
| I-200 | |
| I-201 | |
| I-202 | |
| I-203 | |
| I-204 | 74 |
| I-205 | 68 |
| I-206 | 68 |
| I-207 | 68 |
| I-208 | 66 |
| I-209 | 65 |
| I-210 | 63 |
| I-211 | 56 |
| I-212 | 56 |
| I-213 | 55 |
| I-214 | 55 |
| I-215 | 46 |
| I-216 | 44 |
| I-217 | 44 |
| I-218 | 42 |
| I-219 | 40 |
| I-220 | 25 |
| I-221 | 23 |
| I-222 | 12 |
| I-223 | 10 |
| I-224 | 10 |
| I-225 | 6 |
| I-226 | 1 |
| I-227 | |
| I-228 | |
| I-229 | |
| I-230 | |
| I-231 | |
| I-232 | |
| I-233 | |
| I-234 | |
| I-235 | |

With reference to Table 3, percent inhibition of apyrase is reported as the rounded average of two assay results. A blank cell indicates either <10% observed inhibition or a large difference between repetitions. In certain examples, the lack of observed inhibition is due to a lack of solubility of the compound under assay conditions, rather than a lack of apyrase inhibitory activity. Roux compound 15 inhibited apyrase in this assay at about 60%.

### Method 2: In Vitro Assessment of Combination Activity

Selected compounds were assessed in combination with fungicides against a range of commercially important plant pathogenic fungi.

The test was conducted as follows. A fungicide was applied to a fungal plant pathogen at a rate slightly below that at which it gave any control, in combination with a suitable dose of the test compound. The test compound was recorded as active if control of the pathogen was observed.

In more detail, the test was conducted as follows. For each combination of fungicide, pathogen and test compound, the following wells were used. Well 1 contained a fungal pathogen growing on agar, and a fungicide at a rate just below that at which it gave any control of the pathogen. Well 2 was the same as Well 1, except that the test compound was also added at Rate 1. Well 3 was the same as Well 2, except that the test compound was added at Rate 2, where Rate 2 was higher than Rate 1. Finally, as a benchmark, Well 4 was the same as Well 1, except that it contained the fungicide at a higher rate, at which it gave partial control of the pathogen. Each of the Wells 1 to 4 were run in duplicate, giving a total of 8 wells for each combination of fungicide, pathogen and test compound. For each well, after a suitable period of incubation, a visual assessment of the % control of the pathogen by the fungicide was made. Test compounds were scored as inactive, active or highly active.

The following fungicides were used in this assay: azoxystrobin, fluxapyroxad, and desthio prothioconazole. The following fungal pathogens were used in this assay. First, a strain of Zymoseptoria tritici with a reduced susceptibility to strobilurin fungicides; second a strain of Zymoseptoria tritici with a reduced susceptibility to SDHI fungicides (i.e., those that inhibit succinate dehydrogenase); and third, Microdochium nivale. In this assay, Roux Compound 15 exhibited no activity. In contrast present compound 1-4, which inhibited only 24% of apyrase activity in Method 1, was highly effective in the combination assay, showing significant activity against all three fungal pathogens in combination with each of the three fungicides.

Compound 1-223, which inhibited only 10% of apyrase activity in Method 1, showed significant activity in combination with fluxapyroxad against Microdochium nivale and in combination with desthio prothioconazole against Zymoseptoria tritici with a reduced susceptibility to strobilurin fungicides.

Compound 1-214, which inhibited 55% in Method 1, showed significant activity in combination with fluxapyroxad against Microdochium nivale, and in combination with desthio prothioconazole against Zymoseptoria tritici with a reduced susceptibility to strobilurin fungicides and Zymoseptoria tritici with a reduced susceptibility to SDHI fungicides.

Compound 1-9, which inhibited 19% in Method 1, showed significant activity in combination with azoxystrobin against Microdochium nivale, and in combination with and in combination with desthio prothioconazole against Zymoseptoria tritici with a reduced susceptibility to SDHI fungicides.

Compound 1-215, which inhibited 46% in Method 1, showed significant activity in combination with fluxapyroxad against Microdochium nivale.

Surprisingly, exemplary compounds, including compounds that showed lesser activity than Roux Compound 15 in the in vitro inhibition assay of Method 1 above, showed significant activity in the combination assay where Roux Compound 15 demonstrated zero activity.

### Method 3: Greenhouse Crop Tests

In this method, exemplary compounds were evaluated for their ability to control Zymoseptoria tritici on wheat, Botrytis cinerea on tomatoes, Asian Soya Rust (Phakopsora pachyrhizi) on soybean, and Brown Rust (Puccinia recondita) on wheat, in a controlled greenhouse environment in combination with one of four fungicides, Amistar, Imtrex, Proline or Balaya. In these studies, soybean cultivar Siverka, tomato (Money maker) and wheat plants (JB Diego) were used. Seeds were sown in 9 cm diameter pots to a depth of 1 to 2 cm using Petersfield potting compost (75% medium grade peat, 12% screened sterilized loam, 3% medium grade vermiculite, 10% grit (5 mm screened, lime free), 1.5 kg PG mix per m3, lime to pH5.5-6.0 and wetting agent (Vitax Ultrawet 200 ml per m3) and germinated/grown at 23° C. under a 16 h day/8 h night light regime. Plants were treated two to three weeks after sowing when they were at the BBCH 11 growth stage (first pair of true leaves (unifoliate) unfolded. A track sprayer was used to treat the plants with the mixture of commercial fungicide and test compound using a water volume of 200 L/ha. Plants were inoculated with the appropriate fungi (pathogen) 24 hours after treatment. Fungal pathogens used were Botrytis cinerea (Grey mold on tomato plants), Zymoseptoria tritici (Septoria leaf blotch on wheat plants), Puccinia triticina (Brown rust on wheat plants) and Phakopsora pachyrhyzi (Asian soy rust on soybean plants). Four replicates were used for each combination of fungicide, pathogen and test compound. Each plant was evaluated once the disease symptoms were fully expressed between seven to twenty days (depending on the pathogen) for % control of the disease. Appropriate controls were used for all experiments, including an ‘inoculation check’ wherein plants were inoculated with their specific pathogen to assess disease levels. Also, each commercial fungicide was tested on its own as a part of each treatment, this being benchmark against which the experimental compounds were evaluated. Exemplary compounds demonstrated enhanced disease control in combination with fungicides as compared to disease control observed with fungicide alone. That is, the present compounds, although not fungicidal by themselves, enhance the activity of fungicides.

In these studies the fungicide was applied at the following rates

| | Amistar | Imtrex | Proline | Balaya |
|---|---|---|---|---|
| Zymoseptoria | 0.1 L/ha | 0.02 L/ha | 0.0075 L/ha | 0.0015 L/ha |
| tritici | | | | |
| Botrytis | 0.03 L/ha | 0.005 L/ha | 0.00075 L/ha | 0.0015 L/ha |
| Asian Soya | 0.02 L/ha | 0.05 L/ha | 0.15 L/ha | 0.03 L/ha |
| Rust | | | | |
| Brown Rust | 0.03 L/ha | 0.35 L/ha | 0.125 L/ha | 0.2 L/ha |

In this method, Amistar or Balaya, in combination with Compound 1-214 applied at 20 ppm gave notably superior control of Zymoseptoria tritici compared to Amistar or Balaya alone or in combination with Roux Compound 15 applied at 30 ppm. Imtrex or Proline, in combination with Compound 1-214 at 20 ppm, gave similar control of Zymoseptoria tritici to Imtrex or Proline in combination with Roux Compound 15 applied at 15 and 30 ppm, and superior control to Imtrex or Proline alone. Imtrex and Balaya, in combination with Compound 1-214 applied at 20 ppm, gave notably superior control of Botrytis than Imtrex or Balaya alone or in combination with Roux Compound 15 applied at 30 ppm. The activities of Imtrex against Brown Rust, and of Amistar, Proline and Balaya against Asian Soybean Rust, were all substantially enhanced by the addition of Compound 1-214 at 20 ppm.

Proline and Balaya, with the addition of Compound 1-223, applied at 20 ppm exhibited notable superior results in controlling Zymoseptoria tritici than Proline or Balaya alone or in combination with Roux Compound 15 applied at 30 ppm.

Amistar, in combination with Compound 1-4, exhibited comparable levels of control of Botrytis to Amistar in combination with Roux Compound 15 at the same rate. In both cases, control was substantially higher than with Amistar alone. By contrast, Amistar, in combination with Compound 1-4 at 15 ppm, gave much higher levels of control of Botrytis than Amistar in combination with Roux Compound 15 at this same lower rate. Again, control was substantially higher than with Amistar alone. Proline, in combination with Compound 1-4 at 30 ppm, gave similar levels of control of Botrytis to Proline in combination with Roux Compound 15 at the same rate. In both cases, the activity was notably higher than with Proline alone. Imtrex, in combination with Compound 1-4 at 30 ppm, gave far superior control of Botrytis than Imtrex alone or in combination with Roux Compound 15 at the same rate. Finally, Balaya, in combination with Compound 1-4 applied at 15 ppm, was significantly more active against Botrytis than Balaya alone or in combination with Roux Compound 15 at the same rate. In fact, Balaya, in combination with Roux Compound 15 at 15 ppm, was no more active against Botrytis than Balaya alone.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

APPENDIX 1

| | | | CHEMICAL OR | | | |
|---|---|---|---|---|---|---|
| | TARGET SITE | GROUP | BIOLOGICAL | COMMON | | FRAC |
| MOA | AND CODE | NAME | GROUP | NAME | COMMENTS | CODE |
| A: | A1 | PA - fungicides | acylalanines | benalaxyl | Resistance and cross | 4 |
| nucleic acids | RNA polymerase I | (PhenylAmides) | | benalaxyl-M | resistance well | |
| metabolism | | | | (=kiralaxyl) | known in various | |
| | | | | furalaxyl | Oomycetes but mechanism | |
| | | | | metalaxyl | unknown. | |
| | | | | metalaxyl-M | High risk. | |
| | | | | (=mefenoxam) | See FRAC Phenylamide | |
| | | | oxazolidinones | oxadixyl | Guidelines for resistance | |
| | | | butyrolactones | ofurace | management | |
| | A2 | hydroxy- | hydroxy- | bupirimate | Medium risk. Resistance and | 8 |
| | adenosin- | (2-amino-) | (2-amino-) | dimethirimol | cross resistance known in | |
| | deaminase | pyrimidines | pyrimidines | ethirimol | powdery mildews. | |
| | | | | | Resistance management | |
| | | | | | required. | |
| | A3 | heteroaromatics | isoxazoles | hymexazole | Resistance not known. | 32 |
| | DNA/RNA synthesis | | isothiazolones | octhilinone | | |
| | (proposed) | | | | | |
| | A4 | carboxylic acids | carboxylic acids | oxolinic acid | Bactericide. | 31 |
| | DNA topoisomerase | | | | Resistance known. | |
| | type II (gyrase) | | | | Risk in fungi unknown. | |
| | | | | | Resistance management | |
| | | | | | required. | |
| | A5 | DHODHI- | phenyl-propanol | ipflufenoquin | Medium to high risk. | 52 |
| | inhibition of | fungicides | | | | |
| | dihydroorotate | | | | | |
| | dehydrogenase | | | | | |
| | within de novo | | | | | |
| | pyrimidine | | | | | |
| | biosynthesis | | | | | |
| B: | B1 | MBC - | benzimidazoles | benomyl | Resistance common in many | 1 |
| Cytoskeleton | tubulin | fungicides | | carbendazim | fungal species. Several target | |
| and motor | polymerization | (Methyl | | fuberidazole | site mutations, mostly | |
| protein | | Benzimidazole | | thiabendazole | E198A/G/K, F200Y in | |
| | | Carbamates) | thiophanates | thiophanate | β-tubulin gene. | |
| | | | | thiophanate- | Positive cross resistance | |
| | | | | methy [symbol] | between the group members. | |
| | | | | | Negative cross resistance to | |
| | | | | | N-phenyl carbamates. | |
| | | | | | High risk. | |
| | | | | | See FRAC Benzimidazole | |
| | | | | | Guidelines for resistance | |
| | | | | | management. | |
| | B2 | N-phenyl | N-phenyl | diethofencarb | Resistance known. Target site | 10 |
| | tubulin | carbamates | carbamates | | mutation E198K. Negative | |
| | polymerization | | | | cross resistance to | |
| | | | | | benzimidazoles. | |
| | | | | | High risk. | |
| | | | | | Resistance management | |
| | | | | | required. | |
| | B3 | benzamides | toluamides | zoxamide | Low to medium risk. | 22 |
| | tubulin | thiazole | ethylamino-thiazole- | ethaboxam | Resistance management | |
| | polymerization | carboxamide | carboxamide | | required. | |
| | B4 | phenylureas | phenylureas | pencycuron | Resistance not known. | 20 |
| | cell division | | | | | |
| | (unknown site) | | | | | |
| | B5 | benzamides | pyridinylmethyl- | fluopicolide | Resistant isolates detected in | 43 |
| | delocalisation of | | benzamides | fluopimomide | grapevine downy mildew. | |
| | spectrin-like | | | | Medium risk. | |
| | proteins | | | | Resistance management | |
| | | | | | required | |
| | B6 | cyanoacrylates | aminocyanoacrylates | phenamacril | Resistance known in | 47 |
| | actin/myosin/fimbrin | | | | Fusarium | |
| | function | | | | graminearum. | |
| | | | | | Target site mutations in the | |
| | | | | | gene coding for myosin-5 | |
| | | | | | found in lab studies. | |
| | | | | | Medium to high risk. | |
| | | | | | Resistance management | |
| | | | | | required. | |
| | | aryl-phenyl- | benzophenone | metrafenone | Less sensitive isolates | 50 |
| | | ketones | benzoylpyridine | pyriofenone | detected in powdery mildews | |
| | | | | | (Blumeria and Sphaerotheca) | |
| | | | | | Medium risk. | |
| | | | | | Resistance management | |
| | | | | | required. | |
| | | | | | Reclassified from U8 in 2018 | |
| | B7 | pyridazine | pyridazine | pyridachlometyl | High risk. | 53 |
| | tubulin dynamics | | | | | |
| | modulator | | | | | |
| C. | C1 | pyrimidinamines | pyrimidinamines | diflumetorim | Resistance not known. | 39 |
| respiration | complex I NADH | pyrazole-MET1 | pyrazole-5- | tolfenpyrad | | |
| | oxido-reductase | | carboxamides | | | |
| | | Quinazoline | quinazoline | fenazaquin | | |
| | C2 | SDHI | phenyl-benzamides | benodanil | Resistance known for several | 7 |
| | complex II: | (Succinate- | | flutolanil | fungal species in field | |
| | succinate-dehydro- | dehydrogenase | | mepronil | populations and lab mutants. | |
| | genase | inhibitors) | phenyl-oxo-ethyl | isofetamid | Target site mutations in sdh | |
| | | | thiophene amide | | gene, e.g. H/Y (or H/L) at 257, | |
| | | | pyridinyl-ethyl- | fluopyram | 267, 272 or P225L, dependent | |
| | | | benzamides | | on fungal species. | |
| | | | phenyl-cyclobutyl- | cyclobutrifluram | Resistance management | |
| | | | pyridineamide | | required. | |
| | | | furan- carboxamides | fenfuram | Medium to high risk. | |
| | | | oxathiin- | carboxin | See FRAC SDHI Guidelines | |
| | | | carboxamides | oxycarboxin | for resistance management. | |
| | | | thiazole- | thifluzamide | | |
| | | | carboxamides | | | |
| | | | pyrazole-4- | benzovindiflupyr | | |
| | | | carboxamides | bixafen | | |
| | | | | fluindapyr | | |
| | | | | fluxapyroxad | | |
| | | | | furametpyr | | |
| | | | | inpyrfluxam | | |
| | | | | isopyrazam | | |
| | | | | penflufen | | |
| | | | | penthiopyrad | | |
| | | | | sedaxane | | |
| | | | N-cyclopropyl-N- | isoflucypram | | |
| | | | benzyl-pyrazole- | | | |
| | | | carboxamides | | | |
| | | | N-methoxy-(phenyl- | pydiflumetofen | | |
| | | | ethyl)-pyrazole- | | | |
| | | | carboxamides | | | |
| | | | pyridine- | boscalid | | |
| | | | carboxamides | | | |
| | | | pyrazine- | pyraziflumid | | |
| | | | carboxamides | | | |
| C. | C3 | QoI-fungicides | methoxy-acrylates | azoxystrobin | Resistance known in various | 11 |
| respiration | complex III: | (Quinone outside | | coumoxystrobin | fungal species. Target site | |
| | cytochrome bc1 | Inhibitors) | | enoxastrobin | mutations in cyt b gene (G143A, | |
| | (ubiquinol oxidase) | | | flufenoxystrobin | F129L) and additional | |
| | at Qo site (cyt b | | | picoxystrobin | mechanisms. | |
| | gene) | | | pyraoxystrobin | Cross resistance shown | |
| | | | methoxy-acetamide | mandestrobin | between all members of the | |
| | | | methoxy-carbamates | pyraclostrobin | Code 11 fungicides. | |
| | | | | pyrametostrobin | High risk. | |
| | | | | triclopyricarb | See FRAC QoI Guidelines | |
| | | | oximino-acetates | kresoxim-methyl | for resistance management. | |
| | | | | trifloxystrobin | | |
| | | | oximino-acetamides | dimoxystrobin | | |
| | | | | fenaminstrobin | | |
| | | | | metominostrobin | | |
| | | | | orysastrobin | | |
| | | | oxazolidine-diones | famoxadone | | |
| | | | dihydro-dioxazines | fluoxastrobin | | |
| | | | imidazolinones | fenamidone | | |
| | | | benzyl-carbamates | pyribencarb | | |
| | | QoI-fungicides | tetrazolinones | metyltetraprole | Resistance not known. Not | 11A |
| | | (Quinone outside | | | cross resistant with Code 11 | |
| | | Inhibitors; | | | fungicides on G143A mutants. | |
| | | Subgroup A) | | | High risk. | |
| | | | | | See FRAC QoI Guidelines | |
| | | | | | for resistance management. | |
| C: | C4 | QiI - fungicides | cyano-imidazole | cyazofamid | Resistance risk unknown but | 21 |
| respiration | complex III: | (Quinone inside | sulfamoyl-triazole | amisulbrom | assumed to be medium to high | |
| (continued) | cytochrome bc1 | Inhibitors) | picolinamides | fenpicoxamid | (mutations at target site known | |
| | (ubiquinone | | | florylpicoxamid | in model organisms). | |
| | reductase) at Qi site | | | | Resistance management | |
| | | | | | required. | |
| | | | | | No spectrum overlap with the | |
| | | | | | Oomycete-fungicides | |
| | | | | | cyazofamid and amisulbrom | |
| | C5 | | dinitrophenyl- | binapacryl | Resistance not known. | 29 |
| | uncouplers of | | crotonates | meptyldinocap | Also acaricidal activity. | |
| | oxidative phos- | | | dinocap | | |
| | phorylation | | 2,6-dinitro-anilines | fluazinam | Low risk. However, resistance | |
| | | | | | claimed in Botrytis in Japan. | |
| | | | (pyr.-hydrazones) | (ferimzone) | Reclassified to U 14 in 2012. | |
| | C6 | organo tin | tri-phenyl tin | fentin acetate | Some resistance cases | 30 |
| | inhibitors of | compounds | compounds | fentin chloride | known. Low to medium risk. | |
| | oxidative phos- | | | fentin hydroxide | | |
| | phorylation, ATP | | | | | |
| | synthase | | | | | |
| | C7 | thiophene- | thiophene- | silthiofam | Resistance reported. Risk low. | 38 |
| | ATP transport | carboxamides | carboxamides | | | |
| | (proposed) | | | | | |
| | C8 | QoSI fungicides | triazolo-pyrimidylamine | ametoctradin | Not cross resistant to QoI | 45 |
| | complex III: | (Quinone outside | | | fungicides. | |
| | cytochrome bc1 | Inhibitor, | | | Resistance risk assumed to | |
| | (ubiquinone | stigmatellin | | | be medium to high | |
| | reductase) at | binding type) | | | (single site inhibitor). | |
| | Qo site, stigmatellin | | | | Resistance management | |
| | binding sub-site | | | | required. | |
| D: | D1 | AP - fungicides | anilino-pyrimidines | cyprodinil | Resistance known in Botrytis | 9 |
| amino acids | methionine | (Anilino- | | mepanipyrim | and Venturia, sporadically in | |
| and protein | biosynthesis | Pyrimidines) | | pyrimethanil | Oculimacula. | |
| [symbol] | (proposed) | | | | Medium risk. | |
| | (cgs gene) | | | | See FRAC Anilinopyrimidine | |
| | | | | | Guidelines | |
| | | | | | for resistance management. | |
| | D2 | enopyranuronic | enopyranuronic acid | blasticidin-S | Low to medium risk. | 23 |
| | protein synthesis | acid antibiotic | antibiotic | | Resistance management | |
| | (ribosome, | | | | required. | |
| | termination step) | | | | | |
| | D3 | hexopyranosyl | hexopyranosyl | kasugamycin | Resistance known in fungal | 24 |
| | protein synthesis | antibiotic | antibiotic | | and bacterial (P. glumae) | |
| | (ribosome, initiation | | | | pathogens. Medium risk. | |
| | step) | | | | Resistance management | |
| | | | | | required. | |
| | D4 | glucopyranosyl | glucopyranosyl | streptomycin | Bactericide. Resistance | 25 |
| | protein synthesis | antibiotic | antibiotic | | known. High risk. | |
| | (ribosome, initiation | | | | Resistance management | |
| | step) | | | | required. | |
| | D5 | tetracycline | tetracycline | oxytetracycline | Bactericide. Resistance | 41 |
| | protein synthesis | antibiotic | antibiotic | | known. High risk. | |
| | (ribosome, | | | | Resistance management | |
| | elongation step) | | | | required. | |
| E: | E1 | aza- | aryloxyquinoline | quinoxyfen | Resistance to quinoxyfen | 13 |
| signal | signal transduction | naphthalenes | quinazolinone | proquinazid | known. | |
| transduction | (mechanism | | | | Medium risk. | |
| | unknown) | | | | Resistance management | |
| | | | | | required. Cross resistance | |
| | | | | | found in Erysiphe (Uncinula) | |
| | | | | | necator but not in Blumeria | |
| | | | | | graminis. | |
| | E2 | PP-fungicides | phenylpyrroles | fenpiclonil | Resistance found sporadically, | 12 |
| | MAP/Histidine- | (PhenylPyrroles) | | fludioxonil | mechanism speculative. | |
| | Kinase in osmotic | | | | Low to medium risk. | |
| | signal transduction | | | | Resistance management | |
| | (os-2, HOG1) | | | | required. | |
| | E3 | dicarboximides | dicarboximides | chlozolinate | Resistance common in Botrytis | 2 |
| | MAP/Histidine- | | | dimethachlone | and some other pathogens. | |
| | Kinase in osmotic | | | iprodione | Several mutations in OS-1, | |
| | signal transduction | | | procymidone | mostly I365S. | |
| | (os-1, Daf1) | | | vinclozolin | Cross resistance common | |
| | | | | | between the group members. | |
| | | | | | Medium to high risk. | |
| | | | | | See FRAC Dicarboximide | |
| | | | | | Guidelines | |
| | | | | | for resistance management | |

| | | |
|---|---|---|
| F: lipid | F1 | formerly dicarboximides |

| | | | | | | |
|---|---|---|---|---|---|---|
| synthesis or | F2 | phosphoro- | phosphoro- | edifenphos | Resistance known in specific | 6 |
| transport/ | phospholipid | thiolates | thiolates | iprobenfos (IBP) | fungi. | |
| membrane | biosynthesis, | | | pyrazophos | Low to medium risk. | |
| integrity or | methyltransferase | Dithiolanes | dithiolanes | isoprothiolane | Resistance management | |
| function | | | | | required if used for risky | |
| | | | | | pathogens. | |
| | F3 | AH-fungicides | aromatic hydrocarbons | biphenyl | Resistance known in some | 14 |
| | cell peroxidation | (Aromatic | | chloroneb | fungi. | |
| | (proposed) | Hydrocarbons) | | dicloran | Low to medium risk. | |
| | | (chlorophenyls, | | quintozene (PCNB) | Cross resistance patterns | |
| | | nitroanilines) | | tecnazene (TCNB) | complex due to different | |
| | | | | tolclofos-methyl | activity spectra. | |
| | | heteroaromatics | 1,2,4-thiadiazoles | etridiazole | | |
| | F4 | Carbamates | carbamates | iodocarb | Low to medium risk. | 28 |
| | cell membrane | | | propamocarb | Resistance management | |
| | permeability, fatty | | | prothiocarb | required. | |
| | acids (proposed) | | | | | |

| | |
|---|---|
| F5 | formerly CAA-fungicides |
| F6 | formerly Bacillus amyloliquefaciens |
| microbial disrupters | strains (FRAC Code 44); |
| of pathogen cell | reclassified to BM02 in 2020 |
| membranes | |
| F7 | formerly extract from Melaleuca |
| cell membrane | alternifolia (tea tree oil) and plant |
| disruption | oils (eugenol, geraniol, thymol) FRAC Code 46, |
| | reclassified to BM01 in 2021 |

| | | | | | | |
|---|---|---|---|---|---|---|
| | F8 | Polyene | amphoteric macrolide | natamycin | Resistance not known. | 48 |
| | ergosterol binding | | antifungal antibiotic | (pimaricin) | Agricultural, food and topical | |
| | | | from Streptomyces | | medical uses. | |
| | | | natalensis or | | | |
| | | | S. chattanoogensis | | | |
| | F9 | OSBPI | piperidinyl-thiazole- | oxathiapiprolin | Resistance risk assumed to be | 49 |
| | lipid homeostasis | oxysterol binding | isoxazolines | fluoxapiprolin | medium to high (single site | |
| | and transfer/storage | protein | | | inhibitor). Resistance | |
| | | homologue | | | management required. | |
| | | inhibition | | | (Previously U15). | |
| | F10 | protein fragment | polypeptide | polypeptide | Resistance not known. | 51 |
| | interaction with lipid | | | ASFBIOF01-02 | | |
| | fraction of the cell | | | | | |
| | membrane, with | | | | | |
| | multiple effects on | | | | | |
| | cell membrane | | | | | |
| | integrity | | | | | |
| G: sterol | G1 | DMI-fungicides | piperazines | triforine | There are big differences in | 3 |
| biosynthesis | C14- demethylase | (DeMethylation | pyridines | pyrifenox | the activity spectra of DMI | |
| in [symbol] | in sterol | Inhibitors) | | pyrisoxazole | fungicides. | |
| | biosynthesis | (SBI: Class I) | pyrimidines | fenarimol | Resistance is known in various | |
| | (erg11/cyp51) | | | nuarimol | fungal species. Several | |
| | | | imidazoles | imazalil | resistance mechanisms are | |
| | | | | oxpoconazole | known incl. target site | |
| | | | | pefurazoate | mutations in cyp51 (erg 11) | |
| | | | | prochloraz | gene, e.g. V136A, Y137F, | |
| | | | | triflumizole | A379G, I381V; cyp51 | |
| | | | triazoles | azaconazole | promotor; ABC transporters | |
| | | | triazolinthiones | bitertanol | and others. | |
| | | | | bromuconazole | Generally wise to accept that | |
| | | | | cyproconazole | cross resistance is present | |
| | | | | difenoconazole | between DMI fungicides active | |
| | | | | diniconazole | against the same fungus. | |
| | | | | epoxiconazole | DMI fungicides are Sterol | |
| | | | | etaconazole | Biosynthesis Inhibitors (SBIs), | |
| | | | | fenbuconazole | but show no cross resistance | |
| | | | | fluquinconazole | to other SBI classes. | |
| | | | | flusilazole | Medium risk. | |
| | | | | flutriafol | See FRAC SBI Guidelines | |
| | | | | hexaconazole | for resistance management. | |
| | | | | imibenconazole | | |
| | | | | ipconazole | | |
| | | | | mefentrifluconazole | | |
| | | | | metconazole | | |
| | | | | myclobutanil | | |
| | | | | penconazole | | |
| | | | | propiconazole | | |
| | | | | simeconazole | | |
| | | | | tebuconazole | | |
| | | | | tetraconazole | | |
| | | | | triadimefon | | |
| | | | | triadimenol | | |
| | | | | triticonazole | | |
| | | | | prothioconazole | | |
| | G2 | amines | morpholines | aldimorph | Decreased sensitivity for | 5 |
| | Δ¹⁴-reductase | (“morpholines”) | | dodemorph | powdery mildews. | |
| | and | (SBI: Class II) | | fenpropimorph | Cross resistance within the | |
| | Δ⁸→Δ⁷⁻ | | | tridemorph | group generally found but not | |
| | isomerase | | piperidines | fenpropidin | to other | |
| | in sterol | | | piperalin | SBI classes | |
| | biosynthesis | | spiroketal-amines | spiroxamine | Low to medium risk. | |
| | (erg24, erg2) | | | | See FRAC SBI Guidelines | |
| | | | | | for resistance management | |
| | G3 | KRI fungicides | hydroxyanilides | fenhexamid | Low to medium risk. | 17 |
| | 3-keto reductase, | (KetoReductase | amino-pyrazolinone | fenpyrazamine | Resistance management | |
| | C4- de-methylation | Inhibitors) | | | required. | |
| | (erg27) | (SBI: Class III) | | | | |
| | G4 | (SBI class IV) | thiocarbamates | pyributicarb | Resistance not known, | 18 |
| | squalene-epoxidase | | allylamines | naftifine | fungicidal and herbicidal | |
| | in sterol | | | terbinafine | activity. | |
| | biosynthesis | | | | Medical fungicides only. | |
| | (erg1) | | | | | |

| | | | | |
|---|---|---|---|---|
| H: cell wall | H3 | Formerly glucopyranosyl | reclassified to U18 | 26 |
| biosynthesis | | antibiotic (validamycin) | | |

| | | | | | | |
|---|---|---|---|---|---|---|
| | H4 | polyoxins | peptidyl pyrimidine | polyoxin | Resistance known. | 19 |
| | chitin synthase | | nucleoside | | Medium risk. | |
| | | | | | Resistance management | |
| | | | | | required. | |
| | H5 | CAA-fungicides | cinnamic acid amides | dimethomorph | Resistance known in | 40 |
| | cellulose synthase | (Carboxylic Acid | | flumorph | Plasmopara viticola but not in | |
| | | Amides) | | pyrimorph | Phytophthora infestans. | |
| | | | valinamide | benthiavalicarb | Cross resistance between all | |
| | | | carbamates | iprovalicarb | members of the CAA group. | |
| | | | | valifenalate | Low to medium risk. | |
| | | | mandelic acid amides | mandipropamid | See FRAC CAA Guidelines for | |
| | | | | | resistance management. | |
| I: melanin | I1 | MBI-R | isobenzo-furanone | fthalide | Resistance not known. | 16.1 |
| synthesis in | reductase in | (Melanin | pyrrolo-quinolinone | pyroquilon | | |
| cell wall | melanin | Biosynthesis | triazolobenzo- | tricyclazole | | |
| | biosynthesis | Inhibitors - | thiazole | | | |
| | | Reductase) | | | | |
| | I2 | MBI-D | cyclopropane- | carpropamid | Resistance known. | 16.2 |
| | dehydratase in | (Melanin | carboxamide | | Medium risk. | |
| | melanin | Biosynthesis | carboxamide | diclocymet | Resistance management | |
| | biosynthesis | Inhibitors - | propionamide | fenoxanil | required. | |
| | | Dehydratase) | | | | |
| | I3 | MBI-P | trifluoroethyl- | tolprocarb | Resistance not known. | 16.3 |
| | polyketide synthase | (Melanin | carbamate | | Additional activity against | |
| | in melanin | Biosynthesis | | | bacteria and fungi through | |
| | biosynthesis | Inhibitors - | | | induction of host plant defence | |
| | | Polyketide | | | | |
| | | synthase) | | | | |
| P: host plant | P 01 | benzo- | benzo- | acibenzolar-S-methyl | Resistance not known. | P 01 |
| defence | salicylate-related | thiadiazole | thiadiazole | | | |
| induction | | (BTH) | (BTH) | | | |
| | P 02 | benzisothiazole | benzisothiazole | probenazole | Resistance not known. | P 02 |
| | salicylate-related | | | (also antibacterial and | | |
| | | | | antifungal activity) | | |
| | P 03 | thiadiazole- | thiadiazole- | tiadinil | Resistance not known. | P 03 |
| | salicylate-related | carboxamide | carboxamide | isotianil | | |
| | P 04 | natural | polysaccharides | laminarin | Resistance not known. | P 04 |
| | polysaccharide | compound | | | | |
| | elicitors | | | | | |
| | P 05 | plant extract | complex mixture, | extract from Reynoutria | Resistance not known. | P 05 |
| | anthraquinone | | ethanol extract | sachalinensis (giant | | |
| | elicitors | | (anthraquinones, | knotweed) | | |
| | | | resveratrol) | | | |
| | P 06 | microbial | bacterial | Bacillus mycoides | Resistance not known. | P 06 |
| | microbial elicitors | | Bacillus spp. | isolate J | | |
| | | | fungal | cell walls of Saccharomyces | | |
| | | | Saccharomyces | cerevisiae | | |
| | | | spp. | strain LAS117 | | |
| | P 07 | phosphonates | ethyl phosphonates | fosetyl-Al | Few resistance cases | P07 |
| | phosphonates | | | phosphorous acid and | reported in few | |
| | | | | salts | pathogens. | |
| | | | | | Low risk. | |
| | | | | | Reclassified from U33 in | |
| | | | | | 2018 | |
| | P 08 | isothiazole | isothiazolylmethyl | dichlobentiazox | activates SAR both up- | P 08 |
| | salicylate-related | | ether | | and downstream of SA. | |
| | | | | | Resistance not known. | |
| U: | unknown | cyanoacetamide- | cyanoacetamide- | cymoxanil | Resistance claims described. | 27 |
| Unknown | | oxime | oxime | | Low to medium risk. | |
| mode of | | | | | Resistance management | |
| action | | | | | required. | |

| | |
|---|---|
| (U numbers | formerly phosphonates (FRAC code 33), |
| not | reclassified to P 07 in 2018 |

| | | | | | | |
|---|---|---|---|---|---|---|
| appearing | unknown | phthalamic acids | phthalamic acids | tecloftalam | Resistance not known. | 34 |
| in the list | | | | (Bactericide) | | |
| derive from | unknown | benzotriazines | benzotriazines | triazoxide | Resistance not known. | 35 |
| reclassified | unknown | benzene- | benzene- | flusulfamide | Resistance not known. | 36 |
| fungicides) | unknown | pyridazinones | pyridazinones | diclomezine | Resistance not known. | 37 |

formerly methasulfocarb (FRAC code 42),
reclassified to M 12 in 2018

| | | | | | | |
|---|---|---|---|---|---|---|
| | unknown | phenyl- | phenyl- | cyflufenamid | Resistance in Sphaerotheca. | U 06 |
| | | acetamide | acetamide | | Resistance management | |
| | | | | | required | |
| | cell membrane | guanidines | guanidines | dodine | Resistance known in | U 12 |
| | disruption | | | | Venturia inaequalis. | |
| | (proposed) | | | | Low to medium risk. | |
| | | | | | Resistance management | |
| | | | | | recommended. | |
| | unknown | thiazolidine | cyano-methylene- | flutianil | Resistance in Sphaerotheca and | U 13 |
| | | | thiazolidines | | Podosphaera xanthii. | |
| | | | | | Resistance management | |
| | | | | | required. | |
| | unknown | pyrimidinone- | pyrimidinone- | ferimzone | Resistance not known | U 14 |
| | | hydrazones | hydrazones | | (previously C5). | |
| | complex III: | 4-quinolyl- | 4-quinolyl- | tebufloquin | Not cross resistant to QoI. | U 16 |
| | cytochrome bc1, | acetate | acetates | | Resistance risk unknown but | |
| | unknown binding | | | | assumed to be medium. | |
| | site (proposed) | | | | Resistance management | |
| | | | | | required. | |
| | Unknown | tetrazolyloxime | tetrazolyloximes | picarbutrazox | Resistance not known. | U 17 |
| | | | | | Not cross resistant to | |
| | | | | | PA, QoI, CAA. | |
| | Unknown | glucopyranosyl | glucopyranosyl | validamycin | Resistance not known. | U 18 |
| | (Inhibition of | antibiotic | antibiotics | | Induction of host plant defense | |
| | trehalase) | | | | by trehalose proposed | |
| | | | | | (previously H3). | |
| Not | Unknown | diverse | diverse | mineral oils, | Resistance not known. | NC |
| specified | | | | organic oils, | | |
| | | | | inorganic salts, | | |
| | | | | material of | | |
| | | | | biological origin | | |
| M: | multi-site | inorganic | inorganic | copper | Also applies to organic copper | M 01 |
| Chemicals | contact | (electrophiles) | | (different salts) | complexes | |
| with multi-site | activity | inorganic | inorganic | sulphur | generally considered as a low | M 02 |
| activity | | (electrophiles) | | | risk group without any signs of | |
| | | dithiocarbamates | dithio-carbamates | amobam | resistance developing to the | M 03 |
| | | and relatives | and relatives | ferbam | fungicides. | |
| | | (electrophiles) | | mancozeb | reclassified from U42 in 2018 | |
| | | | | maneb | | |
| | | | | metiram | | |
| | | | | propineb | | |
| | | | | thiram | | |
| | | | | zinc thiazole | | |
| | | | | zineb | | |
| | | | | ziram | | |
| | | phthalimides | phthalimides | captan | | M 04 |
| | | (electrophiles) | | captafol | | |
| | | | | folpet | | |
| | | chloronitriles | chloronitriles | chlorothalonil | | M 05 |
| | | (phthalonitriles) | (phthalonitriles) | | | |
| | | (unspecified | | | | |
| | | mechanism) | | | | |
| | | sulfamides | sulfamides | dichlofluanid | | M 06 |
| | | (electrophiles) | | tolylfluanid | | |
| | | bis-guanidines | bis-guanidines | guazatine | | M 07 |
| | | (membrane | | iminoctadine | | |
| | | disruptors, | | | | |
| | | detergents) | | | | |
| | | triazines | triazines | anilazine | | M 08 |
| | | (unspecified | | | | |
| | | mechanism) | | | | |
| | | quinones | quinones | dithianon | | M 09 |
| | | (anthraquinones) | (anthraquinones) | | | |
| | | (electrophiles) | | | | |
| | | quinoxalines | quinoxalines | chinomethionat/ | | M 10 |
| | | (electrophiles) | | quinomethionate | | |
| | | maleimide | maleimide | fluoroimide | | M 11 |
| | | (electrophiles) | | | | |
| | | thiocarbamate | thiocarbamate | methasulfocarb | | M 12 |
| | | (electrophiles) | | | | |

| | | | CHEMICAL OR | | | |
|---|---|---|---|---|---|---|
| | TARGET | GROUP | BIOLOGICAL | COMMON | | FRAC |
| MOA | SITE | NAME | GROUP | NAME | COMMENTS | CODE |
| BM: | multiple effects | plant extract | polypeptide (lectin) | extract from the | Resistance not known. | BM 01 |
| Biologicals | on ion membrane | | | cotyledons of | (previously M12). | |
| with | transporters; | | | lupine plantlets | | |
| multiple | chelating effects | | | (“BLAD”) | | |
| modes | affects fungal | plant extract | phenols, | extract from | Resistance not known. | |
| of | spores and germ | | sesquiterpenes, | Swinglea glutinosa | | |
| action: | tubes, | | triterpenoids, | | | |
| Plant | induced plant | | coumarins | | | |
| extracts | defense | | | | | |
| | cell membrane | plant extract | terpene | extract from | Resistance not known. | |
| | disruption, cell wall, | | hydrocarbons, | Melaleuca | (previously F7) | |
| | induced plant | | terpene alcohols and | alternifolia | | |
| | defense | | terpene phenols | (tea tree oil) | | |
| | mechanisms | | | plant oils | | |
| | | | | (mixtures): | | |
| | | | | eugenol, geraniol, | | |
| | | | | thymol | | |
| BM: | multiple effects | microbial | fungal | T. atroviride | nomenclature change from | BM 02 |
| Biologicals | described | (strains | Trichoderma spp. | strain I-1237 | Gliocladium catenulatum to | |
| with | (examples, not all | of living | | strain LU132 | Clonostachys rosea | |
| multiple | apply to all | microbes or | | strain SC1 | Resistance not known. | |
| modes | biological groups): | extract, | | strain SKT-1 | Bacillus amyloliquefaciens | |
| of | competition, | metabolites) | | strain 77B | reclassified from F6, | |
| action: | mycoparasitism, | | | T. asperellum | Code 44 in 2020 | |
| Microbial | antibiosis, | | | strain T34 | synonyms for Bacillus | |
| (living | membrane | | | strain kd | amyloliquefaciens are Bacillus | |
| microbes, | disruption by | | | T. harzianum | subtilis and B. subtilis var. | |
| extracts | fungicidal | | | strain T-22 | amyloliquefaciens (previous | |
| or | lipopeptides, | | | T. virens | taxonomic classification). | |
| metabolites) | lytic enzymes, | | | strain G-41 | | |
| | induced plant | | fungal | C. rosea | | |
| | defence | | Clonostachys spp. | strain J1446 | | |
| | | | | strain CR-7 | | |
| | | | fungal | C. minitans | | |
| | | | Coniothyrium spp. | strain CON/M/91-08 | | |
| | | | fungal | H. uvarum | | |
| | | | Hanseniaspora spp. | strain BC18Y | | |
| | | | fungal | T. flavus | | |
| | | | Talaromyces spp. | strain SAY-Y-94-01 | | |
| | | | fungal | S. cerevisae | | |
| | | | Saccharomyces spp. | strain LAS02 | | |
| | | | | strain DDSF623 | | |
| | | | bacterial | B. amyloliquefaciens | | |
| | | | Bacillus spp. | strain QST713 | | |
| | | | | strain FZB24 | | |
| | | | | strain MBI600 | | |
| | | | | strain D747 | | |
| | | | | strain F727 | | |
| | | | | strain AT-332 | | |
| | | | | B. subtilis | | |
| | | | | strain AFS032321 | | |
| | | | | strain Y1336 | | |
| | | | | strain HAI-0404 | | |
| | | | bacterial | PHC25279 | | |
| | | | Erwinia spp. | | | |
| | | | (peptide) | | | |
| | | | bacterial | G. cerinus | | |
| | | | Gluconobacter spp. | strain BC18B | | |
| | | | bacterial | P. chlororaphis | | |
| | | | Pseudomonas spp. | strain AFS009 | | |
| | | | bacterial | S. griseovirides | | |
| | | | Streptomyces spp. | strain K61 | | |
| | | | | S. lydicus | | |
| | | | | strain WYEC108 | | |

[symbol]

indicates data missing or illegible when filed

APPENDIX 2

| MODE OF ACTION | CHEMICAL CLASSIFICATION | ACTIVE |
|---|---|---|
| Inhibition of Acetyl CoA | Cyclohexanediones (DIMs) | Alloxydim |
| Carboxylase | | |
| Inhibition of Acetyl CoA | Cyclohexanediones (DIMs) | Butroxydim |
| Carboxylase | | |
| Inhibition of Acetyl CoA | Cyclohexanediones (DIMs) | Clethodim |
| Carboxylase | | |
| Inhibition of Acetyl CoA | Cyclohexanediones (DIMs) | Cloproxydim |
| Carboxylase | | |
| Inhibition of Acetyl CoA | Cyclohexanediones (DIMs) | Cycloxydim |
| Carboxylase | | |
| Inhibition of Acetyl CoA | Cyclohexanediones (DIMs) | Profoxydim |
| Carboxylase | | |
| Inhibition of Acetyl CoA | Cyclohexanediones (DIMs) | Sethoxydim |
| Carboxylase | | |
| Inhibition of Acetyl CoA | Cyclohexanediones (DIMs) | Tepraloxydim |
| Carboxylase | | |
| Inhibition of Acetyl CoA | Cyclohexanediones (DIMs) | Tralkoxydim |
| Carboxylase | | |
| Inhibition of Acetyl CoA | Aryloxyphenoxy-propionates | Clodinafop-propargyl |
| Carboxylase | (FOPs) | |
| Inhibition of Acetyl CoA | Aryloxyphenoxy-propionates | Clofop |
| Carboxylase | (FOPs) | |
| Inhibition of Acetyl CoA | Aryloxyphenoxy-propionates | Cyhalofop-butyl |
| Carboxylase | (FOPs) | |
| Inhibition of Acetyl CoA | Aryloxyphenoxy-propionates | Diclofop-methyl |
| Carboxylase | (FOPs) | |
| Inhibition of Acetyl CoA | Aryloxyphenoxy-propionates | Fenoxaprop-ethyl |
| Carboxylase | (FOPs) | |
| Inhibition of Acetyl CoA | Aryloxyphenoxy-propionates | Fenthiaprop |
| Carboxylase | (FOPs) | |
| Inhibition of Acetyl CoA | Aryloxyphenoxy-propionates | Fluazifop-butyl |
| Carboxylase | (FOPs) | |
| Inhibition of Acetyl CoA | Aryloxyphenoxy-propionates | Haloxyfop-methyl |
| Carboxylase | (FOPs) | |
| Inhibition of Acetyl CoA | Aryloxyphenoxy-propionates | Isoxapyrifop |
| Carboxylase | (FOPs) | |
| Inhibition of Acetyl CoA | Aryloxyphenoxy-propionates | Metamifop |
| Carboxylase | (FOPs) | |
| Inhibition of Acetyl CoA | Aryloxyphenoxy-propionates | Quizalofop-ethyl |
| Carboxylase | (FOPs) | |
| Inhibition of Acetyl CoA | Phenylpyrazoline | Pinoxaden |
| Carboxylase | | |
| Inhibition of Acetolactate | Pyrimidinyl benzoates | Bispyribac-sodium |
| Synthase | | |
| Inhibition of Acetolactate | Pyrimidinyl benzoates | Pyribenzoxim (prodrug of |
| Synthase | | bispyribac) |
| Inhibition of Acetolactate | Pyrimidinyl benzoates | Pyriftalid |
| Synthase | | |
| Inhibition of Acetolactate | Pyrimidinyl benzoates | Pyriminobac-methyl |
| Synthase | | |
| Inhibition of Acetolactate | Pyrimidinyl benzoates | Pyrithiobac-sodium |
| Synthase | | |
| Inhibition of Acetolactate | Sulfonanilides | Pyrimisulfan |
| Synthase | | |
| Inhibition of Acetolactate | Sulfonanilides | Triafamone |
| Synthase | | |
| Inhibition of Acetolactate | Triazolopyrimidine - Type 1 | Cloransulam-methyl |
| Synthase | | |
| Inhibition of Acetolactate | Triazolopyrimidine - Type 1 | Diclosulam |
| Synthase | | |
| Inhibition of Acetolactate | Triazolopyrimidine - Type 1 | Florasulam |
| Synthase | | |
| Inhibition of Acetolactate | Triazolopyrimidine - Type 1 | Flumetsulam |
| Synthase | | |
| Inhibition of Acetolactate | Triazolopyrimidine - Type 1 | Metosulam |
| Synthase | | |
| Inhibition of Acetolactate | Triazolopyrimidine - Type 2 | Penoxsulam |
| Synthase | | |
| Inhibition of Acetolactate | Triazolopyrimidine - Type 2 | Pyroxsulam |
| Synthase | | |
| Inhibition of Acetolactate | Sulfonylureas | Amidosulfuron |
| Synthase | | |
| Inhibition of Acetolactate | Sulfonylureas | Azimsulfuron |
| Synthase | | |
| Inhibition of Acetolactate | Sulfonylureas | Bensulfuron-methyl |
| Synthase | | |
| Inhibition of Acetolactate | Sulfonylureas | Chlorimuron-ethyl |
| Synthase | | |
| Inhibition of Acetolactate | Sulfonylureas | Chlorsulfuron |
| Synthase | | |
| Inhibition of Acetolactate | Sulfonylureas | Cinosulfuron |
| Synthase | | |
| Inhibition of Acetolactate | Sulfonylureas | Cyclosulfamuron |
| Synthase | | |
| Inhibition of Acetolactate | Sulfonylureas | Ethametsulfuron-methyl |
| Synthase | | |
| Inhibition of Acetolactate | Sulfonylureas | Ethoxysulfuron |
| Synthase | | |
| Inhibition of Acetolactate | Sulfonylureas | Flazasulfuron |
| Synthase | | |
| Inhibition of Acetolactate | Sulfonylureas | Flucetosulfuron |
| Synthase | | |
| Inhibition of Acetolactate | Sulfonylureas | Flupyrsulfuron-methyl-Na |
| Synthase | | |
| Inhibition of Acetolactate | Sulfonylureas | Foramsulfuron |
| Synthase | | |
| Inhibition of Acetolactate | Sulfonylureas | Halosulfuron-methyl |
| Synthase | | |
| Inhibition of Acetolactate | Sulfonylureas | Imazosulfuron |
| Synthase | | |
| Inhibition of Acetolactate | Sulfonylureas | lodosulfuron-methyl-Na |
| Synthase | | |
| Inhibition of Acetolactate | Sulfonylureas | Mesosulfuron-methyl |
| Synthase | | |
| Inhibition of Acetolactate | Sulfonylureas | Metazosulfuron |
| Synthase | | |
| Inhibition of Acetolactate | Sulfonylureas | Metsulfuron-methyl |
| Synthase | | |
| Inhibition of Acetolactate | Sulfonylureas | Nicosulfuron |
| Synthase | | |
| Inhibition of Acetolactate | Sulfonylureas | Orthosulfamuron |
| Synthase | | |
| Inhibition of Acetolactate | Sulfonylureas | Oxasulfuron |
| Synthase | | |
| Inhibition of Acetolactate | Sulfonylureas | Primisulfuron-methyl |
| Synthase | | |
| Inhibition of Acetolactate | Sulfonylureas | Propyrisulfuron |
| Synthase | | |
| Inhibition of Acetolactate | Sulfonylureas | Prosulfuron |
| Synthase | | |
| Inhibition of Acetolactate | Sulfonylureas | Pyrazosulfuron-ethyl |
| Synthase | | |
| Inhibition of Acetolactate | Sulfonylureas | Rimsulfuron |
| Synthase | | |
| Inhibition of Acetolactate | Sulfonylureas | Sulfometuron-methyl |
| Synthase | | |
| Inhibition of Acetolactate | Sulfonylureas | Sulfosulfuron |
| Synthase | | |
| Inhibition of Acetolactate | Sulfonylureas | Triasulfuron |
| Synthase | | |
| Inhibition of Acetolactate | Sulfonylureas | Tribenuron-methyl |
| Synthase | | |
| Inhibition of Acetolactate | Sulfonylureas | Thifensulfuron-methyl |
| Synthase | | |
| Inhibition of Acetolactate | Sulfonylureas | Trifloxysulfuron-Na |
| Synthase | | |
| Inhibition of Acetolactate | Sulfonylureas | Triflusulfuron-methyl |
| Synthase | | |
| Inhibition of Acetolactate | Sulfonylureas | Tritosulfuron |
| Synthase | | |
| Inhibition of Acetolactate | Imidazolinones | Imazamethabenz-methyl |
| Synthase | | |
| Inhibition of Acetolactate | Imidazolinones | Imazamox |
| Synthase | | |
| Inhibition of Acetolactate | Imidazolinones | Imazapic |
| Synthase | | |
| Inhibition of Acetolactate | Imidazolinones | Imazapyr |
| Synthase | | |
| Inhibition of Acetolactate | Imidazolinones | Imazaquin |
| Synthase | | |
| Inhibition of Acetolactate | Imidazolinones | Imazethapyr |
| Synthase | | |
| Inhibition of Acetolactate | Triazolinones | Flucarbazone-Na |
| Synthase | | |
| Inhibition of Acetolactate | Triazolinones | Propoxycarbazone-Na |
| Synthase | | |
| Inhibition of Acetolactate | Triazolinones | Thiencarbazone-methyl |
| Synthase | | |
| Inhbition of Photosynthesis at | Triazines | Atraton |
| PSII - Serine 264 Binders | | |
| Inhbition of Photosynthesis at | Triazines | Atrazine |
| PSII - Serine 264 Binders | | |
| Inhbition of Photosynthesis at | Triazines | Ametryne |
| PSII - Serine 264 Binders | | |
| Inhbition of Photosynthesis at | Triazines | Aziprotryne = aziprotryn |
| PSII - Serine 264 Binders | | |
| Inhbition of Photosynthesis at | Triazines | Chlorazine |
| PSII - Serine 264 Binders | | |
| Inhbition of Photosynthesis at | Triazines | CP 17029 |
| PSII - Serine 264 Binders | | |
| Inhbition of Photosynthesis at | Triazines | Cyanazine |
| PSII - Serine 264 Binders | | |
| Inhbition of Photosynthesis at | Triazines | Cyprazine |
| PSII - Serine 264 Binders | | |
| Inhbition of Photosynthesis at | Triazines | Desmetryne |
| PSII - Serine 264 Binders | | |
| Inhbition of Photosynthesis at | Triazines | Dimethametryn |
| PSII - Serine 264 Binders | | |
| Inhbition of Photosynthesis at | Triazines | Dipropetryn |
| PSII - Serine 264 Binders | | |
| Inhbition of Photosynthesis at | Triazines | Eglinazine-ethyl |
| PSII - Serine 264 Binders | | |
| Inhbition of Photosynthesis at | Triazines | Ipazine |
| PSII - Serine 264 Binders | | |
| Inhbition of Photosynthesis at | Triazines | Methoprotryne = methoprotryn |
| PSII - Serine 264 Binders | | |
| Inhbition of Photosynthesis at | Triazines | procyazine |
| PSII - Serine 264 Binders | | |
| Inhbition of Photosynthesis at | Triazines | Proglinazine-ethyl |
| PSII - Serine 264 Binders | | |
| Inhbition of Photosynthesis at | Triazines | Prometon |
| PSII - Serine 264 Binders | | |
| Inhbition of Photosynthesis at | Triazines | Prometryne |
| PSII - Serine 264 Binders | | |
| Inhbition of Photosynthesis at | Triazines | Propazine |
| PSII - Serine 264 Binders | | |
| Inhbition of Photosynthesis at | Triazines | Sebuthylazine |
| PSII - Serine 264 Binders | | |
| Inhbition of Photosynthesis at | Triazines | Secbumeton |
| PSII - Serine 264 Binders | | |
| Inhbition of Photosynthesis at | Triazines | Simetryne |
| PSII - Serine 264 Binders | | |
| Inhbition of Photosynthesis at | Triazines | Simazine |
| PSII - Serine 264 Binders | | |
| Inhbition of Photosynthesis at | Triazines | Terbumeton |
| PSII - Serine 264 Binders | | |
| Inhbition of Photosynthesis at | Triazines | Terbuthylazine |
| PSII - Serine 264 Binders | | |
| Inhbition of Photosynthesis at | Triazines | Terbutryne |
| PSII - Serine 264 Binders | | |
| Inhbition of Photosynthesis at | Triazines | Trietazine |
| PSII - Serine 264 Binders | | |
| Inhbition of Photosynthesis at | Triazolinone | Amicarbazone |
| PSII - Serine 264 Binders | | |
| Inhbition of Photosynthesis at | Triazinones | Ethiozin |
| PSII - Serine 264 Binders | | |
| Inhbition of Photosynthesis at | Triazinones | Hexazinone |
| PSII - Serine 264 Binders | | |
| Inhbition of Photosynthesis at | Triazinones | Isomethiozin |
| PSII - Serine 264 Binders | | |
| Inhbition of Photosynthesis at | Triazinones | Metamitron |
| PSII - Serine 264 Binders | | |
| Inhbition of Photosynthesis at | Triazinones | Metribuzin |
| PSII - Serine 264 Binders | | |
| Inhbition of Photosynthesis at | Uracils | Bromacil |
| PSII - Serine 264 Binders | | |
| Inhbition of Photosynthesis at | Uracils | Isocil |
| PSII - Serine 264 Binders | | |
| Inhbition of Photosynthesis at | Uracils | Lenacil |
| PSII - Serine 264 Binders | | |
| Inhbition of Photosynthesis at | Uracils | Terbacil |
| PSII - Serine 264 Binders | | |
| Inhbition of Photosynthesis at | Phenlcarbamates | Chlorprocarb |
| PSII - Serine 264 Binders | | |
| Inhbition of Photosynthesis at | Phenlcarbamates | Desmedipham |
| PSII - Serine 264 Binders | | |
| Inhbition of Photosynthesis at | Phenlcarbamates | Phenisopham |
| PSII - Serine 264 Binders | | |
| Inhbition of Photosynthesis at | Phenlcarbamates | Phenmedipham |
| PSII - Serine 264 Binders | | |
| Inhbition of Photosynthesis at | Pyridazinone | Chloridazon (=pyrazon) |
| PSII - Serine 264 Binders | | |
| Inhbition of Photosynthesis at | Pyridazinone | Brompyrazon |
| PSII - Serine 264 Binders | | |
| Inhbition of Photosynthesis at | Ureas | Benzthiazuron |
| PSII - Serine 264 Binders | | |
| Inhbition of Photosynthesis at | Ureas | Bromuron |
| PSII - Serine 264 Binders | | |
| Inhbition of Photosynthesis at | Ureas | Buturon |
| PSII - Serine 264 Binders | | |
| Inhbition of Photosynthesis at | Ureas | Chlorbromuron |
| PSII - Serine 264 Binders | | |
| Inhbition of Photosynthesis at | Ureas | Chlorotoluron |
| PSII - Serine 264 Binders | | |
| Inhbition of Photosynthesis at | Ureas | Chloroxuron |
| PSII - Serine 264 Binders | | |
| Inhbition of Photosynthesis at | Ureas | Difenoxuron |
| PSII - Serine 264 Binders | | |
| Inhbition of Photosynthesis at | Ureas | Dimefuron |
| PSII - Serine 264 Binders | | |
| Inhbition of Photosynthesis at | Ureas | Diuron |
| PSII - Serine 264 Binders | | |
| Inhbition of Photosynthesis at | Ureas | Ethidimuron |
| PSII - Serine 264 Binders | | |
| Inhbition of Photosynthesis at | Ureas | Fenuron |
| PSII - Serine 264 Binders | | |
| Inhbition of Photosynthesis at | Ureas | Fluometuron |
| PSII - Serine 264 Binders | | |
| Inhbition of Photosynthesis at | Ureas | Fluothiuron |
| PSII - Serine 264 Binders | | |
| Inhbition of Photosynthesis at | Ureas | Isoproturon |
| PSII - Serine 264 Binders | | |
| Inhbition of Photosynthesis at | Ureas | Isouron |
| PSII - Serine 264 Binders | | |
| Inhbition of Photosynthesis at | Ureas | Linuron |
| PSII - Serine 264 Binders | | |
| Inhbition of Photosynthesis at | Ureas | Metobenzuron |
| PSII - Serine 264 Binders | | |
| Inhbition of Photosynthesis at | Ureas | Metobromuron |
| PSII - Serine 264 Binders | | |
| Inhbition of Photosynthesis at | Ureas | Methabenzthiazuron |
| PSII - Serine 264 Binders | | |
| Inhbition of Photosynthesis at | Ureas | Metoxuron |
| PSII - Serine 264 Binders | | |
| Inhbition of Photosynthesis at | Ureas | Monolinuron |
| PSII - Serine 264 Binders | | |
| Inhbition of Photosynthesis at | Ureas | Monuron |
| PSII - Serine 264 Binders | | |
| Inhbition of Photosynthesis at | Ureas | Neburon |
| PSII - Serine 264 Binders | | |
| Inhbition of Photosynthesis at | Ureas | Parafluron |
| PSII - Serine 264 Binders | | |
| Inhbition of Photosynthesis at | Ureas | Siduron |
| PSII - Serine 264 Binders | | |
| Inhbition of Photosynthesis at | Ureas | Tebuthiuron |
| PSII - Serine 264 Binders | | |
| Inhbition of Photosynthesis at | Ureas | |
| PSII - Serine 264 Binders | | Thiazafluron |
| Inhbition of Photosynthesis at | Amides | |
| PSII - Serine 264 Binders | | Chloranocryl = dicryl |
| Inhbition of Photosynthesis at | Amides | Pentanochlor |
| PSII - Serine 264 Binders | | |
| Inhbition of Photosynthesis at | Amides | Propanil |
| PSII - Serine 264 Binders | | |
| Inhbition of Photosynthesis at | Nitriles | Bromofenoxim |
| PSII - Histidine 215 Binders | | |
| Inhbition of Photosynthesis at | Nitriles | Bromoxynil |
| PSII - Histidine 215 Binders | | |
| Inhbition of Photosynthesis at | Nitriles | Ioxynil |
| PSII - Histidine 215 Binders | | |
| Inhbition of Photosynthesis at | Phenyl-pyridazines | Pyridate |
| PSII - Histidine 215 Binders | | |
| Inhbition of Photosynthesis at | Benzothiadiazinone | Bentazon |
| PSII - Histidine 215 Binders | | |
| PS I Electron Diversion | Pyridiniums | Cyperquat |
| PS I Electron Diversion | Pyridiniums | Diquat |
| PS I Electron Diversion | Pyridiniums | Morfamquat |
| PS I Electron Diversion | Pyridiniums | Paraquat |
| Inhibition of | Diphenyl ethers | Lactofen |
| Protoporphyrinogen Oxidase | | |
| Inhibition of | Diphenyl ethers | Acifluorfen |
| Protoporphyrinogen Oxidase | | |
| Inhibition of | Diphenyl ethers | Bifenox |
| Protoporphyrinogen Oxidase | | |
| Inhibition of | Diphenyl ethers | Chlornitrofen |
| Protoporphyrinogen Oxidase | | |
| Inhibition of | Diphenyl ethers | Fomesafen |
| Protoporphyrinogen Oxidase | | |
| Inhibition of | Diphenyl ethers | Fluorodifen |
| Protoporphyrinogen Oxidase | | |
| Inhibition of | Diphenyl ethers | Fluoroglycofen-ethyl |
| Protoporphyrinogen Oxidase | | |
| Inhibition of | Diphenyl ethers | Fluoronitrofen |
| Protoporphyrinogen Oxidase | | |
| Inhibition of | Diphenyl ethers | Nitrofen |
| Protoporphyrinogen Oxidase | | |
| Inhibition of | Diphenyl ethers | Oxyfluorfen |
| Protoporphyrinogen Oxidase | | |
| Inhibition of | Diphenyl ethers | Chlomethoxyfen |
| Protoporphyrinogen Oxidase | | |
| Inhibition of | Phenylpyrazoles | Pyraflufen-ethyl |
| Protoporphyrinogen Oxidase | | |
| Inhibition of | N-Phenyl-oxadiazolones | Oxadiargyl |
| Protoporphyrinogen Oxidase | | |
| Inhibition of | N-Phenyl-oxadiazolones | Oxadiazon |
| Protoporphyrinogen Oxidase | | |
| Inhibition of | N-Phenyl-triazolinones | Azafenidin |
| Protoporphyrinogen Oxidase | | |
| Inhibition of | N-Phenyl-triazolinones | Carfentrazone-ethyl |
| Protoporphyrinogen Oxidase | | |
| Inhibition of | N-Phenyl-triazolinones | Sulfentrazone |
| Protoporphyrinogen Oxidase | | |
| Inhibition of | N-Phenyl-imides (procide acitive | Fluthiacet-methyl |
| Protoporphyrinogen Oxidase | form) | |
| Inhibition of | N-Phenyl-imides | Butafenacil |
| Protoporphyrinogen Oxidase | | |
| Inhibition of | N-Phenyl-imides | Saflufenacil |
| Protoporphyrinogen Oxidase | | |
| Inhibition of | N-Phenyl-imides | Pentoxazone |
| Protoporphyrinogen Oxidase | | |
| Inhibition of | N-Phenyl-imides | Chlorphthalim |
| Protoporphyrinogen Oxidase | | |
| Inhibition of | N-Phenyl-imides | Cinidon-ethyl |
| Protoporphyrinogen Oxidase | | |
| Inhibition of | N-Phenyl-imides | Flumiclorac-pentyl |
| Protoporphyrinogen Oxidase | | |
| Inhibition of | N-Phenyl-imides | Flumioxazin |
| Protoporphyrinogen Oxidase | | |
| Inhibition of | N-Phenyl-imides | Flumipropyn |
| Protoporphyrinogen Oxidase | | |
| Inhibition of | N-Phenyl-imides | Trifludimoxazin |
| Protoporphyrinogen Oxidase | | |
| Inhibition of | N-Phenyl-imides | Tiafenacil |
| Protoporphyrinogen Oxidase | | |
| Inhibition of | Other | Pyraclonil |
| Protoporphyrinogen Oxidase | | |
| Inhibition of Phytoene | Phenyl ethers | Beflubutamid |
| Desaturase | | |
| Inhibition of Phytoene | Phenyl ethers | Diflufenican |
| Desaturase | | |
| Inhibition of Phytoene | Phenyl ethers | Picolinafen |
| Desaturase | | |
| Inhibition of Phytoene | N-Phenyl heterocycles | Flurochloridone |
| Desaturase | | |
| Inhibition of Phytoene | N-Phenyl heterocycles | Norflurazon |
| Desaturase | | |
| Inhibition of Phytoene | Diphenyl heterocycles | Fluridone |
| Desaturase | | |
| Inhibition of Phytoene | Diphenyl heterocycles | Flurtamone |
| Desaturase | | |
| Inhibition of Hydroxyphenyl | Triketones | Mesotrione |
| Pyruvate Dioxygenase | | |
| Inhibition of Hydroxyphenyl | Triketones | Sulcotrione |
| Pyruvate Dioxygenase | | |
| Inhibition of Hydroxyphenyl | Triketones | Tembotrione |
| Pyruvate Dioxygenase | | |
| Inhibition of Hydroxyphenyl | Triketones | Tefuryltrione |
| Pyruvate Dioxygenase | | |
| Inhibition of Hydroxyphenyl | Triketones | Bicyclopyrone |
| Pyruvate Dioxygenase | | |
| Inhibition of Hydroxyphenyl | Triketones | Fenquinotrione |
| Pyruvate Dioxygenase | | |
| Inhibition of Hydroxyphenyl | Triketones (procide) | Benzobicyclon |
| Pyruvate Dioxygenase | | |
| Inhibition of Hydroxyphenyl | Pyrazoles (procide) | Benzofenap |
| Pyruvate Dioxygenase | | |
| Inhibition of Hydroxyphenyl | Pyrazoles | Pyrasulfotole |
| Pyruvate Dioxygenase | | |
| Inhibition of Hydroxyphenyl | Pyrazoles | Topramezone |
| Pyruvate Dioxygenase | | |
| Inhibition of Hydroxyphenyl | Pyrazoles (procide) | Pyrazolynate |
| Pyruvate Dioxygenase | | |
| Inhibition of Hydroxyphenyl | Pyrazoles (procide) | Pyrazoxyfen |
| Pyruvate Dioxygenase | | |
| Inhibition of Hydroxyphenyl | Pyrazoles | Tolpyralate |
| Pyruvate Dioxygenase | | |
| Inhibition of Hydroxyphenyl | Isoxazoles | Isoxaflutole |
| Pyruvate Dioxygenase | | |
| Inhibition of Hydroxyphenyl | Phenoxypyridazine | Cyclopyrimorate |
| Pyruvate Dioxygenase | | |
| Inhibition of Hydroxyphenyl | Isoxazolidinone | Clomazone |
| Pyruvate Dioxygenase | | |
| Inhibition of Hydroxyphenyl | Isoxazolidinone | Bixlozone |
| Pyruvate Dioxygenase | | |
| Inhibition of Hydroxyphenyl | Glycine | Glyphosate |
| Pyruvate Dioxygenase | | |
| Inhibition of Hydroxyphenyl | Phosphinic acids | Glufosinate-ammonium |
| Pyruvate Dioxygenase | | |
| Inhibition of Hydroxyphenyl | Phosphinic acids | Bialaphos/bilanafos |
| Pyruvate Dioxygenase | | |
| Inhibition of Hydroxyphenyl | Carbamate | Asulam |
| Pyruvate Dioxygenase | | |
| Inhibition of Hydroxyphenyl | Dinitroanilines | Benefin = benfluralin |
| Pyruvate Dioxygenase | | |
| Inhibition of Hydroxyphenyl | Dinitroanilines | Butralin |
| Pyruvate Dioxygenase | | |
| Inhibition of Hydroxyphenyl | Dinitroanilines | Dinitramine |
| Pyruvate Dioxygenase | | |
| Inhibition of Hydroxyphenyl | Dinitroanilines | Ethalfluralin |
| Pyruvate Dioxygenase | | |
| Inhibition of Hydroxyphenyl | Dinitroanilines | Fluchloralin |
| Pyruvate Dioxygenase | | |
| Inhibition of Hydroxyphenyl | Dinitroanilines | Isopropalin |
| Pyruvate Dioxygenase | | |
| Inhibition of Microtubule | Dinitroanilines | Nitralin |
| Assembly | | |
| Inhibition of Microtubule | Dinitroanilines | Prodiamine |
| Assembly | | |
| Inhibition of Microtubule | Dinitroanilines | Profluralin |
| Assembly | | |
| Inhibition of Microtubule | Dinitroanilines | Oryzalin |
| Assembly | | |
| Inhibition of Microtubule | Dinitroanilines | Pendimethalin |
| Assembly | | |
| Inhibition of Microtubule | Dinitroanilines | Trifluralin |
| Assembly | | |
| Inhibition of Microtubule | Pyridines | Dithiopyr |
| Assembly | | |
| Inhibition of Microtubule | Pyridines | Thiazopyr |
| Assembly | | |
| Inhibition of Microtubule | Phosphoroamidates | Butamifos |
| Assembly | | |
| Inhibition of Microtubule | Phosphoroamidates | DMPA |
| Assembly | | |
| Inhibition of Microtubule | Benzoic acid | Chlorthal-dimethyl = DCPA |
| Assembly | | |
| Inhibition of Microtubule | Benzamides | Propyzamide = pronamide |
| Assembly | | |
| Inhibition of Microtubule | Carbamates | Barban |
| Organization | | |
| Inhibition of Microtubule | Carbamates | Carbetamide |
| Organization | | |
| Inhibition of Microtubule | Carbamates | Chlorbufam |
| Organization | | |
| Inhibition of Microtubule | Carbamates | Chlorpropham |
| Organization | | |
| Inhibition of Microtubule | Carbamates | Propham |
| Organization | | |
| Inhibition of Microtubule | Carbamates | Swep |
| Organization | | |
| Inhibition of Cellulose | Triazolocarboxamide | Flupoxam |
| Synthesis | | |
| Inhibition of Cellulose | Benzamides | Isoxaben |
| Synthesis | | |
| Inhibition of Cellulose | Alkylazines | Triaziflam |
| Synthesis | | |
| Inhibition of Cellulose | Alkylazines | Indaziflam |
| Synthesis | | |
| Inhibition of Cellulose | Nitriles | Dichlobenil |
| Synthesis | | |
| Inhibition of Cellulose | Nitriles | Chlorthiamid |
| Synthesis | | |
| Uncouplers | Dinitrophenols | Dinosam |
| Uncouplers | Dinitrophenols | Dinoseb |
| Uncouplers | Dinitrophenols | DNOC |
| Uncouplers | Dinitrophenols | Dinoterb |
| Uncouplers | Dinitrophenols | Etinofen |
| Uncouplers | Dinitrophenols | Medinoterb |
| Inhibition of Very Long-Chain | Azolyl-carboxamides | Cafenstrole |
| Fatty Acid Synthesis | | |
| Inhibition of Very Long-Chain | Azolyl-carboxamides | Fentrazamide |
| Fatty Acid Synthesis | | |
| Inhibition of Very Long-Chain | Azolyl-carboxamides | Ipfencarbazone |
| Fatty Acid Synthesis | | |
| Inhibition of Very Long-Chain | α-Thioacetamides | Anilofos |
| Fatty Acid Synthesis | | |
| Inhibition of Very Long-Chain | α-Thioacetamides | Piperophos |
| Fatty Acid Synthesis | | |
| Inhibition of Very Long-Chain | Isoxazolines | Pyroxasulfone |
| Fatty Acid Synthesis | | |
| Inhibition of Very Long-Chain | Isoxazolines | Fenoxasulfone |
| Fatty Acid Synthesis | | |
| Inhibition of Very Long-Chain | Oxiranes | Indanofan |
| Fatty Acid Synthesis | | |
| Inhibition of Very Long-Chain | Oxiranes | Tridiphane |
| Fatty Acid Synthesis | | |
| Inhibition of Very Long-Chain | α-Chloroacetamides | Acetochlor |
| Fatty Acid Synthesis | | |
| Inhibition of Very Long-Chain | α-Chloroacetamides | Alachlor |
| Fatty Acid Synthesis | | |
| Inhibition of Very Long-Chain | α-Chloroacetamides | Allidochlor = CDAA |
| Fatty Acid Synthesis | | |
| Inhibition of Very Long-Chain | α-Chloroacetamides | Butachlor |
| Fatty Acid Synthesis | | |
| Inhibition of Very Long-Chain | α-Chloroacetamides | Butenachlor |
| Fatty Acid Synthesis | | |
| Inhibition of Very Long-Chain | α-Chloroacetamides | Delachlor |
| Fatty Acid Synthesis | | |
| Inhibition of Very Long-Chain | α-Chloroacetamides | Diethatyl-ethyl |
| Fatty Acid Synthesis | | |
| Inhibition of Very Long-Chain | α-Chloroacetamides | Dimethachlor |
| Fatty Acid Synthesis | | |
| Inhibition of Very Long-Chain | α-Chloroacetamides | Dimethenamid |
| Fatty Acid Synthesis | | |
| Inhibition of Very Long-Chain | α-Chloroacetamides | Metazachlor |
| Fatty Acid Synthesis | | |
| Inhibition of Very Long-Chain | α-Chloroacetamides | Metolachlor |
| Fatty Acid Synthesis | | |
| Inhibition of Very Long-Chain | α-Chloroacetamides | Pethoxamid |
| Fatty Acid Synthesis | | |
| Inhibition of Very Long-Chain | α-Chloroacetamides | Pretilachlor |
| Fatty Acid Synthesis | | |
| Inhibition of Very Long-Chain | α-Chloroacetamides | Propachlor |
| Fatty Acid Synthesis | | |
| Inhibition of Very Long-Chain | α-Chloroacetamides | Propisochlor |
| Fatty Acid Synthesis | | |
| Inhibition of Very Long-Chain | α-Chloroacetamides | Prynachlor |
| Fatty Acid Synthesis | | |
| Inhibition of Very Long-Chain | α-Chloroacetamides | Thenylchlor |
| Fatty Acid Synthesis | | |
| Inhibition of Very Long-Chain | a-Oxyacetamides | Mefenacet |
| Fatty Acid Synthesis | | |
| Inhibition of Very Long-Chain | a-Oxyacetamides | Flufenacet |
| Fatty Acid Synthesis | | |
| Inhibition of Very Long-Chain | Thiocarbamates | Butylate |
| Fatty Acid Synthesis | | |
| Inhibition of Very Long-Chain | Thiocarbamates | Cycloate |
| Fatty Acid Synthesis | | |
| Inhibition of Very Long-Chain | Thiocarbamates | Dimepiperate |
| Fatty Acid Synthesis | | |
| Inhibition of Very Long-Chain | Thiocarbamates | EPTC |
| Fatty Acid Synthesis | | |
| Inhibition of Very Long-Chain | Thiocarbamates | Esprocarb |
| Fatty Acid Synthesis | | |
| Inhibition of Very Long-Chain | Thiocarbamates | Molinate |
| Fatty Acid Synthesis | | |
| Inhibition of Very Long-Chain | Thiocarbamates | Orbencarb |
| Fatty Acid Synthesis | | |
| Inhibition of Very Long-Chain | Thiocarbamates | Pebulate |
| Fatty Acid Synthesis | | |
| Inhibition of Very Long-Chain | Thiocarbamates | Prosulfocarb |
| Fatty Acid Synthesis | | |
| Inhibition of Very Long-Chain | Thiocarbamates | Thiobencarb (=Benthiocarb) |
| Fatty Acid Synthesis | | |
| Inhibition of Very Long-Chain | Thiocarbamates | Tiocarbazil |
| Fatty Acid Synthesis | | |
| Inhibition of Very Long-Chain | Thiocarbamates | Tri-allate |
| Fatty Acid Synthesis | | |
| Inhibition of Very Long-Chain | Thiocarbamates | Vernolate |
| Fatty Acid Synthesis | | |
| Inhibition of Very Long-Chain | Benzofurans | Benfuresate |
| Fatty Acid Synthesis | | |
| Inhibition of Very Long-Chain | Benzofurans | Ethofumesate |
| Fatty Acid Synthesis | | |
| Auxin Mimics | Pyridine-carboxylates | Picloram |
| Auxin Mimics | Pyridine-carboxylates | Clopyralid |
| Auxin Mimics | Pyridine-carboxylates | Aminopyralid |
| Auxin Mimics | Pyridine-carboxylates | Halauxifen |
| Auxin Mimics | Pyridine-carboxylates | Florpyrauxifen |
| Auxin Mimics | Pyridyloxy-carboxylates | Triclopyr |
| Auxin Mimics | Pyridyloxy-carboxylates | Fluroxypyr |
| Auxin Mimics | Phenoxy-carboxylates | 2,4,5-T |
| Auxin Mimics | Phenoxy-carboxylates | 2,4-D |
| Auxin Mimics | Phenoxy-carboxylates | 2,4-DB |
| Auxin Mimics | Phenoxy-carboxylates | Clomeprop |
| Auxin Mimics | Phenoxy-carboxylates | Dichlorprop |
| Auxin Mimics | Phenoxy-carboxylates | Fenoprop |
| Auxin Mimics | Phenoxy-carboxylates | Mecoprop |
| Auxin Mimics | Phenoxy-carboxylates | MCPA |
| Auxin Mimics | Phenoxy-carboxylates | MCPB |
| Auxin Mimics | Benzoates | Dicamba |
| Auxin Mimics | Benzoates | Chloramben |
| Auxin Mimics | Benzoates | TBA |
| Auxin Mimics | Quinoline-carboxylates | Quinclorac |
| Auxin Mimics | Quinoline-carboxylates | Quinmerac |
| Auxin Mimics | Pyrimidine-carboxylates | Aminocyclopyrachlor |
| Auxin Mimics | Other | Benazolin-ethyl |
| Auxin Mimics | Phenyl carboxylates | Chlorfenac = fenac |
| Auxin Mimics | Phenyl carboxylates | Chlorfenprop |
| Auxin Transport Inhibitor | Aryl-carboxylates | Naptalam |
| Auxin Transport Inhibitor | Aryl-carboxylates | Diflufenzopyr-sodium |
| Inhibition of Fatty Acid | Benzyl ether | Cinmethylin |
| Thioesterase | | |
| Inhibition of Fatty Acid | Benzyl ether | Methiozolin |
| Thioesterase | | |
| Inhibition of Serine-Threonine | Other | Endothal |
| Protein Phosphatase | | |
| Inhibition of Solanesyl | Diphenyl ether | Aclonifen |
| Diphosphate Synthase | | |
| Inhibition of Lycopene | Triazole | Amitrole |
| Cyclase | | |
| Unknown | | Bromobutide |
| Unknown | | Cumyluron |
| Unknown | | Difenzoquat |
| Unknown | | DSMA |
| Unknown | | Dymron = Daimuron |
| Unknown | | Etobenzanid |
| Unknown | Arylaminopropionic acid | Flamprop-m |
| Unknown | | Fosamine |
| Unknown | | Methyldymron |
| Unknown | | Monalide |
| Unknown | | MSMA |
| Unknown | | Oleic acid |
| Unknown | | Oxaziclomefone |
| Unknown | | Pelargonic acid |
| Unknown | | Pyributicarb |
| Unknown | | Quinoclamine |
| Unknown | Acetamides | Diphenamid |
| Unknown | Acetamides | Naproanilide |
| Unknown | Acetamides | Napropamide |
| Unknown | Benzamide | Tebutam |
| Unknown | Phosphorodithioate | Bensulide |
| Unknown | Chlorocarbonic acids | Dalapon |
| Unknown | Chlorocarbonic acids | Flupropanate |
| Unknown | Chlorocarbonic acids | TCA |
| Unknown | Trifluoromethanesulfonanilides | Mefluidide |
| Unknown | Trifluoromethanesulfonanilides | Perfluidone |
| Unknown | | CAMA |
| Unknown | | Cacodylic acid |

APPENDIX 3

| | Sub-group, class or | |
|---|---|---|
| Main Group and Primary Site of | exemplifying Active | |
| Action | Ingredient | Active Ingredients |
| 1 | 1A | Alanycarb, Aldicarb, Bendiocarb, Benfuracarb, |
| Acetylcholinesterase (AChE) | Carbamates | Butoxycarboxim, Carbaryl, Carbofuran, Carbosulfan, |
| inhibitors | | Ethiofencarb, Fenobucarb, Formetanate, Furathiocarb, |
| Nerve action | | Isoprocarb, Methiocarb, Methomyl, Metolcarb, Oxamyl, |
| {Strong evidence that action at this | | Pirimicarb, Propoxur, Thiodicarb, Thiofanox, |
| protein is responsible for insecticidal | | Triazamate, Trimethacarb, XMC, Xylylcarb |
| effects} | 1B | Acephate, Azamethiphos, Azinphos-ethyl, Azinphos- |
| | Organophosphates | methyl, Cadusafos, Chlorethoxyfos, Chlorfenvinphos, |
| | | Chlormephos, Chlorpyrifos, Chlorpyrifos-methyl, |
| | | Coumaphos, Cyanophos, Demeton-S-methyl, Diazinon, |
| | | Dichlorvos/DDVP, Dicrotophos, Dimethoate, |
| | | Dimethylvinphos, Disulfoton, EPN, Ethion, Ethoprophos, |
| | | Famphur, Fenamiphos, Fenitrothion, Fenthion, |
| | | Fosthiazate, Heptenophos, Imicyafos, Isofenphos, |
| | | Isopropyl O-(methoxyaminothio- phosphoryl) salicylate, |
| | | Isoxathion, Malathion, Mecarbam, Methamidophos, |
| | | Methidathion, Mevinphos, Monocrotophos, Naled, |
| | | Omethoate, Oxydemeton-methyl, Parathion, Parathion- |
| | | methyl, Phenthoate, Phorate, Phosalone, Phosmet, |
| | | Phosphamidon, Phoxim, Pirimiphos- methyl, Profenofos, |
| | | Propetamphos, Prothiofos, Pyraclofos, Pyridaphenthion, |
| | | Quinalphos, Sulfotep, Tebupirimfos, Temephos, Terbufos, |
| | | Tetrachlorvinphos, Thiometon, Triazophos, Trichlorfon, |
| | | Vamidothion |
| 2 | 2A | Chlordane, Endosulfan |
| GABA-gated chloride channel blockers | Cyclodiene | |
| Nerve action | Organochlorines | |
| {Strong evidence that action at this | 2B | Ethiprole, Fipronil |
| protein is responsible for insecticidal | Phenylpyrazoles (Fiproles) | |
| effects} | | |
| 3 | 3A | Acrinathrin, Allethrin, d-cis-trans Allethrin, d- trans Allethrin, |
| Sodium channel modulators | Pyrethroids Pyrethrins | Bifenthrin, Bioallethrin, Bioallethrin S-cyclopentenyl isomer, |
| Nerve action | | Bioresmethrin, Cycloprothrin, Cyfluthrin, beta- Cyfluthrin, |
| {Strong evidence that action at this | | Cyhalothrin, lambda-Cyhalothrin, gamma-Cyhalothrin, |
| protein is responsible for insecticidal | | Cypermethrin, alpha- Cypermethrin, beta-Cypermethrin, |
| effects} | | theta- cypermethrin, zeta-Cypermethrin, Cyphenothrin, |
| | | (1R)-trans- isomers], Deltamethrin, Empenthrin (EZ)- (1R)- |
| | | isomers], Esfenvalerate, Etofenprox, Fenpropathrin, |
| | | Fenvalerate, Flucythrinate, Flumethrin, tau-Fluvalinate, |
| | | Halfenprox, Imiprothrin, Kadethrin, Permethrin, Phenothrin |
| | | [(1R)-trans- isomer], Prallethrin, Pyrethrins (pyrethrum), |
| | | Resmethrin, Silafluofen, Tefluthrin, Tetramethrin, |
| | | Tetramethrin [(1R)-isomers], Tralomethrin, Transfluthrin, |
| | 3B | DDT |
| | DDT | Methoxychlor |
| | Methoxychlor | |
| 4 | 4A | Acetamiprid, Clothianidin, Dinotefuran, |
| Nicotinic acetylcholine receptor | Neonicotinoids | Imidacloprid, Nitenpyram, Thiacloprid, Thiamethoxam, |
| (nAChR) competitive modulators | 4B | Nicotine |
| Nerve action | Nicotine | |
| {Strong evidence that action at one or | 40 | Sulfoxaflor |
| more of this class of protein is | Sulfoximines | |
| responsible for insecticidal effects} | 4D | Flupyradifurone |
| | Butenolides | |
| | 4E | Triflumezopyrim |
| | Mesoionics | |
| | 4F | Flupyrimin |
| | Pyridylidenes | |
| 5 | Spinosyns | Spinetoram, Spinosad |
| Nicotinic acetylcholine receptor | | |
| (nAChR) allosteric modulators - Site I | | |
| Nerve action | | |
| {Strong evidence that action at one or | | |
| more of this class of protein is | | |
| responsible for insecticidal effects} | | |
| 6 | Avermectins | Abamectin, Emamectin benzoate, Lepimectin, Milbemectin |
| Glutamate-gated chloride | Milbemycins | |
| channel (GluCl) allosteric | | |
| modulators | | |
| Nerve and muscle action | | |
| {Strong evidence that action at one or | | |
| more of this class of protein is | | |
| responsible for insecticidal effects} | | |
| 7 | 7A | Hydroprene, Kinoprene, Methoprene |
| Juvenile hormone mimics | Juvenile hormone | |
| Growth regulation | analogues | |
| {Target protein responsible for biological | 7B | Fenoxycarb |
| activity is unknown, or uncharacterized} | Fenoxycarb | |
| | 7C | Pyriproxyfen |
| | Pyriproxyfen | |
| 8 * | 8A | Methyl bromide and other alkyl halides |
| Miscellaneous non-specific (multi- | Alkyl halides | |
| site) inhibitors | 8B | Chloropicrin |
| | Chloropicrin | |
| | 8C | Cryolite (Sodium aluminum fluoride), Sulfuryl fluoride |
| | Fluorides | |
| | 8D | Borax, Boric acid, Disodium octaborate, Sodium borate, |
| | Borates | Sodium metaborate |
| | 8E | Tartar emetic |
| | Tartar emetic | |
| | 8F | Dazomet, Metam |
| | Methyl isothiocyanate | |
| | generators | |
| 9 | 9B | Pymetrozine, Pyrifluquinazon |
| Chordotonal organ TRPV | Pyridine azomethine | |
| channel modulators Nerve action | derivatives | |
| {Strong evidence that action at one or | 9D | Afidopyropen |
| more of this class of proteins is | Pyropenes | |
| responsible for insecticidal effects} | | |
| 10 | 10A | Clofentezine, Diflovidazin, Hexythiazox |
| Mite growth inhibitors affecting | Clofentezine Diflovidazin | |
| CHS1 | Hexythiazox | |
| Growth regulation | 10B | Etoxazole |
| {Strong evidence that action at one or | Etoxazole | |
| more of this class of proteins is | | |
| responsible for insecticidal effects} | | |
| 11 | 11A | Bacillus thuringiensis subsp. israelensis Bacillus |
| Microbial disruptors of insect midgut | Bacillus thuringiensis and | thuringiensis subsp. aizawai Bacillus thuringiensis |
| membranes | the insecticidal proteins | subsp. kurstaki Bacillus thuringiensis subsp. |
| (Includes transgenic crops expressing | they produce | tenebrionis |
| Bacillus thuringiensis toxins, however | | B.t. crop proteins: (* Please see footnote) Cry1Ab, Cry1Ac, |
| specific guidance for resistance | | Cry1Fa, Cry1A.105, Cry2Ab, Vip3A, mCry3A, Cry3Ab, |
| management of transgenic crops is not | | Cry3Bb, Cry34Ab1/Cry35Ab1 |
| based on rotation of modes of action) | 11B | Bacillus sphaericus |
| | Bacillus sphaericus | |
| 12 | 12A | Diafenthiuron |
| Inhibitors of mitochondrial ATP | Diafenthiuron | |
| synthase | 12B | Azocyclotin, Cyhexatin, Fenbutatin oxide |
| Energy metabolism | Organotin miticides | |
| {Compounds affect the function of this | 12C | Propargite |
| protein, but it is not clear that this is what | Propargite | |
| leads to biological activity} | 12D | Tetradifon |
| | Tetradifon | |
| 13 * | Pyrroles Dinitrophenols | Chlorfenapyr DNOC |
| Uncouplers of oxidative | Sulfluramid | Sulfluramid |
| phosphorylation via disruption of | | |
| the proton gradient | | |
| Energy metabolism | | |
| 14 | Nereistoxin analogues | Bensultap, Cartap hydrochloride, Thiocyclam, |
| Nicotinic acetylcholine receptor | | Thiosultap-sodium |
| (nAChR) channel blockers | | |
| Nerve action | | |
| {Compounds affect the function of this | | |
| protein, but it is not clear that this is what | | |
| leads to biological activity} | | |
| 15 | Benzoylureas | Bistrifluron, Chlorfluazuron, Diflubenzuron, Flucycloxuron, |
| Inhibitors of chitin biosynthesis | | Flufenoxuron, Hexaflumuron, Lufenuron, Novaluron, |
| affecting CHS1 | | Noviflumuron, Teflubenzuron, Triflumuron |
| Growth regulation | | |
| {Strong evidence that action at one or | | |
| more of this class of proteins is | | |
| responsible for insecticidal effects} | | |
| 16 | Buprofezin | Buprofezin |
| Inhibitors of chitin biosynthesis, | | |
| type 1 | | |
| Growth regulation | | |
| {Target protein responsible for biological | | |
| activity is unknown, or uncharacterized} | | |
| 17 | Cyromazine | Cyromazine |
| Moulting disruptors, Dipteran | | |
| Growth regulation | | |
| {Target protein responsible for biological | | |
| activity is unknown, or uncharacterized} | | |
| 18 | Diacylhydrazines | Chromafenozide, Halofenozide, Methoxyfenozide, |
| Ecdysone receptor agonists | | Tebufenozide |
| Growth regulation | | |
| {Strong evidence that action at this | | |
| protein is responsible for insecticidal | | |
| effects} | | |
| 19 | Amitraz | Amitraz |
| Octopamine receptor agonists | | |
| Nerve action | | |
| {Good evidence that action at one or | | |
| more of this class of protein is | | |
| responsible for insecticidal effects} | | |
| 20 | 20A | Hydramethylnon |
| Mitochondrial complex III electron | Hydramethylnon | |
| transport inhibitors - Qo site | 20B | Acequinocyl |
| Energy metabolism | Acequinocyl | |
| {Good evidence that action at this | 20C | Fluacrypyrim |
| protein complex is responsible for | Fluacrypyrim | |
| insecticidal effects} | 20D | Bifenazate |
| | Bifenazate | |
| 21 | 21A | Fenazaquin, Fenpyroximate, Pyridaben, Pyrimidifen, |
| Mitochondrial complex I electron | METI acaricides and | Tebufenpyrad, Tolfenpyrad |
| transport inhibitors | insecticides | |
| Energy metabolism | 21B | Rotenone (Derris) |
| {Good evidence that action at this | Rotenone | |
| protein complex is responsible for | | |
| insecticidal effects} | | |
| 22 | 22A | Indoxacarb |
| Voltage-dependent sodium | Oxadiazines | |
| channel blockers | 22B | Metaflumizone |
| Nerve action | Semicarbazones | |
| {Good evidence that action at this | | |
| protein complex is responsible for | | |
| insecticidal effects} | | |
| 23 | Tetronic and Tetramic acid | Spirodiclofen, Spiromesifen, Spiropidion, Spirotetramat |
| Inhibitors of acetyl CoA carboxylase | derivatives | |
| Lipid synthesis, growth regulation | | |
| {Good evidence that action at this | | |
| protein is responsible for insecticidal | | |
| effects} | | |
| 24 | 24A | Aluminium phosphide, Calcium phosphide, Phosphine, Zinc |
| Mitochondrial complex IV electron | Phosphides | phosphide |
| transport inhibitors | 24B | Calcium cyanide, Potassium cyanide, Sodium cyanide |
| Energy metabolism | Cyanides | |
| {Good evidence that action at this | | |
| protein complex is responsible for | | |
| insecticidal effects} | | |
| 25 | 25A | Cyenopyrafen, Cyflumetofen |
| Mitochondrial complex II electron | Beta-ketonitrile | |
| transport inhibitors | derivatives | |
| Energy metabolism | 25B | Pyflubumide |
| {Good evidence that action at this | Carboxanilides | |
| protein complex is responsible for | | |
| insecticidal effects} | | |
| 28 | Diamides | Chlorantraniliprole, Cyantraniliprole, Cyclaniliprole |
| Ryanodine receptor | | Flubendiamide, Tetraniliprole |
| modulators | | |
| Nerve and muscle action | | |
| {Strong evidence that action at this | | |
| protein complex is responsible for | | |
| insecticidal effects} | | |
| 29 | Flonicamid | Flonicamid |
| Chordotonal organ modulators - | | |
| undefined target site | | |
| Nerve action | | |
| (Modulation of chordotonal organ | | |
| function has been clearly demonstrated, | | |
| but the specific target protein(s) | | |
| responsible for biological activity are | | |
| distinct from Group 9 and remain | | |
| undefined) | | |
| 30 | Meta-diamides Isoxazolines | Broflanilide |
| GABA-gated chloride channel allosteric | | Fluxametamide, Isocyloseram |
| modulators | | |
| Nerve action | | |
| {Strong evidence that action at this | | |
| protein complex is responsible for | | |
| insecticidal effects} | | |
| 31 | Granuloviruses | Cydia pomonella GV |
| Baculoviruses | (GVs) | Thaumatotibia leucotreta GV |
| Host-specific occluded | Nucleopolyhedroviruse s | Anticarsia gemmatalis MNPV |
| pathogenic viruses | (NPVs) | Helicoverpa armigera NPV |
| (Midgut epithelial columnar cell | | |
| membrane target site - undefined) | | |
| 32 | GS-omega/kappa | GS-omega/kappa HXTX-Hv1a peptide |
| Nicotinic Acetylcholine Receptor | HXTX-Hv1a peptide | |
| (nAChR) Allosteric Modulators - Site II | | |
| Nerve action | | |
| {Strong evidence that action at one or | | |
| more of this class of protein is | | |
| responsible for insecticidal effects} | | |
| 33 | Acynonapyr | Acynonapyr |
| Calcium-activated potassium | | |
| channel (KCa2) modulators | | |
| Nerve action | | |
| {Strong evidence that action at this | | |
| protein is responsible for insecticidal | | |
| effects} | | |
| 34 | Flometoquin | Flometoquin |
| Mitochondrial complex III electron | | |
| transport inhibitors - Qi site | | |
| Energy metabolism | | |
| {Modulation of this protein complex has | | |
| been clearly demonstrated and the specific | | |
| target site responsible for biological activity | | |
| is distinct from Group 20} | | |
| UN* | Azadirachtin | Azadirachtin |
| Compounds of unknown or uncertain | Benzoximate | Benzoximate |
| MoA | Benzpyrimoxan | Benzpyrimoxan |
| {Target protein responsible for biological | Bromopropylate | Bromopropylate |
| activity is unknown, or uncharacterized} | Chinomethionat | Chinomethionat |
| | Dicofol | Dicofol |
| | Lime sulfur | Lime sulfur |
| | Mancozeb | Mancozeb |
| | Pyridalyl | Pyridalyl |
| | Sulfur | Sulfur |
| UNB* | | Burkholderia spp |
| Bacterial agents (non-Bt) of unknown or | | Wolbachia pipientis (Zap) |
| uncertain MoA | | |
| {Target protein responsible for biological | | |
| activity is unknown or uncharacterized} | | |
| UNE* | | Chenopodium ambrosioides near ambrosioides |
| Botanical essence including | | extract |
| synthetic, extracts and unrefined oils | | Fatty acid monoesters with glycerol or propanediol Neem oil |
| with unknown or uncertain MoA | | |
| {Target protein responsible for biological | | |
| activity is unknown, or uncharacterized} | | |
| UNF* | | Beauveria bassiana strains |
| Fungal agents of unknown or uncertain | | Metarhizium anisopliae strain F52 |
| MoA | | Paecilomyces fumosoroseus Apopka strain 97 |
| {Target protein responsible for biological | | |
| activity is unknown, or uncharacterized} | | |
| UNM* | | Diatomaceous earth |
| Non-specific mechanical and physical | | Mineral oil |
| disruptors | | |
| {Target protein responsible for biological | | |
| activity is unknown, or uncharacterized} | | |

1. A method for inhibiting apyrase, comprising contacting the apyrase with a compound of the formula 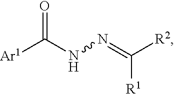
wherein Ar¹ is selected from aryl and heteroaryl;
R¹ is selected from hydrogen, C₃₋₆ cycloalkyl, C₁₋₆ alkyl, aralkyl, and C₁₋₃ haloalkyl;
R² is selected from alkyl, aryl and heteroaryl; provided that the compound does not have the formula

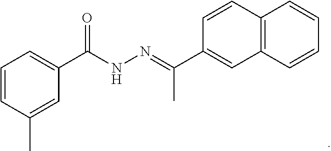 [SMILES: C/C(=N\NC(=O)c1cccc(C)c1)c1ccc2ccccc2c1]

2. The method of claim 1, wherein the compound has the formula 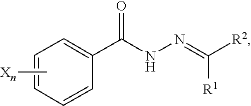
wherein X is, for each occurrence, independently selected from R^{a}, R^{b}, R^{a} substituted with one or more of the same or different R^{b}, —OR^{a} substituted with one or more of the same or different R^{b} or R^{d}, or
(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂,
O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b},
—C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b},
O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b},
—NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b};
or two X substituents together with the atoms to which they are attached form a 5 to 8-membered aryl, cycloalkyl, heterocyclylalkyl or heteroaryl which may optionally include one or more of the same or different additional heteroatoms and which may optionally be substituted with one or more of the same or different R^{b} groups;
each R^{a} is independently selected from the group consisting of C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl, 3-8 membered heterocyclylalkyl, 4-11 membered heterocyclylalkyl alkyl, 5-10 membered heteroaryl and 6-16 membered heteroarylalkyl;
R^{b} is a group independently selected from the group consisting of ═O, —OR^{d}, C₁₋₃ haloalkyloxy, —OCF₂H, —OCH₂F, —OCF₃, ═S, —SR^{d}, —SCF₃, —SF₅, ═NR^{d}, ═NOR^{d}, —NR^{c}R^{c},
halogen, —CF₃, —CN, —NO₂, —S(O)R^{d}, —S(O)₂R^{d}, —S(O)₂CF₃, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
—OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[N R^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c} is independently R^{a}, or, alternatively, two R^{c} are taken together with the nitrogen atom to which they are bonded to form a 5 to 8-membered heterocyclylalkyl or heteroaryl which may optionally include one or more of the same or different additional heteroatoms and which may optionally be substituted with one or more of the same or different R^{b} groups;
each R^{d} is independently hydrogen or C₁₋₆ alkyl;
each m is independently an integer from 1 to 3; and
each n is independently an integer from 0 to 3.

3. The method of claim 2, wherein X is selected from C₁₋₆ alkyl, —OR^{a}, —S(O)₂NR^{c}R^{c} and halogen.

4. The method of claim 1, wherein Ar¹ is heteroaryl.

5. The method of claim 1, wherein Ar¹ is monocyclic heteroaryl.

6. The method of claim 1, wherein Ar¹ is bicyclic heteroaryl.

7. The method of claim 1, wherein the compound has the formula 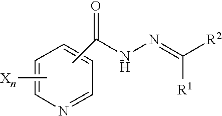
wherein X is, for each occurrence, independently selected from R^{a}, R^{b}, R^{a} substituted with one or more of the same or different R^{b}, —OR^{a} substituted with one or more of the same or different R^{b} or R^{d}, or
—(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂,
—O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b},
—C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b},
—O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b},
—NH—(CHR^{a})ₘ—R^{b}, —NH[(CH₂)ₘR^{b}], —N[(CH₂)ₘR]₂, —NH—C(O)—NH—(CH₂)ₘ—R^{b}, —NH—C(O)—(CH₂)ₘ—CHR^{b}R^{b};
or two X substituents together with the atoms to which they are attached to form a 5 to 8-membered aryl, cycloalkyl, heterocyclylalkyl or heteroaryl which may optionally include one or more of the same or different additional heteroatoms and which may optionally be substituted with one or more of the same or different R^{b} groups;
each R^{a} is independently selected from the group consisting of C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl, 3-8 membered heterocyclylalkyl, 4-11 membered heterocyclylalkyl alkyl, 5-10 membered heteroaryl and 6-16 membered heteroarylalkyl;
R^{b} is a group independently selected from the group consisting of ═O, —OR a, C₁₋₃ haloalkyloxy,-OCF₂H, —OCH₂F, —OCF₃, ═S, —SR^{a}, —SCF₃, —SF₅, ═NR^{d}, ═NOR^{c}R^{c}, —NR^{c}R^{c},
halogen, —CF₃, —CN, —NO₂, —S(O)R^{d}, —S(O)₂R^{d}, —S(O)₂CF₃, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
—OC(O)NR^{c}W, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[N R^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c} is independently R^{a}, or, alternatively, two R^{c} are taken together with the nitrogen atom to which they are bonded to form a 5 to 8-membered heterocyclylalkyl or heteroaryl which may optionally include one or more of the same or different additional heteroatoms and which may optionally be substituted with one or more of the same or different R^{b} groups;
each R^{d} is independently hydrogen or C₁₋₆ alkyl;
each m is independently an integer from 1 to 3; and
each n is independently an integer from 0 to 3.

8. The method of claim 1, wherein the compound has the formula

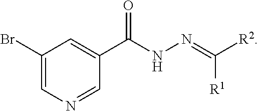

9. The method of claim 1, wherein the compound has the formula

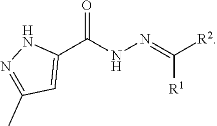

10. The method of claim 1, wherein the compound has the formula

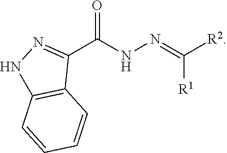

11. The method of claim 1, wherein the compound has the formula

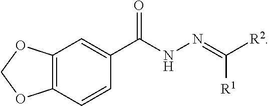

12. The method of claim 1, wherein the compound has the formula

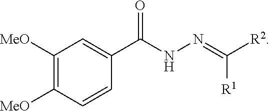

13. The method of claim 1, wherein R² is hydrogen.

14. The method of claim 1, wherein R² is methyl.

15. The method of claim 1, wherein R² is heteroaryl.

16. The method of claim 1, wherein R² is alkyl.

17. The method of claim 1, wherein R² is aryl.

18. The method of claim 1, wherein the compound has the formula 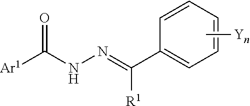
wherein Y is, for each occurrence, independently selected from R^{a}, R^{b}, R^{a} substituted with one or more of the same or different R^{b}, —OR^{a} substituted with one or more of the same or different R^{b} or R^{d}, or
—(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂,
—O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b},
—C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b},
—O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b},
—NH—(CHR^{a})ₘ—R^{b}, —NH[(CH₂)ₘR^{b}], —N[(CH₂)ₘR]₂, —NH—C(O)—NH—(CH₂)ₘ—R^{b}, —NH—C(O)—(CH₂)ₘ—CHR^{b}R^{b};
or two Y substituents together with the atoms to which they are attached to form a 5 to 8-membered aryl, cycloalkyl, heterocyclylalkyl or heteroaryl which may optionally include one or more of the same or different additional heteroatoms and which may optionally be substituted with one or more of the same or different include alkyl, cycloalkyl, and R^{b} groups;
each R^{a} is independently selected from the group consisting of C₁₋₆ alkyl, C₃₋₈ cycloalkyl, C₆₋₁₀ aryl, C₅₋₁₀ heteroaryl, C₆₋₁₆ arylalkyl, 2-6 membered heteroalkyl, 3-8 membered heterocyclylalkyl, 4-11 membered heterocyclylalkyl alkyl, 5-10 membered heteroaryl and 6-16 membered heteroarylalkyl;
R^{b} is a group independently selected from the group consisting of ═O, —OR^{d}, C₁-3 haloalkyloxy,-OCF₂H, —OCH₂F, —OCF₃, ═S, —SR^{d}, —SCF₃, —SF₅, ═NR^{d}, ═NOR^{d}, —NR^{c}R^{c}, halogen, —CF₃, —CN, —NO₂, —S(O)R^{d}, —S(O)₂R^{d}, —S(O)₂CF₃, —S(O)₂OR^{d}, —S(O)NR^{c}R^{c}, —S(O)₂NR^{c}R^{c},
—OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d},
—OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[N R^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c} is independently R^{a}, or, alternatively, two R^{c} are taken together with the nitrogen atom to which they are bonded to form a 5 to 8-membered heterocyclylalkyl or heteroaryl which may optionally include one or more of the same or different additional heteroatoms and which may optionally be substituted with one or more of the same or different R^{b} groups;
each R^{d} is independently hydrogen or C₁₋₆ alkyl;
each m is independently an integer from 1 to 3; and
each n is independently an integer from 0 to 3.

19. The method of claim 1, wherein the compound has the formula

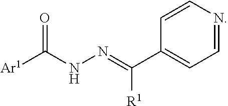

20. The method of claim 19, wherein Ar¹ is optionally substituted phenyl.

21. The method of claim 1, wherein the compound is selected from
(E)-N-(2-chlorophenyl)-3-(2-(1-(naphthalen-2-yl)ethylidene)hydrazine-1-carbonyl)benzenesulfonamide;
(E)-N-(4-(2-(1-(naphthalen-2-yl)ethylidene)hydrazine-1-carbonyl)phenyl)thiophene-2-sulfonamide;
(E)-3-(morpholinosulfonyl)-N′-(1-(naphthalen-2-yl)ethylidene)benzohydrazide;
(E)-3-fluoro-N′-(1-(pyridin-2-yl)ethylidene)benzohydrazide;
(Z)-N′-(undecan-2-ylidene)benzohydrazide;
(E)-5-bromo-N′-(1-(naphthalen-2-yl)ethylidene)nicotinohydrazide;
(Z)-3-methyl-N′-(1-(naphthalen-2-yl)ethylidene)benzohydrazide;
(E)-N′-(1-(naphthalen-2-yl)ethylidene)-4-(1H-tetrazol-1-yl)benzohydrazide;
(E)-N′-pentylidenebenzohydrazide;
(E)-N′-(1-(pyridin-3-yl)ethylidene)benzo[d][1,3]dioxole-5-carbohydrazide;
(E)-N′-(1-(3,4-dimethylphenyl)ethylidene)-1-naphthohydrazide;
(E)-2-fluoro-N′-(1-(pyridin-4-yl)ethylidene)benzohydrazide;
(E)-N′-(1-(6-methoxynaphthalen-2-yl)ethylidene)benzo[d][1,3]dioxole-5-carbohydrazide;
(E)-N′-(1-(naphthalen-2-yl)ethylidene)-4,5,6,7-tetrahydro-1H-indazole-3-carbohydrazide;
(E)-3-methyl-N′-(1-(naphthalen-2-yl)ethylidene)-1H-pyrazole-5-carbohydrazide;
(E)-N′-(1-(naphthalen-1-yl)ethylidene)hexanohydrazide;
(Z)-2-(2-benzoylhydrazineylidene)propanoic acid;
(E)-2-methyl-N′-(1-(pyridin-2-yl)ethylidene)benzohydrazide;
(E)-N′-butylidenebenzohydrazide;
(E)-3,4-dimethoxy-N′-(1-(6-methoxynaphthalen-2-yl)ethylidene)benzohydrazide;
(Z)-N′-(1-(naphthalen-2-yl)ethylidene)-1H-benzo[d]imidazole-6-carbohydrazide;
(E)-N′-(1-([1,1′-biphenyl]-4-yl)ethylidene)benzohydrazide;
(E)-2-(1-(naphthalen-1-yl)ethylidene)hydrazine-1-carboxamide;
(Z)-N′-(1-(naphthalen-2-yl)ethylidene)hexanohydrazide;
(E)-N′-(4-ethylbenzylidene)benzohydrazide;
(E)-N′-(2,2-dimethylpropylidene)benzohydrazide;
(E)-2,4-dichloro-N′-(1-(pyridin-2-yl)ethylidene)benzohydrazide;
(E)-3,4-dimethoxy-N′-(1-(pyridin-2-yl)ethylidene)benzohydrazide;
(E)-N′-(1-(naphthalen-2-yl)ethylidene)-3-nitrobenzohydrazide;
2-fluoro-N′-(heptan-4-ylidene)benzohydrazide;
(E)-3-chloro-N′-(1-(naphthalen-2-yl)ethylidene)benzohydrazide;
(Z)-N′-(1-cyclopropylethylidene)-3-methylbenzohydrazide;
(E)-N′-(4-methylbenzylidene)benzohydrazide;
N′-(4-(tert-butyl)cyclohexylidene)benzohydrazide;
(E)-N′-(1-(2,4-dimethylphenyl)ethylidene)-3,4-dimethylbenzohydrazide;
N′-(propan-2-ylidene)-1-naphthohydrazide;
(E)-4-methyl-N′-(1-phenylethylidene)benzohydrazide;
(E)-3-nitro-N′-(1-(pyridin-2-yl)ethylidene)benzohydrazide;
(E)-3-methyl-4-nitro-N′-(1-(pyridin-2-yl)ethylidene)benzohydrazide;
(E)-N′-(1-(2-chlorophenyl)ethylidene)-1-naphthohydrazide;
(E)-3-bromo-N′-(1-(pyridin-2-yl)ethylidene)benzohydrazide;
(E)-3-methoxy-N′-(1-(pyridin-2-yl)ethylidene)benzohydrazide;
(E)-4-methyl-N′-(1-(pyridin-4-yl)ethylidene)benzohydrazide;
(E)-2,5-dichloro-N′-(1-(pyridin-2-yl)ethylidene)benzohydrazide;
(E)-2,3-dichloro-N′-(1-(pyridin-2-yl)ethylidene)benzohydrazide;
(E)-4-(2-(1-(2,4-dimethylphenyl)ethylidene)hydrazine-1-carbonyl)benzamide;
(E)-3-bromo-N′-(1-(pyridin-3-yl)ethylidene)benzohydrazide;
(E)-2-hydroxy-2-methyl-N′-(naphthalen-2-ylmethylene)propanehydrazide;
(E)-3-methyl-N′-(1-(pyridin-2-yl)ethylidene)benzohydrazide;
(E)-3-chloro-N′-(1-(pyridin-2-yl)ethylidene)benzohydrazide;
(E)-3-methoxy-N′-(1-(pyridin-4-yl)ethylidene)benzohydrazide;
(E)-4-fluoro-N′-(1-(pyridin-3-yl)ethylidene)benzohydrazide;
(E)-4-hydroxy-N′-(1-(naphthalen-2-yl)ethylidene)benzohydrazide;
(E)-3-methoxy-N′-(1-(naphthalen-2-yl)ethylidene)benzohydrazide;
(E)-5-chloro-2-methoxy-N′-(1-(naphthalen-2-yl)ethylidene)benzohydrazide;
(E)-2-fluoro-N′-(1-(pyridin-2-yl)ethylidene)benzohydrazide;
(E)-3-nitro-N′-(1-(pyridin-3-yl)ethylidene)benzohydrazide;
(E)-N′-(1-(pyridin-2-yl)ethylidene)benzo[d][1,3]dioxole-5-carbohydrazide;
(E)-4-nitro-N′-(1-(pyridin-3-yl)ethylidene)benzohydrazide;
(E)-2-methoxy-N′-(1-(pyridin-3-yl)ethylidene)benzohydrazide;
(Z)-2-methoxy-N′-(1-(pyridin-4-yl)ethylidene)benzohydrazide;
(E)-2-methoxy-N′-(1-(pyridin-2-yl)ethylidene)benzohydrazide;
(E)-4-(dimethylamino)-N′-(1-(pyridin-2-yl)ethylidene)benzohydrazide;
(E)-N′-(1-(pyridin-3-yl)ethylidene)-1-naphthohydrazide;
(E)-N′-(1-(pyridin-2-yl)ethylidene)-1-naphthohydrazide;
(E)-N′-(1-(4-fluorophenyl)ethylidene)-1-naphthohydrazide;
(E)-N′-(1-(pyridin-4-yl)ethylidene)-1-naphthohydrazide;
(E)-N′-(1-(pyridin-3-yl)ethylidene)-2-naphthohydrazide;
(E)-N′-(1-(pyridin-4-yl)ethylidene)benzo[d][1,3]dioxole-5-carbohydrazide;
(E)-4-chloro-N′-(1-(pyridin-2-yl)ethylidene)benzohydrazide;
(E)-3-chloro-N′-(1-(pyridin-3-yl)ethylidene)benzohydrazide;
(E)-2-fluoro-N′-(1-(pyridin-3-yl)ethylidene)benzohydrazide;
(E)-3-bromo-N′-(1-(pyridin-4-yl)ethylidene)benzohydrazide;
(E)-4-bromo-1-methyl-N′-(1-(naphthalen-2-yl)ethylidene)-1H-pyrazole-3-carbohydrazide;
(E)-3,4,5-trimethoxy-N′-(1-(pyridin-4-yl)ethylidene)benzohydrazide;
(E)-4-methoxy-N′-(1-(pyridin-2-yl)ethylidene)benzohydrazide;
(E)-N′-(1-(naphthalen-2-yl)ethylidene)-2-nitrobenzohydrazide;
(E)-4-(dimethylamino)-N′-(1-(pyridin-4-yl)ethylidene)benzohydrazide;
(E)-4-(dimethylamino)-N′-(1-(pyridin-3-yl)ethylidene)benzohydrazide;
(E)-2-fluoro-N′-(1-(naphthalen-2-yl)ethylidene)benzohydrazide;
(E)-N′-(1-phenylethylidene)isobutyrohydrazide;
N′-cyclohexylidenebenzohydrazide;
(E)-N′-(1-phenylethylidene)propionohydrazide;
(Z)-N′-(phenyl(pyridin-2-yl)methylene)benzohydrazide;
(E)-2-methyl-N′-(2-methylpropylidene)benzohydrazide;
(E)-N′-butylidene-2-methylbenzohydrazide;
(E)-2-chloro-N′-(1-(naphthalen-2-yl)ethylidene)benzohydrazide;
(E)-2-hydroxy-N′-(naphthalen-1-ylmethylene)acetohydrazide;
(E)-2-methyl-N′-(1-(naphthalen-2-yl)ethylidene)furan-3-carbohydrazide;
(E)-N′-(1-(2-fluorophenyl)ethylidene)benzohydrazide;
(E)-N′-(1-(6-methoxynaphthalen-2-yl)ethylidene)-2,4-dimethylbenzohydrazide;
4-fluoro-N′-(heptan-4-ylidene)benzohydrazide;
(E)-N′-(1-(pyridin-2-yl)ethylidene)benzo[d][1,3]dioxole-5-carbohydrazide;
(E)-3,5-dimethoxy-N′-(1-(naphthalen-2-yl)ethylidene)benzohydrazide;
(E)-N′-(1-(6-methoxynaphthalen-2-yl)ethylidene)-2-(1H-pyrrol-1-yl)benzohydrazide;
(E)-N′-(1-(pyridin-3-yl)ethylidene)benzohydrazide;
(E)-4-(((4-methyl-4H-1,2,4-triazol-3-yl)thio)methyl)-N′-(1-(naphthalen-2-yl)ethylidene)benzohydrazide;
(E)-N′-(3,3,5-trimethylcyclohexylidene)benzohydrazide;
(E)-N′-(1-(1-(difluoromethoxy)naphthalen-2-yl)ethylidene)-2,4-dimethylbenzohydrazide;
(E)-4-(diethylamino)-N′-(1-(pyridin-3-yl)ethylidene)benzohydrazide;
(E)-3-nitro-N′-(1-(pyridin-4-yl)ethylidene)benzohydrazide;
(E)-N′-(3-methylcyclohexylidene)benzohydrazide;
(E)-4-hydroxy-N′-(1-phenylethylidene)benzohydrazide;
(E)-N′-(1-(3,4-dichlorophenyl)ethylidene)-1-naphthohydrazide;
(Z)-N′-(1-cyclopropylethylidene)benzohydrazide;
(E)-3-methyl-N′-(1-phenylpropylidene)benzohydrazide;
(E)-3-methyl-N′-(1-phenylpentylidene)benzohydrazide;
(Z)-N′-(1-phenylethylidene)acetohydrazide;
(E)-N′-(phenyl(pyridin-4-yl)methylene)benzohydrazide;
(E)-4-fluoro-N′-(1-(naphthalen-2-yl)ethylidene)benzohydrazide;
(E)-N′-(1-(naphthalen-2-yl)ethylidene)isonicotinohydrazide;
(Z)-4-fluoro-N′-(1-(pyridin-2-yl)ethylidene)benzohydrazide;
(Z)-N′-(1-(4-chlorophenyl)ethylidene)benzohydrazide;
(Z)-N′-(1-(2-chlorophenyl)ethylidene)benzohydrazide;
(E)-N′-(1-(naphthalen-2-yl)ethylidene)picolinohydrazide;
(Z)-N′-(1-(4-fluorophenyl)ethylidene)benzohydrazide;
(E)-N′-(2-chlorobenzylidene)-2-naphthohydrazide;
4-methyl-N′-(4-methylcyclohexylidene)benzohydrazide;
(E)-3-cyclopropyl-N′-(1-(naphthalen-2-yl)ethylidene)-1H-pyrazole-5-carbohydrazide;
(E)-N′-octylidenebenzohydrazide;
(E)-N′-(naphthalen-1-ylmethylene)acetohydrazide;
(E)-2-(2-benzoylhydrazineylidene)propanoic acid;
(E)-N′-benzylidene-4-methylbenzohydrazide;
(E)-4-chloro-N′-ethylidenebenzohydrazide;
(E)-N′-(1-phenylethylidene)acetohydrazide;
(E)-N′-(3-methylbenzylidene)benzohydrazide;
(E)-3-methyl-N′-(4-methylpentan-2-ylidene)benzohydrazide;
(E)-N′-(1-(naphthalen-2-yl)ethylidene)-[1,1′-biphenyl]-4-carbohydrazide;
(Z)-N′-(1-phenylbutylidene)benzohydrazide;
(Z)-3-methyl-N′-(1-phenylpentylidene)benzohydrazide;
N′-cyclohexylidene-3-methylbenzohydrazide;
(E)-N′-(1-phenylethylidene)benzohydrazide;
N′-cyclopentylidene-2-methylbenzohydrazide;
(E)-3-bromo-N′-(1-(naphthalen-2-yl)ethylidene)benzohydrazide;
(E)-N′-(naphthalen-2-ylmethylene)benzohydrazide;
(E)-3-methyl-N′-(naphthalen-2-ylmethylene)benzohydrazide;
(E)-3-methyl-N′-(2-methylbenzylidene)benzohydrazide;
(E)-4-fluoro-N′-(1-phenylethylidene)benzohydrazide;
(E)-4-chloro-N′-(1-phenylethylidene)benzohydrazide;
(E)-4-chloro-N′-(1-(p-tolyl)ethylidene)benzohydrazide;
(E)-4-chloro-N′-(1-(4-chlorophenyl)ethylidene)benzohydrazide;
(E)-2-chloro-4-methyl-N′-(1-(naphthalen-2-yl)ethylidene)benzohydrazide;
(Z)-N′-(1-(naphthalen-2-yl)ethylidene)pyrazine-2-carbohydrazide;
(E)-2-methyl-N′-(1-(p-tolyl)ethylidene)benzohydrazide;
(Z)-3,4-dimethyl-N′-(1-(pyridin-2-yl)ethylidene)benzohydrazide;
N′-(diphenylmethylene)isobutyrohydrazide;
4-amino-N′-cyclopentylidenebenzohydrazide;
(E)-4-(tert-butyl)-N′-(1-(naphthalen-2-yl)ethylidene)benzohydrazide;
(E)-N′-(1-(p-tolyl)ethylidene)benzohydrazide;
(E)-3-methyl-N′-(3-methylbenzylidene)benzohydrazide;
(E)-2,5-dimethyl-N′-(1-(naphthalen-2-yl)ethylidene)furan-3-carbohydrazide;
(E)-N′-(1-(naphthalen-2-yl)ethylidene)nicotinohydrazide;
(E)-N′-(1-(4-aminophenyl)ethylidene)benzohydrazide;
(E)-4,6-dimethyl-N′-(1-phenylethylidene)pyrimidine-2-carbohydrazide;
(E)-3-fluoro-N′-(1-(pyridin-4-yl)ethylidene)benzohydrazide;
(E)-N′-(2-methylpropylidene)benzohydrazide;
(E)-N′-(1-(3,4-dimethylphenyl)ethylidene)-3,4-dimethylbenzohydrazide;
(E)-3-methyl-N′-(1-(pyridin-4-yl)ethylidene)benzohydrazide;
(E)-2-methyl-N′-(1-(pyridin-4-yl)ethylidene)benzohydrazide;
(E)-N′-(4-cyanobenzylidene)-3-methylbenzohydrazide;
(E)-N′-(1-([1,1′-biphenyl]-4-yl)ethylidene)-3-methylbenzohydrazide;
(E)-3-methyl-N′-(1-phenylbutylidene)benzohydrazide;
(E)-N-(4-(2-(1-(naphthalen-2-yl)ethylidene)hydrazine-1-carbonyl)phenyl)propionamide;
(E)-N′-(1-(4-chlorophenyl)ethylidene)benzohydrazide;
(E)-4-(tert-butyl)-N′-(1-(p-tolyl)ethylidene)benzohydrazide;
(E)-N′-(1-(naphthalen-2-yl)ethylidene)cyclohexanecarbohydrazide;
(E)-N′-(1-(naphthalen-2-yl)ethylidene)cyclopropanecarbohydrazide;
(E)-N′-(1-(naphthalen-2-yl)ethylidene)-3-phenylpropanehydrazide;
N′-cyclopentylidene-3-methylbenzohydrazide;
(E)-4-chloro-N′-(1-(naphthalen-2-yl)ethylidene)benzohydrazide;
(E)-3,4-dimethyl-N′-(1-(pyridin-3-yl)ethylidene)benzohydrazide;
(E)-N′-(4-bromobenzylidene)-3-methylbenzohydrazide;
(E)-N′-(naphthalen-2-ylmethylene)-2-phenylacetohydrazide;
(Z)-3-fluoro-N′-(1-(naphthalen-2-yl)ethylidene)benzohydrazide;
methyl (E)-2-(1-(naphthalen-2-yl)ethylidene)hydrazine-1-carboxylate;
(E)-N′-benzylidenebenzohydrazide;
(E)-N′-(4-methylpent-3-en-2-ylidene)benzohydrazide;
5-(naphthalen-2-yl)-4H-pyrazol-3-ol;
(E)-2-methyl-N′-(3-methylbutan-2-ylidene)benzohydrazide;
(Z)-N′-(1-(naphthalen-2-yl)ethylidene)furan-2-carbohydrazide;
(E)-4-(tert-butyl)-N′-(1-(4-ethylphenyl)ethylidene)benzohydrazide;
(E)-2-chloro-N′-(1-phenylethylidene)benzohydrazide;
(E)-2-chloro-N′-(1-(pyridin-4-yl)ethylidene)benzohydrazide;
(E)-3,4-dichloro-N′-(1-(pyridin-2-yl)ethylidene)benzohydrazide;
(E)-N′-(1-(naphthalen-2-yl)ethylidene)-2-(thiophen-2-yl)acetohydrazide;
(Z)-3-cyclopropyl-N′-(1-(naphthalen-2-yl)ethylidene)-1H-pyrazole-5-carbohydrazide;
(E)-N′-(1-(naphthalen-2-yl)ethylidene)thiophene-2-carbohydrazide;
(Z)-N′-(1-(naphthalen-2-yl)ethylidene)nicotinohydrazide;
(Z)-N′-(1-(naphthalen-2-yl)ethylidene)picolinohydrazide;
(E)-N′-ethylidene-2-methylbenzohydrazide;
(Z)-3,4,5-trimethoxy-N′-(1-(pyridin-3-yl)ethylidene)benzohydrazide;
(E)-4-methyl-N′-(3-methylbutylidene)benzohydrazide;
(E)-N′-(1-cyclopropylethylidene)-3-methylbenzohydrazide;
(Z)-N′-(1-(naphthalen-2-yl)ethylidene)-3-phenylpropanehydrazide;
(E)-N′-(1-(naphthalen-2-yl)ethylidene)hexanohydrazide;
(E)-4-bromo-N′-(butan-2-ylidene)benzohydrazide;
(Z)-4-(dimethylamino)-N′-(1-(pyridin-4-yl)ethylidene)benzohydrazide;
(E)-3,4-dimethyl-N′-(1-(pyridin-2-yl)ethylidene)benzohydrazide;
(E)-N′-(3,3-dimethylbutan-2-ylidene)-4-methylbenzohydrazide;
(E)-4-(tert-butyl)-N′-(1-(pyridin-3-yl)ethylidene)benzohydrazide;
(E)-3-fluoro-N′-(1-(naphthalen-2-yl)ethylidene)benzohydrazide;
(E)-N′-(1-(naphthalen-2-yl)ethylidene)benzohydrazide;
(E)-3-methyl-N′-(1-(5,6,7,8-tetrahydronaphthalen-2-yl)ethylidene)benzohydrazide;
(E)-6-methyl-N′-(1-(naphthalen-2-yl)ethylidene)pyridazine-4-carbohydrazide;
(E)-3,4-dimethyl-N′-(1-(naphthalen-2-yl)ethylidene)benzohydrazide;
(E)-4-methyl-N′-(1-(naphthalen-2-yl)ethylidene)thiophene-2-carbohydrazide;
(E)-N-(4-(2-(1-(naphthalen-2-yl)ethylidene)hydrazine-1-carbonyl)phenyl)methanesulfonamide;
(E)-3-(dimethylamino)-N′-(1-(naphthalen-2-yl)ethylidene)benzohydrazide;
(E)-N′-(1-(naphthalen-2-yl)ethylidene)-3-(trifluoromethyl)benzohydrazide;
(E)-6-methyl-N′-(1-(naphthalen-2-yl)ethylidene)picolinohydrazide;
(E)-5-methyl-N′-(1-(naphthalen-2-yl)ethylidene)thiophene-2-carbohydrazide;
(E)-N′-(1-(naphthalen-2-yl)ethylidene)benzohydrazide;
(E)-N-(3-(2-(1-(naphthalen-2-yl)ethylidene)hydrazine-1-carbonyl)phenyl)methanesulfonamide;
(E)-3-methyl-N′-(1-(quinolin-3-yl)ethylidene)benzohydrazide;
(E)-5-methyl-N′-(1-(naphthalen-2-yl)ethylidene)thiophene-3-carbohydrazide;
(E)-N′-(1-(3,4-dimethylphenyl)ethylidene)-3-methylbenzohydrazide;
(E)-3-ethyl-N′-(1-(naphthalen-2-yl)ethylidene)benzohydrazide;
(E)-2-methyl-N′-(1-(naphthalen-2-yl)ethylidene)pyrimidine-4-carbohydrazide;
(E)-3-(hydroxymethyl)-N′-(1-(naphthalen-2-yl)ethylidene)benzohydrazide;
(E)-N,N-dimethyl-3-(2-(1-(naphthalen-2-yl)ethylidene)hydrazine-1-carbonyl)benzenesulfonamide;
(E)-N-methyl-3-(2-(1-(naphthalen-2-yl)ethylidene)hydrazine-1-carbonyl)benzenesulfonamide;
(E)-6-methyl-N′-(1-(naphthalen-2-yl)ethylidene)pyrimidine-4-carbohydrazide;
(E)-N′-(1-(2,3-dihydro-1H-inden-5-yl)ethylidene)-3-methylbenzohydrazide;
(E)-4-methyl-N′-(1-(naphthalen-2-yl)ethylidene)pyrimidine-2-carbohydrazide;
(E)-5-methyl-N′-(1-(naphthalen-2-yl)ethylidene)nicotinohydrazide;
(E)-4-methyl-N′-(1-(naphthalen-2-yl)ethylidene)picolinohydrazide;
(E)-N′-(1-(benzo[d][1,3]dioxol-5-yl)ethylidene)-3-methylbenzohydrazide;
(E)-2-methyl-N′-(1-(naphthalen-2-yl)ethylidene)isonicotinohydrazide;
(E)-3-methyl-N′-(1-(quinolin-2-yl)ethylidene)benzohydrazide;
(E)-N′-(1-(isoquinolin-3-yl)ethylidene)-3-methylbenzohydrazide;
(E)-3-methyl-N′-(1-phenylethylidene)benzohydrazide;
(E)-6-methyl-N′-(1-(naphthalen-2-yl)ethylidene)pyrazine-2-carbohydrazide;
(E)-3-(methylsulfonyl)-N′-(1-(naphthalen-2-yl)ethylidene)benzohydrazide; and
(E)-N′-(1-(2,3-dihydrobenzo[b][1,4]dioxin-6-yl)ethylidene)-3-methylbenzohydrazide.

22. The method of claim 1, wherein contacting the apyrase comprises treating a crop with the compound.

23. The method of claim 22, further comprising treating the crop with a pesticide.

24. The method of claim 23, wherein the pesticide is selected from acaricides, fungicides, herbicides, insecticides, molluscicides, nematocides, or a combination thereof.

25. The method of claim 24, wherein the pesticide comprises a fungicide.

26. The method of claim 22, further comprising treating the crop with a fungicide selected from selected from benzimidazoles, dicarboximides, phenylpyrroles, anilinopyrimidines, hydroxyanilides, carboxamides, phenyl amides, phosphonates, cinnamic acids, oxysterol binding protein inhibitors (OSBPI), triazole carboxamides, cymoxanil, carbamates, benzamides, demethylation inhibiting piperazines, demethylation inhibiting pyrimidines, demethylation inhibiting azoles, including imidazoles and triazoles, such as cyproconazole, difenoconazole, fenbuconazole, flutriafol, mefentrifluconazole, metconazole, ipconazole, prothioconazole, tebuconazole, tetraconazole, triadimefon, triadimenol, triticonazole, morpholines, cyflufenamid, metrafenone, pyriofenone, strobilurins, copper ammonium complex, copper hydroxide, copper oxide, copper oxychloride, copper sulfate, sulfur, lime sulfur, ethylenebisdithiocarbamates, aromatic hydrocarbons, phthalimides, guanidines, polyoxins, fluazinam, thiazolidines or a combination thereof.

27. A composition, comprising a compound of the formula 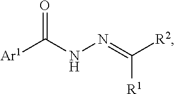
wherein Ar¹ is selected from aryl and heteroaryl;
R¹ is selected from hydrogen, C₃₋₆ cycloalkyl, C₁-6 alkyl, aralkyl, and C₁₋₃ haloalkyl;
R² is selected from alkyl, aryl and heteroaryl; provided that the compound does not have the formula 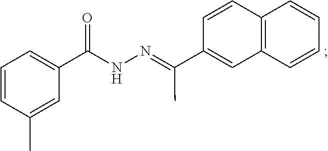 [SMILES: C/C(=N\NC(=O)c1cccc(C)c1)c1ccc2ccccc2c1]
and
a phytologically acceptable carrier.

28. The composition of claim 27, wherein the composition comprises from about 1 to about 80 weight percent of the compound.

29. The composition of claim 27, wherein the composition is a suspension formulation.

30. The composition of claim 29, wherein the composition comprises from about 1 to about 50 weight percent of the compound.

31. The composition of claim 30, further comprising sodium polycarboxylate.

32. The composition of claim 31, further comprising biocides.

33. The composition of claim 31, further comprising organosilicone antifoam emulsion.

34. The composition of claim 27, wherein the composition is a wettable powder.

35. The composition of claim 27, wherein the composition is an emulsifiable concentrate.

36. The composition of claim 35, further comprising tristyrylphenol ethoxylates.

37. The composition of claim 27, wherein the composition is an oil dispersible concentrate.

38. A pesticidal composition, comprising
a pesticide;
a compound of the formula 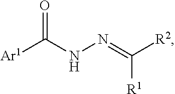
wherein Ar¹ is selected from aryl and heteroaryl;
R¹ is selected from hydrogen, C₃₋₆ cycloalkyl, C₁-6 alkyl, aralkyl, and C₁₋₃ haloalkyl;
R² is selected from alkyl, aryl and heteroaryl; provided that the compound does not have the formula 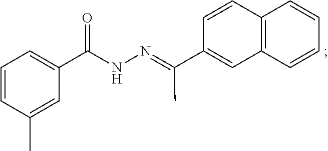 [SMILES: C/C(=N\NC(=O)c1cccc(C)c1)c1ccc2ccccc2c1]
and
a phytologically acceptable carrier.

39. The pesticidal composition of claim 38, wherein the pesticide comprises an acaricide, fungicide, herbicide, insecticide, molluscicide, nematocide, or a combination thereof.

40. A fungicidal composition, comprising
a fungicide;
a compound of the formula 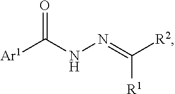
wherein Ar¹ is selected from aryl and heteroaryl;
R¹ is selected from hydrogen, C₃₋₆ cycloalkyl, C₁-6 alkyl, aralkyl, and C₁₋₃ haloalkyl;
R² is selected from alkyl, aryl and heteroaryl; provided that the compound does not have the formula 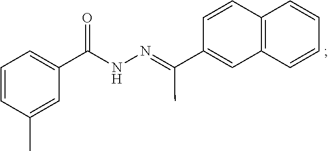 [SMILES: C/C(=N\NC(=O)c1cccc(C)c1)c1ccc2ccccc2c1]
and
a phytologically acceptable carrier.

41. The composition of claim 40, wherein the fungicide is selected from the group consisting of benzimidazoles, dicarboximides, phenylpyrroles, anilinopyrimidines, hydroxyanilides, carboxamides, phenyl amides, phosphonates, cinnamic acids, oxysterol binding protein inhibitors, triazole carboxamides, cymoxanil, carbamates, benzamides, demethylation inhibiting piperazines, demethylation inhibiting pyrimidines, demethylation inhibiting azoles, including imidazoles and triazoles, cyproconazole, difenoconazole, fenbuconazole, flutriafol, mefentrifluconazole, metconazole, ipconazole, prothioconazole, tebuconazole, tetraconazole, triadimefon, triadimenol, triticonazole, morpholines, cyflufenamid, metrafenone, pyriofenone, strobilurins, copper ammonium complex, copper hydroxide, copper oxide, copper oxychloride, copper sulfate, sulfur, lime sulfur, ethylenebisdithiocarbamates, aromatic hydrocarbons, phthalimides, guanidines, polyoxins, fluazinam, thiazolidines or a combination thereof.
